(19) United States
(12) Patent Application Publication
(10) Pub. No.: US 2011/0236393 A1
(43) Pub. Date: Sep. 29, 2011

(54) MONOCLONAL ANTIBODY PRODUCTION IN B CELLS AND USES THEROF

(75) Inventors: Kwan-ki Hwang, Chapel Hill, NC (US); Micah Luftig, Durham, NC (US)

(73) Assignee: Duke University

(21) Appl. No.: 13/127,627

(22) PCT Filed: Nov. 4, 2009
(86) PCT No.: PCT/US2009/063271

§ 371 Date: Jun. 9, 2011

Related U.S. Application Data
(60) Provisional application No. 61/198,217, filed on Nov. 4, 2008.

(51) Int. Cl. A61K 39/395; A61K 31/7088; C12N 15/86; C12N 5/0781; C12P 21/04; C07K 16/00; C07K 16/12; C07K 16/08; C07K 16/10; C07K 16/18; C12Q 1/02; C40B 50/06; A61K 31/7105; A61P 35/00; A61P 37/02

(52) U.S. Cl. 424/148.1; 514/44 A; 435/456; 435/375; 435/326; 435/71.1; 530/388.1; 530/388.4; 530/388.3; 530/388.35; 530/388.8; 424/141.1; 424/147.1; 424/149.1; 424/150.1; 424/155.1; 435/29; 506/26

(57) ABSTRACT

The presently disclosed subject matter provides methods of inhibiting a host innate response to activator-mediated proliferative signals in a primary B cell. In some embodiments a method is provided for immortalized primary B cells. In some embodiments a method is provided for increasing efficiency of EBV transformation of primary B cells. In some embodiments a method is provided for increasing proliferation of primary B cells in culture. In some embodiments a method is provided for producing a monoclonal antibody. In some embodiments a method is provided for identifying a novel broadly neutralizing antibody having a desired antigen specificity. Also provided are antibodies produced according the methods of the presently disclosed subject matter.

### CROSS-REFERENCE TO RELATED APPLICATIONS

This application claims benefit of U.S. Provisional Patent Application Ser. No. 61/198,217, entitled “Novel Approaches to Improving EBV Transformation Efficiency of HIV Antigen-Specific B Cells”, filed Nov. 4, 2008, which is herein incorporated by reference in its entirety.

### TECHNICAL FIELD

This presently disclosed subject matter generally relates to monoclonal antibodies, to methods for activating B cells, to methods for immortalizing B cells, and to methods for generating and identifying monoclonal antibodies with a desired antigen specificity. Also provided are therapeutic and diagnostic uses for the same. Further embodiments are described below.

### BACKGROUND

One approach to making human antibodies comprises the use of Epstein Barr Virus (EBV) to immortalize human (and primate) B cells producing specific antibodies. The EBV method has been described in several publications since 1977 (Rosen et al. 1977; Steinitz et al. 1977; Steinitz et al. 1980; Kozbor & Roder 1981; Lundgren et al. 1983; Rosen et al. 1983; Steinitz et al. 1984; Lanzavecchia 1985). However, the EBV-based method has several limitations, including the low efficiency of immortalization, the low cloning efficiency of EBV-immortalized B cells, the slow growth rate and, in some cases, low antibody production. U.S. Pat. No. 4,997,764 describes a method of improving the growth rate of the EBV immortalized cells comprising transfecting EBV infected B cells with activated c-myc DNA. While a number of these limitations have been addressed to some degree, the problem of low efficiency of immortalization remains. Consequently, the use of the EBV method has been significantly reduced or abandoned altogether.

Another reason why the EBV method has fallen out of favor is that alternative approaches for making human or human-like monoclonal antibodies became available through genetic engineering. These include the humanization of murine antibodies, the isolation of antibodies from libraries of different complexity and the production of hybridomas using the classical method in mice transgenic for human Ig loci (the“xeno-mouse”). However, these methods also have limitations. Humanization of murine monoclonal antibodies is a laborious and incomplete procedure. Random antibody libraries represent an unbiased repertoire and can therefore be used to select antibody specificities against highly conserved antigens, but can lead to antibodies of low affinity. Libraries selected from antigen primed B cells are enriched for a particular specificity, but do not preserve the original VH-VL pairing and generally lead to antibodies that have lower affinity for the antigen than those present in the original antibody repertoire. The impact of this technology has been limited. In contrast the xeno-mouse can be efficiently immunized against an antigen of choice (especially if this is a human antigen), but this system shares with the classical murine hybridoma technology the limitation that the antibodies are selected in a species other than human. Therefore these methods often are not suitable to produce antibodies with the characteristics of those produced in the course of a physiological human immune response. This applies, for example, to the antibody response to human pathogens including HIV, the four Plasmodium species that cause malaria in humans (P. falciparum, P. vivax, P. malariae and P. ovale), human hepatitis B and C viruses, Measles virus, Ebola virus etc. (see Fields et al. 1996). By way of additional examples, it also applies to antibody responses to environmental allergens generated in allergic patients, to tumor antigens generated in tumor bearing patients and to self antigens in patients with autoimmune diseases.

Accordingly, there remains an unmet need for an efficient method of producing human monoclonal antibodies that have been selected in the course of the natural immune response.

### SUMMARY

In some embodiments, the presently disclosed subject matter provides a method of inhibiting a host innate response to activator-mediated proliferative signals in a primary B cell, the method comprising administering to a cell an inhibitor of a host innate response to activator-mediated proliferative signals. In some embodiments, the host innate response to activator-mediated proliferative signals comprises a DNA damage response (DDR) pathway. In some embodiments, the inhibitor of a host innate response to activator-mediated proliferative signals comprises a small molecule, a nucleic acid molecule capable of mediating RNA interference, or combinations thereof. In some embodiments, the small molecule, nucleic acid molecule capable of mediating RNA interference, or combinations thereof, inhibits a component of the DDR pathway. In some embodiments, the component of the DDR pathway comprises Chk1, Chk2, ATM, ATR, DNA-PK, PARP family members, Tip60 or combinations thereof. In some embodiments, the primary B cell is a human primary B cell.

In some embodiments, the presently disclosed subject matter provides a method for producing immortalized primary B cells, the method comprising transforming primary B cells using Epstein Barr virus (EBV) in the presence of an inhibitor of a host innate response to EBV-mediated proliferative signals. In some embodiments, the host innate response to activator-mediated proliferative signals comprises a DNA damage response (DDR) pathway. In some embodiments, the inhibitor of a host innate response to activator-mediated proliferative signals comprises a small molecule, a nucleic acid molecule capable of mediating RNA interference, or combinations thereof. In some embodiments, the small molecule, nucleic acid molecule capable of mediating RNA interference, or combinations thereof, inhibits a component of the DDR pathway. In some embodiments, the component of the DDR pathway comprises Chk1, Chk2, ATM, ATR, DNA-PK, PARP family members, Tip60 or combinations thereof. In some embodiments, the primary B cell is a human primary B cell. In some embodiments, the primary B cell is obtained from a subject infected with or possessing an antigen of interest.

In some embodiments, the presently disclosed subject matter provides a method for increasing efficiency of EBV transformation of primary B cells, the method comprising: (i) providing a primary B cell; and (ii) transforming the primary B cell using EBV in the presence of an inhibitor of a host innate response to EBV-mediated proliferative signals, wherein the efficiency of the EBV transformation of the primary B cell is increased compared to EBV transformation without the use of an inhibitor of a host innate response to EBV-mediated proliferative signals. In some embodiments, a host innate response to activator-mediated proliferative signals comprises the DNA damage response (DDR) pathway. In some embodiments, the inhibitor of a host innate response to activator-mediated proliferative signals comprises a small molecule, a nucleic acid molecule capable of mediating RNA interference, or combinations thereof. In some embodiments, the small molecule, a nucleic acid molecule capable of mediating RNA interference, or combinations thereof, inhibits a component of the DDR pathway. In some embodiments, the component of the DDR pathway comprises Chk1, Chk2, ATM, ATR, DNA-PK, PARP family members, Tip60 or combinations thereof. In some embodiments, the primary B cell is a human primary B cell. In some embodiments, the primary B cell is obtained from a subject infected with or possessing an antigen of interest.

In some embodiments, the presently disclosed subject matter provides a method for increasing proliferation of primary B cells in culture, the method comprising providing to a primary B cell in culture an activator of B cell proliferation in the presence of an inhibitor of a host innate response to activator-mediated proliferative signals. In some embodiments, the host innate response to activator-mediated proliferative signals comprises a DNA damage response (DDR) pathway. In some embodiments, the inhibitor of a host innate response to activator-mediated proliferative signals comprises a small molecule, a nucleic acid molecule capable of mediating RNA interference, or combinations thereof. In some embodiments, the small molecule, nucleic acid molecule capable of mediating RNA interference, or combinations thereof, inhibits a component of the DDR pathway. In some embodiments, the component of the DDR pathway comprises Chk1, Chk2, ATM, ATR, DNA-PK, PARP family members, Tip60 or combinations thereof. In some embodiments, the activator is a polyclonal activator. In some embodiments, the polyclonal activator comprises an agonist of a Toll Like Receptor (TLR) which is expressed on B cells. In some embodiments, the TLR is selected from the group comprising TLR-7, TLR-9, TLR-10 and combinations thereof. In some embodiments, the polyclonal activator is selected from the group comprising CpG oligodeoxynucleotide; R-848; CD40L; BAFF; cells that express CD40L or BAFF; and monoclonal antibodies, small molecules, or nucleic acids that mimic one or more effects of the foregoing activators. In some embodiments, the activator is a B cell mitogen or combinations of B cell mitogens. In some embodiments, the B cell mitogen or combination of B cell mitogens is selected from the group comprising mitogenic cytokine, IL-2, IL-4, IL-5, IL-10, IL-15, CD40L, a CD40 agonist, and anti-Ig. In some embodiments, the activator is a CpG oligodeoxynucleotide and the inhibitor is a Chk2 inhibitor. In some embodiments, the activator is EBV. In some embodiments, EBV transforms the primary B cell. In some embodiments, the primary B cell is a human primary B cell.

In some embodiments, the presently disclosed subject matter provides a method for producing a clone of an immortalized primary B cell capable of producing a monoclonal antibody with a desired antigen specificity, the method comprising: (i) transforming a population of cells comprising primary B cells with EBV in the presence of an inhibitor of a host innate response to EBV-mediated proliferative signals; (ii) screening the supernatant from the population of cells for antigen specificity; and (iii) isolating from the population of cells an immortalized B cell clone capable of producing a monoclonal antibody having the desired antigen specificity. In some embodiments, the host innate response to EBV-mediated proliferative signals comprises a DNA damage response (DDR) pathway. In some embodiments, the inhibitor of a host innate response to EBV-mediated proliferative signals comprises a small molecule, a nucleic acid molecule capable of mediating RNA interference, or combinations thereof. In some embodiments, the small molecule, nucleic acid molecule capable of mediating RNA interference, or combinations thereof, inhibits a component of the DDR pathway. In some embodiments, the component of the DDR pathway comprises Chk1, Chk2, ATM, ATR, DNA-PK, PARP family members, Tip60 or combinations thereof. In some embodiments, the population of cells comprising primary B cells is derived from a human. In some embodiments, the desired antigen specificity of the antibody is directed to a pathogen, allergen, tumor antigen, autoantigen, alloantigen, microbial pathogen, chemical or toxin. In some embodiments, the desired antigen specificity of the antibody is against a pathogen selected from the group comprising HIV, P. falciparium, P. vivax, P. malariae, P. ovale, hepatitis A, hepatitis B, hepatitis C, Measles virus, Ebola virus, Poxviruses, Influenza virus, H1N1 virus, Avian influenza virus, herpes simplex virus type 1 or type 2; SARS coronavirus, mumps virus, rubella virus, rabies virus, papillomavirus, vaccinia virus, varicella-zoster virus, variola virus, polio virus, rhinoviruses, respiratory syncytial virus, a human endogenous retroviruses, Dengue virus, Corynebacterium diphtheriae, Clostridium tetani, Clostridium botulinum, Bordetella pertussis, Haemophilus influenzae, Neisseria meningitidis, serogroup A, B, C, W135 and/or Y; Streptococcus pneumoniae, Streptococcus agalactiae, Streptococcus pyogenes, Staplhylococcus aureus, Bacillus anthracis, Moraxella catarrhalis, Chlaymdia trachomatis, Chlamydia pneumoniae, Yersinia pestis, Francisella tularensis, Salmonella species, Vibrio cholerae, toxic E. coli, and other viral or microbial pathogen. In some embodiments, the presently disclosed subject matter provides a clone of an immortalized primary B cell produced according to the methods of the presently disclosed subject matter.

In some embodiments, the presently disclosed subject matter provides a method for producing a monoclonal antibody, comprising the steps of: (i) providing to a primary B cell in culture an activator of B cell proliferation in the presence of an inhibitor of a host innate response to activator-mediated proliferative signals; (ii) screening the supernatant from the culture for antigen specificity; (iii) isolating from the culture a B cell capable of producing a monoclonal antibody having the desired antigen specificity; and (iv) expressing the monoclonal antibody, comprising (a) culturing the B cell under conditions where the monoclonal antibody is expressed; or (b) obtaining a nucleic acid encoding the antibody of interest from the isolated B cell; (c) employing the nucleic acid to prepare an expression host that can express the antibody of interest; and (d) culturing the expression host under conditions where the antibody of interest is expressed. In some embodiments, the host innate response to activator-mediated proliferative signals comprises a DNA damage response (DDR) pathway. In some embodiments, the inhibitor of a host innate response to actvator-mediated proliferative signals comprises a small molecule, a nucleic acid molecule capable of mediating RNA interference, or combinations thereof. In some embodiments, the small molecule, nucleic acid molecule capable of mediating RNA interference, or combinations thereof, inhibits a component of the DDR pathway. In some embodiments, the component of the DDR pathway comprises Chk1, Chk2, ATM, ATR, DNA-PK, PARP family members, Tip60 or combinations thereof. In some embodiments, the population of cells comprising primary B cells is derived from a human. In some embodiments, the activator is a polyclonal activator. In some embodiments, the polyclonal activator comprises an agonist of a Toll Like Receptor (TLR) that is expressed on B cells. In some embodiments, the TLR is selected from the group comprising TLR-7, TLR-9, TLR-10 and combinations thereof. In some embodiments, the polyclonal activator is selected from the group comprising CpG oligodeoxynucleotide; R-848; CD40L; BAFF; cells that express CD40L or BAFF; and monoclonal antibodies, small molecules, or nucleic acids that mimic the effects of these activators. In some embodiments, the activator is a mitogenic cytokine. In some embodiments, the B cell mitogen or combination of B cell mitogens is selected from the group comprising mitogenic cytokine, IL-2, IL-4, IL-5, IL-10, IL-15, CD40L, a CD40 agonist, and anti-Ig. In some embodiments, the activator is EBV. In some embodiments, EBV transforms the primary B cell. In some embodiments, the desired antigen specificity of the antibody is directed to a pathogen, allergen, tumor antigen, autoantigen, alloantigen, microbial pathogen, chemical or toxin. In some embodiments, the desired antigen specificity of the antibody is against a pathogen selected from the group comprising HIV, P. falciparium, P. vivax, P. malariae, P. ovale, hepatitis A, hepatitis B, hepatitis C, Measles virus, Ebola virus, Poxviruses, Influenza virus, H1N1 virus, Avian influenza virus, herpes simplex virus type 1 or type 2; SARS coronavirus, mumps virus, rubella virus, rabies virus, papillomavirus, vaccinia virus, varicella-zoster virus, variola virus, polio virus, rhinoviruses, respiratory syncytial virus, a human endogenous retroviruses, Dengue virus, Corynebacterium diphtheriae, Clostridium tetani, Clostridium botulinum, Bordetella pertussis, Haemophilus influenzae, Neisseria meningitidis, serogroup A, B, C, W135 and/or Y; Streptococcus pneumoniae, Streptococcus agalactiae, Streptococcus pyogenes, Staplhylococcus aureus, Bacillus anthracis, Moraxella catarrhalis, Chlaymdia trachomatis, Chlamydia pneumoniae, Yersinia pestis, Francisella tularensis, Salmonella species, Vibrio cholerae, toxic E. coli, and other viral or microbial pathogen. In some embodiments, the method further comprises (v) purifying the monoclonal antibody. In some embodiments, the expression host is a prokaryotic or eukaryotic cell. In some embodiments, the presently disclosed subject matter provides a monoclonal antibody produced according to the methods of the presently disclosed subject matter. In some embodiments, the presently disclosed subject matter provides a pharmaceutical composition comprising a pharmaceutically acceptable carrier and a monoclonal antibody or functional fragment thereof produced according the methods of the presently disclosed subject matter. In some embodiments, the presently disclosed subject matter provides a method of treating a subject, comprising administering to the subject a pharmaceutical composition of the presently disclosed subject matter. In some embodiments, the presently disclosed subject matter provides a method of diagnosing a subject, comprising employing a monoclonal antibody produced according to the methods of the presently disclosed subject matter.

In some embodiments, the presently disclosed subject matter provides a method for identifying a novel broadly neutralizing antibody having a desired antigen specificity, comprising the steps of: (i) providing a subject infected with or possessing the antigen for which the antibody specificity is desired; (ii) culturing primary B cells obtained from the subject; (iii) providing to the cultured primary B cells an activator of B cell proliferation in the presence of an inhibitor of a host innate response to activator-mediated proliferative signals; and (iv) screening the supernatant from the culture of proliferating primary B cells for a novel broadly neutralizing antibody having the desired antigen specificity. In some embodiments, the host innate response to activator-mediated proliferative signals comprises the DNA damage response (DDR) pathway. In some embodiments, the inhibitor of a host innate response to activator-mediated proliferative signals comprises a small molecule, a nucleic acid molecule capable of mediating RNA interference, or combinations thereof. In some embodiments, the small molecule, nucleic acid molecule capable of mediating RNA interference, or combinations thereof, inhibits a component of the DDR pathway. In some embodiments, the component of the DDR pathway comprises Chk1, Chk2, ATM, ATR, DNA-PK, PARP family members, Tip60 or combinations thereof. In some embodiments, the population of cells comprising primary B cells is derived from a human. In some embodiments, the activator is a polyclonal activator. In some embodiments, the polyclonal activator comprises an agonist of a Toll Like Receptor (TLR) which is expressed on B cells. In some embodiments, the TLR is selected from the group comprising TLR-7, TLR-9, TLR-10 and combinations thereof. In some embodiments, the polyclonal activator is selected from the group comprising CpG oligodeoxynucleotide; R-848 and other compounds that stimulate TLRs; CD40L; BAFF; cells that express CD40L or BAFF; and monoclonal antibodies, small molecules, or nucleic acids that mimic the effects of these activators. In some embodiments, the activator is a mitogenic cytokine. In some embodiments, the B cell mitogen or combination of B cell mitogens is selected from the group comprising mitogenic cytokine, IL-2, IL-4, IL-5, IL-10, IL-15, CD40L, a CD40 agonist, and anti-Ig. In some embodiments, the activator is EBV. In some embodiments, EBV transforms the primary B cells. In some embodiments, the desired antigen specificity of the antibody is directed to any human pathogen, allergen, tumor antigen, autoantigen, alloantigen, microbial pathogen, chemical or toxin. In some embodiments, the desired antigen specificity of the antibody is selected from the group comprising HIV, P. falciparium, P. vivax, P. malariae, P. ovale, hepatitis A, hepatitis B, hepatitis C, Measles virus, Ebola virus, Poxviruses, Influenza virus, H1N1 virus, Avian influenza virus, herpes simplex virus type 1 or type 2; SARS coronavirus, mumps virus, rubella virus, rabies virus, papillomavirus, vaccinia virus, varicella-zoster virus, variola virus, polio virus, rhinoviruses, respiratory syncytial virus, a human endogenous retroviruses, Dengue virus, Corynebacterium diphtheriae, Clostridium tetani, Clostridium botulinum, Bordetella pertussis, Haemophilus influenzae, Neisseria meningitidis, serogroup A, B, C, W135 and/or Y; Streptococcus pneumoniae, Streptococcus agalactiae, Streptococcus pyogenes, Staplhylococcus aureus, Bacillus anthracis, Moraxella catarrhalis, Chlaymdia trachomatis, Chlamydia pneumoniae, Yersinia pestis, Francisella tularensis, Salmonella species, Vibrio cholerae, toxic E. coli, and other viral or microbial pathogen. In some embodiments, the method further comprises (v) purifying the novel broadly neutralizing antibody having a desired antigen specificity. In some embodiments, the presently disclosed subject matter provides a novel broadly neutralizing antibody having a desired antigen specificity produced according to the methods of the presently disclosed subject matter. In some embodiments, the presently disclosed subject matter provides a pharmaceutical composition comprising a pharmaceutically acceptable carrier and a novel broadly neutralizing antibody or functional fragment thereof produced according the presently disclosed subject matter. In some embodiments, the presently disclosed subject matter provides a method of treating a subject, comprising administering to the subject a pharmaceutical composition of the presently disclosed subject matter. In some embodiments, the presently disclosed subject matter provides a method of diagnosing a subject, comprising employing the novel broadly neutralizing antibody produced according to the presently disclosed subject matter. In some embodiments, the method further comprises the steps of: (v) isolating from the culture a B cell clone capable of producing a novel broadly neutralizing antibody having a desired antigen specificity; and (vi) culturing the B cell clone under conditions where the novel broadly neutralizing antibody having a desired antigen specificity is expressed. In some embodiments, the method further comprises the steps of: (v) isolating from the culture a B cell clone capable of producing a novel broadly neutralizing antibody having a desired antigen specificity; (vi) obtaining and/or sequencing a nucleic acid for the novel broadly neutralizing antibody from the selected B cell clone; (vii) employing the nucleic acid to prepare an expression host that can express the antibody of interest; and (viii) culturing the expression host under conditions where the novel broadly neutralizing antibody having a desired antigen specificity is expressed. In some embodiments, the expression host is a prokaryotic or eukaryotic cell.

In some embodiments, the presently disclosed subject matter provides a method for generating a profile of the humoral response of a subject, comprising the steps of: (i) culturing primary B cells obtained from a subject; (ii) providing to the cultured primary B cells an activator of B cell proliferation in the presence of an inhibitor of a host innate response to activator-mediated proliferative signals; (iii) screening the supernatant from the culture of proliferating primary B cells for one or more antibodies; and (iv) characterizing the one or more antibodies, wherein a profile of the humoral response of the subject is generated. In some embodiments, characterizing the one or more antibodies comprises collecting data regarding the specificity, affinity, stability, isotypte, or gene segment sequence preference of the one or more antibodies.

In some embodiments, the nucleic acid molecule capable of mediating RNA interference is selected from the group comprising small interfering RNA, short interfering RNA, microRNA, short hairpin RNA, and combinations thereof.

It is an object of the presently disclosed subject matter to provide a method of producing monoclonal antibodies from B cells. This and others objects are achieved in whole or in part by the presently disclosed subject matter.

An object of the presently disclosed subject matter having been stated above, other objects and advantages of the presently disclosed subject matter will become apparent to those skilled in the art after a study of the following description and Examples.

### BRIEF DESCRIPTION OF THE DRAWINGS

FIG. 1 is a schematic drawing illustrating the effects of EBV infection of primary B cells on the expression of a subset of viral genes and the host pathways they usurp to drive B cell growth and ensure survival.

FIG. 2 is a schematic drawing of a model for EBV-induced DNA damage response (DDR) limiting long-term immortalization. The model presents a molecular and functional view of the EBV-induced oncogenic stress response. As disclosed herein, EBV induced hyper S phase induction early after B cell infection is responsible for DNA damage that ultimately suppresses proliferation. This proliferative block is in turn responsible for the less than 10% efficiency of transformation. Cells that have attenuated this hyper S-phase induction are then able to grow out as LCLs.

FIGS. 3A and 3B are graphical plots showing the inhibition of ATM and Chk2 kinases increased EBV transformation efficiency. FIG. 3A shows the quantification of EBV-induced outgrowth following PBMC infection in the presence of 0.1% DMSO (diamond), 2 μM ATMi (square), or 5 μM ATMi (triangle). The percentages of wells positive for LCLs at six weeks post infection were plotted relative to the amount of B95-8 virus used per well. The efficiency of transformation was determined by the amount of virus necessary to induce 63% positive wells as indicated by a Poisson's distribution. This value was set to 1 for the DMSO treated infections. The difference between DMSO and 5 μM ATMi treated infections was ˜30-fold increased efficiency in the presence of 5 μM ATMi. In FIG. 3B similar experiments were performed using DMSO (diamond), 2 μM Chk2i II (square), or 5 μM Chk2i II (triangle). Results are displayed as in FIG. 3A.

FIGS. 4A, 4B and 4C show inhibition of ATM or Chk2 increased the proliferation of B cells following EBV infection. FIG. 4A is a dot plot of EBV-mediated proliferation following infection of CFSE-stained PBMC, detected by FACS using CD19-PE staining of B cells and CFSE to monitor cell divisions, at 2 and 6 days post infection. At day 2 post infection there was little proliferation, while by day 6 there was clear B cell proliferation induced by the virus. FIG. 4B is a histogram illustrating the effect of CFSE-stained PBMC infected with EBV in the presence of increasing amounts of ATMi or Chk2i (0.1% DMSO, 1 μM, 2 μM, 5 μM, and 10 μM). The percentage of CD19+/CFSElo cells of total PBMCs at 14 days post infection are plotted. The data shown are the average values from two different normal donors with error bars reflecting the standard error. These data are representative of more than five independent experiments. FIG. 4C includes dot plots of the proliferation of CFSE-stained PBMC treated with DMSO, 5 μM ATMi, 5 μM Chk21, or infected with EBV. Six days post treatment/infection cells were stained with CD19-PE antibody and proliferation of B cells was assessed. DDR inhibition did not induce B cells proliferation. The difference in relative B cell proliferation at day six between FIGS. 4A and 4C was due to normal donor variation.

FIGS. 5A, 5B and 5C show EBV induced rapid proliferation early after infection followed by attenuated proliferative cycles. FIG. 5A is a dot plot of PBMC infected with EBV and analyzed by CD19-APC staining and CFSE proliferation dye. The infected PBMCs were first gated on live cells (not shown) followed by gating on CD19+ cells as shown (CD19 expression decreases subtly over time). These cells were then analyzed for CFSE levels in the histogram in FIG. 5B. The histogram of FIG. 5B shows gates calculated to represent population doublings, or halving of CFSE fluorescence intensity. The gates are numbered above with the number of population doublings. Note that the x-axis is shown in logarithmic scale. FIG. 5C includes dot plots of EBV-induced proliferation observed at days 4, 5, 6, 8, 9 and 11. EBV-induced proliferation was observed starting at day 5. However, these populations already contained cells that have proliferated 3 and 4 times. This indicates that infected cells proliferated at a very high rate between day 4 and day 5. Subsequent proliferation was slower, displaying an attenuated cell cycle similar to that found in indefinitely proliferating LCLs.

FIGS. 6A and 6B are graphical depictions of the proliferation rate of EBV-infected cells early after infection. PBMC infected with EBV were analyzed by CD19+ staining and CFSE proliferation dye. Population doublings (0-7) were determined for CD19+ cells for 78-120 hours post infection. In the histogram of FIG. 6A, cell progeny number (N) was determined as the number of cells at given division (PDi) normalized to division number (N=PDi/i) and fitted to a Gaussian distribution for each time point. Since one population doubling has cells at different cell cycle progression (i.e. divided from 0% to 99%), the division number (i) was averaged as i=i+0.5. The number of cells normalized and plotted over division number, including putative “negative” divisions, generates the PRECURSOR COHORT (representative distributions are shown for 3 time points). In the plots of FIG. 6B, mean division numbers were determined by the mean of Gaussian curves from FIG. 6A and plotted over time post infection. The proliferation rate equals 1 divided by the slope of the curve, whereas the first division time equals the coordinate where the function crosses the x-axis (˜73 hours post infection). In the top panel of FIG. 6B, slope of EBV proliferation changes from 78 to 126 hours displaying a bi-phasic proliferation pattern. In the bottom panels of FIG. 6B (left to right), during the period from 78-102 h post infection cells divided approximately every 12 hours. After ˜100 hours post infection cells divided approximately every 31 hours. These data are representative of 2 experiments performed with 3 normal donors.

FIG. 7 is a bar graph of the proliferative potency of sorted EBV-infected B cells. PBMC infected with EBV were analyzed by CD19 staining and CFSE proliferation dye. Percent of proliferating cells is plotted over sorted population doubling number. Percent of cells divided for more than 1, 2 or 3 times was determined for each sorted population (PD0-3) kept in culture for 24 hours. During this period cells that divided more than 1 time are plotted in gray, more than 2 times in dark gray and more than 3 times in light gray.

FIGS. 8A, 8B and 8C show EBV induced γ-H2AX foci in early, but not late proliferating cells. FIG. 8A comprises immunofluorescent images of the indirect immunofluorescence of EBNA-LP and γ-H2AX of sorted PD0, PD1, and PD6 B cells. DNA was stained with DAPI. Immunofluorescence of uninfected B cells and established LCLs are provided for comparison. FIG. 8B is a bar graph of the percentage of EBNA-LP positive cells from immunofluorescence. Individual cells were defined as positive if their mean EBNA-LP levels were greater than two times the average level of all uninfected cells. FIG. 8C is a bar graph of the percentage of EBNA-LP positive cells from FIG. 8B that were positive for γ-H2AX. Individual cells were defined as being γ-H2AX positive if their mean γ-H2AX levels were greater than 1.5 times the average of all uninfected cells. The dotted line indicates the background level of γ-H2AX positively in uninfected cells.

FIG. 9 is scatter plot of the effects of inhibition of the DDR, in particular Chk2, on the efficiency of EBV transformation of memory B cells.

FIG. 10 is scatter plot of the effects of inhibition of the DDR, in particular Chk2, on the frequency of antibody secreting cells and their Ig production.

FIG. 11 is bar graph showing that Chk2 increased the total B cell number following CpG treatment or EBV infection. PBMC were stimulated with CpG 2006 (2.5 μg/mL) or infected with EBV (B95-8, MOI 5). CD19+ B cells were assayed 7 days after treatment of infection using flow cytometry. The total number of viable CD19+ B cells is plotted.

FIG. 12 is a histogram showing that Chk2 inhibition increased B cell number following CpG or CpG system stimulation. Cells were assayed at different time points after stimulation. Circles are CpG treated samples and squares are CpG system treated samples. Open symbols are DMSO-treated and closed symbols are Chk21-treated.

FIG. 13 is a scatter plot of the frequency of IgG positive cell clones of EBV transformed memory B cells from chronic HIV-1-infected patients in the presence or absence of Chk2i II. An increase in the frequency of IgG positive cell clones in the presence of Chk2i II was observed.

FIG. 14 is a bar graph showing that SMB2-2 IgG binds to gp120 of HIV gp140 Env protein. Purified IgG at the indicated concentrations was tested for its reactivity to HIV-1 Env proteins in ELISA. The data are expressed in OD.

FIG. 15 is a bar graph showing that SMB2-2 IgG captures HIV-1 virions, SF162 and BG1168. Purified IgG was coated on a microtiter plate. The amount of virions captured by the test IgG was measured by a standard p24 ELISA.

FIG. 16 is a plot showing neutralized infection of HIV-1 strains SF162 and MN in vitro. SMB2-2 IgG neutralized HIV-1 strains, MN and SF162. Purified IgG at the indicated concentrations was tested for its potency to inhibit HIV-1 (MN and SF162) infectivity in a TZM-bl-based neutralization assay. SVA, negative control virus.

### BRIEF SUMMARY OF THE SEQUENCE LISTING

SEQ ID NO. 1 is a polynucleotide sequence of CpG 2006.

### DETAILED DESCRIPTION

### I. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “cell” refers not only to the particular subject cell (e.g., a living biological cell), but also to the progeny or potential progeny of such a cell. Because certain modifications can occur in succeeding generations due to either mutation or environmental influences, such progeny might not, in fact, be identical to the parent cell, but are still included within the scope of the term as used herein.

The term “ligand” as used herein refers to a molecule or other chemical entity having a capacity for binding to a target. A ligand can comprise a peptide, an oligomer, a nucleic acid (e.g., an aptamer), a small molecule (e.g., a chemical compound), an antibody or fragment thereof, a nucleic acid-protein fusion, and/or any other affinity agent.

The term “small molecule” as used herein refers to a compound, for example an organic compound, with a molecular weight in some embodiments of less than about 1,000 daltons, in some embodiments less than about 750 daltons, in some embodiments less than about 600 daltons, and in some embodiments less than about 500 daltons. A small molecule also has a computed log octanol-water partition coefficient in some embodiments in the range of about −4 to about +14, and in some embodiments in the range of about −2 to about +7.5.

The term “target tissue” as used herein refers to an intended site for accumulation of a ligand following administration to a subject. For example, the methods disclosed herein can employ a target tissue comprising a tumor or cancerous tissues.

As used herein, the terms “B cell”, “B memory cell”, “B lymphocyte”, “B memory lymphocyte”, “memory cells”, “memory B cell”, and variants thereof are used interchangeably and refer to B cells of the humoral immune response. As would be appreciated by one of ordinary skill in the art, B cells are lymphocytes that play a role in the humoral immune response (as opposed to the cell-mediated immune response, which is governed by T cells). At least one function of B cells is to make antibodies against antigens, perform the role of Antigen Presenting Cells (APCs) and eventually develop into memory B cells after activation by antigen interaction. B cells are a component of the adaptive immune system.

As used herein, the phrase “primary B cell” can refer in some embodiments to a B cell taken directly from a living organism (e.g. a human). In some embodiments, a primary B cell can be cultured in a primary cell culture. A primary B cell can be derived, obtained or collected from a subject in any manner known to those of skill in the art. In some embodiments, a primary B cell is obtained from a subject infected with or possessing an antigen of interest.

As used herein, the term “proliferation” refers to the replication, growth, and/or survival of a cell. In some embodiments proliferation refers to replication, growth, and/or survival of B cells in culture. In some embodiments, proliferation of cells can increase in response to a treatment or method as disclosed herein. An increase in proliferation refers to an increase in replication, growth, and/or survival or cells as compared to a basal rate or starting point, or in the absence of a treatment or method.

The term “control tissue” as used herein refers to a site suspected to substantially lack binding and/or accumulation of an administered ligand. For example, in accordance with the methods of the presently disclosed subject matter, a non-cancerous tissue can be a control tissue.

The terms “target” or “target molecule” as used herein each refer to any substance that is selectively bound by a ligand or antibody. Thus, the term “target molecule” encompasses macromolecules including but not limited to proteins (e.g., receptors), nucleic acids, carbohydrates, lipids, and complexes thereof.

The terms “targeting” or “homing”, as used herein to describe the in vivo activity of a ligand or antibody following administration to a subject, each refer to the preferential movement and/or accumulation of a ligand in a target tissue as compared with a control tissue.

The term “binding” refers to an affinity between two molecules, for example, a ligand and a target molecule. As used herein, “binding” means a preferential binding of one molecule for another in a mixture of molecules. In some embodiments, the binding of a ligand to a target molecule can be considered specific or selective if the binding affinity is in some embodiments about 1×10⁴ M⁻¹ to about 1×10⁶ M⁻¹ or greater.

The phrase “specifically (or selectively) binds”, when referring to the binding capacity of a ligand, refers to a binding reaction which is determinative of the presence of the target in a heterogeneous population of other biological materials. The phrase “specifically (or selectively) binds” also refers to selectively targeting, as defined hereinabove.

The phases “substantially lack binding” or “substantially no binding”, as used herein to describe binding of a ligand in a control tissue, refers to a level of binding that encompasses non-specific or background binding, but does not include specific binding.

The terms “humanized” or “humanized antibody”, as used herein, refers to an antibody derived from a non-human antibody, for example but not limited to murine, that retains or substantially retains the antigen-binding properties of the parent antibody but which is less immunogenic in humans than a non-humanized antibody.

The term “tumor” as used herein refers to both primary and metastasized solid tumors and carcinomas of any tissue in a subject, including but not limited to breast; colon; rectum; lung; oropharynx; hypopharynx; esophagus; stomach; pancreas; liver; gallbladder; bile ducts; small intestine; urinary tract including kidney, bladder and urothelium; female genital tract including cervix, uterus, ovaries (e.g., choriocarcinoma and gestational trophoblastic disease); male genital tract including prostate, seminal vesicles, testes and germ cell tumors; endocrine glands including thyroid, adrenal, and pituitary; skin (e.g., hemangiomas and melanomas), bone or soft tissues; blood vessels (e.g., Kaposi's sarcoma); brain, nerves, eyes, and meninges (e.g., astrocytomas, gliomas, glioblastomas, retinoblastomas, neuromas, neuroblastomas, Schwannomas and meningiomas). The term “tumor” also encompasses solid tumors arising from hematopoietic malignancies such as leukemias, including chloromas, plasmacytomas, plaques and tumors of mycosis fungoides and cutaneous T-cell lymphoma/leukemia, and lymphomas including both Hodgkin's and non-Hodgkin's lymphomas.

The term “subject” as used herein refers to any invertebrate or vertebrate species. The methods and compositions disclosed herein are particularly useful in the treatment and diagnosis of warm-blooded vertebrates. Thus, the presently disclosed subject matter concerns mammals and birds. More particularly provided is the treatment and/or diagnosis of mammals such as humans, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, provided is the treatment of livestock, including, but not limited to, domesticated swine (pigs and hogs), ruminants, horses, poultry, and the like.

In some embodiments the presently disclosed subject matter has been described in relation to human antibodies prepared from human B cells. It will be appreciated that the presently disclosed subject matter is not restricted to the use of human antibodies, cells, or subjects, and can be used with any organism of interest, e.g. to provide antibodies for therapeutic or diagnostic veterinary use. As would be appreciated by one of ordinary skill in the art, organisms with B cells that can be transformed or activated by the methods of the presently disclosed subject matter are within the scope of the instant disclosure.

### II Activating B Cells

The presently disclosed subject matter provides in some embodiments the activation of B cells (e.g. primary B cells) coupled with the inhibition of a host innate response to activator-mediated proliferative signals to enhance B cell proliferation. In enhancing B cell proliferation by activating B cells and inhibiting a host innate response to activator-mediated proliferative signals the presently disclosed subject matter provides in some embodiments for the rapid isolation of large numbers of monoclonal antibodies (e.g. human monoclonal antibodies) from the memory repertoire of a subject with no need for specific immunization or boosting. In some embodiments B cell activation comprises Epstein Barr virus (EBV) transformation. In some embodiments B cell activation comprises exposing B cells to one or more polyclonal activators or mitogens, e.g. mitogenic cytokines, or combinations thereof. In some embodiments inhibition of a host innate response to activator-mediated proliferative signals comprises inhibiting the DNA damage response (DDR) pathway.

In some embodiments, the presently disclosed subject matter couples the interference with host innate response to activator-mediated proliferative signals, also known as innate tumor suppressor pathways, in order to increase the efficiency of B cell proliferation and/or immortalization procedures. In particular, inhibition of the DNA damage response (DDR) pathway in conjunction with B cell activation provides for the optimization of B cell proliferation. When coupled with EBV transformation, inhibition of the DDR pathway in B cells provides for substantially increased B cell proliferation and in some embodiments substantially improved immortalization. As described further below, inhibition of components of the DDR, e.g. the kinases ATM, ATR, Chk1, Chk2, or DNA-PK, PARP family poly ADP-ribose polymerases, and/or Tip60 (acetyltransferase), results in marked increases in transformation efficiency relative to EBV infection in the absence of inhibitors.

This increased B cell proliferation or efficiency of immortalization provides for the interrogation of the human memory B cell pool from subjects producing antibodies of potential commercial, therapeutic and diagnostic interest. This includes those subjects infected with pathogens of interest to identify neutralizing antibodies to prevent infection or inform vaccine design or individuals with diseases that can elicit self-antibodies of therapeutic potential. For the latter point, self-antibodies that can interfere or augment inflammatory responses can be useful for the treatment of such diseases (e.g., rheumatoid arthritis, lupus, etc). Similarly, tumor antigens can be targets of such antibodies in cancer patients. By way of example and not limitation, tumor antigens can comprise EGFR, MUC1, and CEA. Additionally, the presently disclosed subject matter provides for the ability to sample the memory B cell repertoire (e.g. human memory B cell repertoire) for antibodies that can have therapeutic potential or information directing vaccine design. The methods of the presently disclosed subject matter involve separating B cells from such subjects followed by B cell activation in the presence of an inhibitor of host innate response to activator-mediated proliferation signals, such as an inhibitor of the DDR. Such methods include robust transformation of small numbers of B cells, which can subsequently be interrogated for properties of interest (e.g., antigen specificity and affinity).

The memory B cells to be activated can come from various sources (e.g. from whole blood, from peripheral blood mononuclear cells (PBMCs), from blood culture, from bone marrow, from organs, etc.), and suitable methods for obtaining B cells (e.g. human B cells) are known in the art. In some embodiments samples can comprise B cells as well as cells that are not memory B cells, e.g. other blood cells. A specific memory B cell subpopulation (e.g. human memory B lymphocyte subpopulation) exhibiting a desired antigen specificity can be selected before the transformation step by using methods known in the art. By way of example and not limitation, a human memory B lymphocyte subpopulation employed in the presently disclosed subject matter can have specificity for the HIV virus, e.g. the B cells are taken from a patient who is infected with HIV. By way of example and not limitation, B cells to be employed in the presently disclosed subject matter can be derived from subjects with cancer. It will be appreciated by one of ordinary skill in the art that B cells can be derived from any subject, and particularly those subjects having or possessing an antigen of interest, or those subjects infected with a pathogen for which an antigen is of interest.

II.A. EBV Transformation of B Cells

Epstein-Barr virus (EBV) is a large double-stranded DNA-containing herpes virus capable of infecting human B lymphocytes and epithelial cells (Kieff and Rickinson, 2006). Epstein-Barr virus, an oncogenic herpesvirus, infects nearly 90% of adults worldwide. Despite the high prevalence of infection, EBV-associated malignancies are largely kept in check by a strong cytotoxic T cell immune response. However, immune suppression, such as after transplant or during HIV infection can lead to the proliferation of EBV-infected B cells that can drive lymphomagenesis. EBV is causally implicated in the pathogenesis of post-transplant lymphoproliferative disease, HIV-associated CNS lymphomas, and nasopharyngeal carcinoma. Furthermore, it is a co-factor in nearly all cases of African endemic Burkitt's lymphoma and present in nearly half of sporadic Hodgkin's disease cases. Importantly, EBV transforms primary B lymphocytes in vitro into indefinitely proliferating lymphoblastoid cell lines (LCLs). LCLs express a subset of viral gene products similar to that found in EBV-associated cancers. Thus, LCLs represent a viable model for the pathogenesis of EBV-associated malignancies.

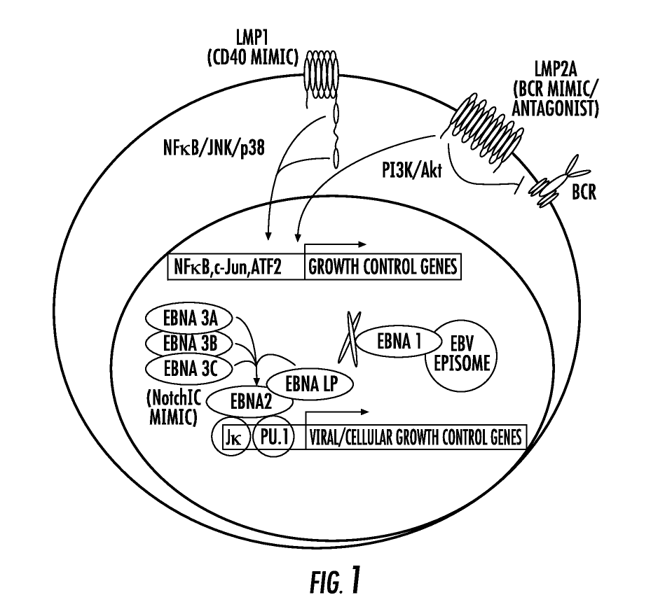

While epithelial cell infection typically results in lytic replication, infection of B cells leads to a latent infection where only a subset of the genome is expressed (Kieff and Rickinson, 2006). This gene expression program, termed latency III, drives resting B cells into the cell cycle, usurps core B cell signaling functions, and prevents cell death (FIG. 1).

While infection of primary B cells by EBV can lead to immortalization, the efficiency of this process is typically in the range of 1-10% of infected cells. Nearly all infected cells express viral latent genes. However, a block to long-term proliferation exists in the majority of these cells. The nature and molecular basis for this block is first disclosed herein.

Epstein-Barr virus nuclear antigen leader protein, or EBNA-LP, and EBNA2 are the first viral proteins expressed following primary B cell infection (Alfieri et al., 1991). Within one day following infection, EBNA2 and EBNA-LP transcriptionally upregulate a variety of viral and cellular genes, inducing a transition of resting B cells into the cell cycle (Sinclair et al., 1994; Wang et al., 1991; Wang et al., 1990). The initial viral targets of EBNA2 are the remaining EBNA genes: EBNA1, EBNA3A, EBNA3B, and EBNA3C (Zimber-Strobl et al., 1993; Zimber-Strobl et al., 1991). Concurrently, cellular genes are induced including the S phase initiating proto-oncogenes c-Myc, E2F1, and cyclin D2 (Kaiser et al., 1999; Sinclair et al., 1994). Within three days, the viral latent membrane proteins, LMP1 and LMP2A/2B, are induced by EBNA2 from a distinct promoter and the infected cells have initiated DNA synthesis (Wang et al., 1990) (Tsang et al., 1991). Latent virus replication is linked to cellular proliferation in that the viral episome is maintained and faithfully replicated during S phase by EBNA1 (Yates et al., 1985). Little to no virus particle production occurs in these cells (Alfieri et al., 1991). Over the course of the first week of infection, the viral proteins approach steady-state levels similar to that found in LCLs (Alfieri et al., 1991).

EBV infection in vivo has important similarities and differences with that found in vitro (Thorley-Lawson and Gross, 2004). Initial infection occurs in resting naïve B cells in the oral mucosa (Joseph et al., 2000). The infected cell enters latency III which drives proliferation. These cells then either enter germinal centers (Roughan and Thorley-Lawson, 2009) or mimic germinal center behavior by viral expression of BCR and CD40 mimics LMP2A and LMP1, respectively. Following this activation, EBV-infected B cells are retained in a default program of latency in long-lived memory cells in the periphery (Babcock and Thorley-Lawson, 2000; Souza et al., 2005). These cells express no viral proteins, except presumably EBNA1 prior to cell division. The use of the normal B cell maturation pathway in vivo allows EBV to gain access to a long-lived cell reservoir that defines its life in the periphery of nearly every infected individual.

In some embodiments of the presently disclosed subject matter B cells are growth transformed in vitro with EBV. As used herein, EBV transformation of B cells is also referred to as activation of B cells. In addition to transformation, activation of B cells via EBV infection can also increase B cell proliferation. In some embodiments, B cell EBV-mediated activation or transformation results in immortalization of B cells.

As an alternative to using EBV, other equivalent lymphocyte transforming agents can be used, including other viruses that can transform B cells, without departing from the scope of the presently disclosed subject matter. EBV is suitable for transforming the B cells of most primates but, for other organisms, suitable viruses can be selected as would be appreciated by those of skill in the art.

Methods of infecting B cells with EBV are known to those of skill in the art. In some embodiments B cells can be infected with EBV as disclosed in the Examples below. In some embodiments primary human peripheral blood mononuclear cells (PBMCs) can be infected with the EBV strain B95-8 with dilutions starting from a multiplicity of infection such that all B cells express the EBV latent gene expression program (approximately 100 particles/cell) through a dilution where no LCLs grow out. Culturing of the EBV-infected cells in RPMI 1640, 10% FCS, antibiotics, and in the presence of cyclosporine to suppress T cell activity, allows for the outgrowth of LCLs at approximately 1-5% efficiency (transformant/infected cell) within 5 weeks. Scoring of LCL outgrowth can be performed using techniques and analytical equipment as is know to those of skill in the art. By way of example and not limitation, scoring of LCL outgrowth can be performed using a Cellomics Vti Array Scan instrument (ThermoFisher Scientific, Waltham, Mass., United States of America) following Calcein-AM live cell fluorescent staining, which allows for an unbiased readout of LCL outgrowth. Alternatively, manual inspection of LCL outgrowth at 5 weeks can be used to calculate transformation efficiency.

In some embodiments of the presently disclosed subject matter the EBV transformation efficiency of B cells can be markedly improved by employing the methods disclosed herein. In particular, EBV transformation efficiency of B cells can be increased by concomitantly inhibiting host innate responses to activation-mediated proliferative signals, as discussed further herein. In some embodiments, EBV transformation efficiency of B cells can be increased by about 5%, 10%, 15%, 20%, 30%, 40%, 50%, 60%, 70%, 80%, 90%, 95%, 96%, 97%, 98%, 99%, or 100%, relative to EBV transformation without inhibiting host innate responses to activation-mediated proliferative signals.

The presently disclosed subject matter in some embodiments provides a method for producing immortalized B cells by coupling EBV transformation of the B cells with inhibitition of host innate responses to activation-mediated proliferative signals. Upon immortalization, B cells can be screened to select cells that produce antibodies with a desired specificity. Selected cells can then be used for monoclonal antibody production.

II.B. Polyclonal Activators of B Cells

In some embodiments of the presently disclosed subject matter the B cell activator can comprise a polyclonal activator. As used in the presently disclosed subject matter, the term “polyclonal activator” refers to a molecule or compound or a combination thereof that activates B cells irrespective of their antigenic specificity. As will be appreciated by one of ordinary skill in the art, a range of different molecules can be used as a polyclonal activator without departing from the scope of the presently disclosed subject matter.

By way of example and not limitation, polyclonal activators of the presently disclosed subject matter can comprise an agonist of a Toll Like Receptor (TLR), which is expressed on B cells. Exemplary TLRs that are expressed on memory B cells include but are not limited to TLR-7, TLR-9 and TLR-10 (Bernasconi et al. 2003). TLRs are pattern recognition receptors of the innate immune system expressed on a variety of cells including dendritic cells and B cells (Medzhitov & Janeway 2000, 2002). TLR agonists include microbial products and synthetic compounds. In some embodiments, a TLR agonist to be employed in the presently disclosed subject matter can be selected from the group comprising agonists of TLR-7, TLR-9, TLR-10 and combinations thereof. Such molecules can be of microbial or cellular origin or synthetic. In some embodiments, the activator can comprise an antibody, small molecule, or nucleic acid mimic (such as but not limited to an aptamer).

In some embodiments, a polyclonal activator employed in the presently disclosed methods can comprise a CpG oligodeoxynucleotide, R-848, CD40L, BAFF, cells that express CD40L or BAFF, and monoclonal antibodies that mimic one or more effects of the foregoing activators.

Unmethylated DNA oligonucleotides (also known as CpG) can be TLR-9 agonists. CpG can stimulate dendritic cell maturation and activate B cell proliferation and differentiation polyclonally, i.e. irrespective of the antibody specificity (Krieg et al. 1995; Krieg 2002). In some embodiments, the biological effect of CpG is dependent on specific sequences and chemical modifications (Krieg 2002). CpG oligonucleotides can be used as polyclonal activators, and examples of suitable activators are CpGs such as CpG 2006 (SEQ ID NO. 1; Hartmann et al. 2000) and other oligonucleotide sequences that trigger TLR-9 or other TLRs. As used herein, “CpG” refers to a sequence of unmethylated DNA oligonucleotides. More particularly, the term “CpG” can comprise single-stranded DNA molecules of between 5 and 100 nucleotides in length (e.g. 10-80, 20-70, or 30-60 nucleotides in length) that include one or more instances (e.g. 2, 4, 6, 8, 10 or more) of the dinucleotide CG sequence, with the C in the dinucleotide being unmethylated.

Imidazoquinoline compounds, such as R-848 (resiquimod), trigger TLR-7 and TLR-8 and stimulate dendritic cell maturation (Hemmi et al. 2002). Such compounds can be used as polyclonal activators in accordance with the presently disclosed subject matter. The presently disclosed subject matter also provides for the use of analogs or functional derivates or fragments of R-848. Also provided by the presently disclosed subject matter are other synthetic compounds that trigger TLR-7 and TLR-8, including but not limited to imiquimod, loxoribine, and guanosine analogs (e.g. 7-thia-8-oxoguanosine and 7-deazaguanosine).

Other polyclonal activators provided for use in the presently disclosed subject matter include other agonists of TLRs and of other pattern recognition receptors (PRRs) that are expressed on B memory cells, including monoclonal antibodies specific for TLRs. Additional polyclonal activators include CD40L, B-cell activating factor (BAFF; also known as tumor necrosis factor superfamily member 13B, BLyS, or THANK; Schneider et al. 1999), antibodies specific for CD40 and other molecules expressed by dendritic cells and activated T cells. In some embodiments, the cells, e.g. dendritic and/or activated T cells, themselves can be used as polyclonal activators. In some embodiments, the culture supernatant from activated T cells or any other cells (e.g. LCLs) can be used as a polyclonal activator.

In some embodiments, polyclonal activators can also include pathogen-associated molecular patterns (PAMPs), such as lipopolysaccharide (LPS), peptidoglycans, flagellins, zymosans and other cell wall components found in pathogens. Other available polyclonal activators include loxoribine, heat-killed Acholeplasma ladilawii, heat-killed Listeria monocytogenes, lipoteichoic acids, tripalmitoylated lipopeptides (e.g. Pam₃CSK4), single-stranded RNA (Diebold et al. 2004; Heil et al. 2004), doouble-stranded RNA, poly (I:C), and bacterial DNAs. A detailed list of TLR agonists can be found in Takeda et al. (2003).

II.C. Mitogentic Activators of B Cells

In some embodiments of the presently disclosed subject matter the B cell activator can comprise a mitogenic activator. As used in the presently disclosed subject matter, the terms “mitogenic activator”, “mitogen”, and “B cell mitogen” are used interchangeably and refer to a molecule or compound or a combination thereof that activates B cells. As will be appreciated by one of ordinary skill in the art, a range of different molecules can be used as a mitogenic activator without departing from the scope of the presently disclosed subject matter.

By way of example and not limitation, mitogenic activators of the presently disclosed subject matter can comprise a B cell mitogen or combinations of B cell mitogens. In some embodiments, mitogenic activators comprise cytokines. In some embodiments, mitogenic activators can be selected from the group comprising IL-2, IL-4, IL-5, IL-10, IL-15, CD40L, a CD40 agonist, anti-Ig, and combinations thereof. In some embodiments, the mitogen is an antibody, small molecule, or nucleic acid mimic (such as but not limited to an aptamer) of the foregoing activators. In some embodiments, the culture supernatant from activated T cells or any other cells (e.g. LCLs) can be used as a polyclonal activator.

In some embodiments, mitogenic activators can be added during a transformation step to further enhance the efficiency of transformation. In some embodiments, mitogenic activators can be added during the immortalization step to further improve the efficiency of immortalization.

### III. Inhibition of Host Innate Response to Activator-Mediated Proliferative Signals

While the initial burst of viral and cell gene expression leads to the proliferation of infected cells in vitro, only a small percentage of infected cells will become indefinitely proliferating lymphoblasts (Henderson et al., 1977; Shannon-Lowe et al., 2005; Sugden and Mark, 1977). Prior to the instant disclosure, the mechanism that restricts long-term outgrowth in the majority of infected cells was not well understood. In particular, prior to the instant disclosure it was unclear whether the innate response to EBV-induced proliferation has any long-term functional consequence. Indeed, the study of EBV-mediated induction of innate tumor suppressor pathways, also referred to as host innate response to abberrent proliferative signals, has been limited.

The presently disclosed subject matter provides for the inhibition of host innate response to activator-mediated proliferative signals, which can include but is not limited to the DNA damage response (DDR), during EBV-mediated transformation of B cells. The presently disclosed subject matter also provides for the inhibition of host innate response to activator-mediated proliferative signals during activation of B cells with activators other than EBV. The presently disclosed subject matter demonstrates that host innate responses to proliferative signals are initiated when B cells are activated, or proliferation increased, by exposure to polyclonal activators and/or mitogenic activators. See, e.g. the Examples provided herein. Accordingly, similar to EBV transformation, the presently disclosed subject matter provides for the inhibition of host innate response to activator-mediated proliferative signals in conjunction with activation of B cells to achieve increased proliferation of B cells and in some embodiments production of antibodies.

Accordingly, the presently disclosed subject matter provides for the inhibition of a host innate response to activator-mediated proliferative signals during activation of B cells. In some embodiments the activation comprises EBV-mediated transformation of B cells. In some embodiments the activation comprises activation of B cells using polyclonal activators. In some embodiments activation of B cells comprises exposing the B cells to mitgonic activators. In some embodiments combinations of activators disclosed herein can be used to activate B cells. In some embodiments the activation comprises increased proliferation of the B cells. In some embodiments activation comprises immortalization of the B cells. In some embodiments, inhibition of a host innate response to activator-mediated proliferative signals during activation of B cells provides for enhanced activation, and/or increased proliferation, and/or improved EBV-mediated transformation efficiency, as compared to activation without inhibition of a host innate response to activator-mediated proliferative signals. In some embodiments, the host innate response to activator-mediated proliferative signals is DDR.

In the presently disclosed subject matter the phrases “host innate response to activator-mediated proliferative signals”, “host innate response to activator-mediated proliferative pathways”, “host innate response to abberrent proliferative signals”, “host innate response to abberrent proliferative pathways”, “innate tumor suppressor signals”, “innate tumor suppressor pathways”, and variants thereof are used interchangeably. In some embodiments a host innate response to activator-mediated proliferative signals comprises the DDR, partial pathways of the DDR, associated pathways of the DDR, and components thereof. In some embodiments, the host innate response to activator-mediated proliferative pathways comprises EBV-mediated proliferative pathways.

III.A. The DNA Damage Response

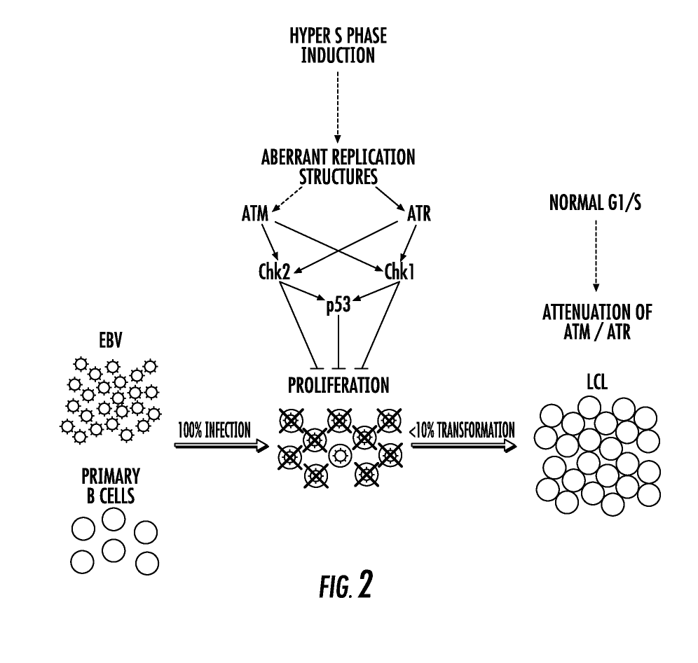

The DNA damage response (DDR) is a tumor suppressor pathway (FIG. 2) acting both in vitro and in vivo (Bartkova et al., 2005; DiTullio et al., 2002; Gorgoulis et al., 2005). The DDR responds to aberrant replication structures generated by activated oncogenes attempting to constitutively fire new origins and inappropriately enter S phase (Halazonetis et al., 2008). At least one function of the DDR is to limit aberrant proliferation by mediating oncogene-induced senescence and apoptosis (Bartkova et al., 2006; Di Micco et al., 2006; Mallette et al., 2007). Selective pressure to escape this early response to oncogenic stress leads to mutations in the DDR ultimately generating the large-scale chromosomal aberrations found in cancer (Halazonetis, 2004).

The response to genotoxic stress leads to complex signaling pathways that culminate in cell cycle arrest and DNA repair or, if the damage is too great, permanent cell cycle arrest or cell death (Bartek and Lukas, 2007). Cells activate the ATR signaling pathway in response to UV-irradiation or replicative stress (Paulsen and Cimprich, 2007). In the latter scenario, ssDNA exposed by collapsed replication forks leads to the accumulation of Replication protein A (RPA) and the signaling adaptors ATR interacting protein (ATRIP), Claspin, and TopBP1 that in turn activate ATR, its downstream effector Chk1, and a series of factors that limit further firing and progression of replication origins (Paulsen and Cimprich, 2007). In contrast, double-stranded DNA breaks at any point in the cell cycle are recognized by the Mre11/Rad50/Nbs1 (MRN) complex leading to activation of the ATM kinase, Chk2, and downstream targets regulating repair, cell cycle progression, and apoptosis (Lavin et al., 2005). Despite these clear biochemical differences, the two pathways overlap extensively dependent on the genotoxic stimulus. Indeed, ATM activation leads to ATR and Chk1 phosphorylation in response to ionizing radiation during S and G2 phases of the cell cycle (Jazayeri et al., 2006). Conversely, the activation of ATR in response to replicative stress during S phase can lead to ATM and Chk2 phosphorylation (Stiff et al., 2006). The DDR signal transduction pathway characterized thus far downstream of oncogenic stress involves both the replication checkpoint activating ATR/Chk1 pathway as well as the double-stranded break (DSB) activated ATM/Chk2 pathway (Bartkova et al., 2005; DiTullio et al., 2002; Gorgoulis et al., 2005). Additional DDR pathway intermediates, and potential targets, include DNA-PK, PARP family poly ADP-ribose polymerases, and Tip60 (acetyltransferas)/. There is also evidence that distinct oncogenes lead to distinct phosphorylation events (Mallette et al., 2007; Nuciforo et al., 2007) likely resulting from non-equivalent stressors (either DNA structures, levels of damage, or additional consequences of oncogene over-expression) and cell-type specific differences. Nevertheless, in a growing number of model oncogenic systems the activation of a DDR downstream of aberrant replication is emerging as playing a role in tumor suppression (Bartkova et al., 2006; Pusapati et al., 2006).

III.B. Inhibitors of Host Innate Response to Activator-Mediated Proliferative Signals

The presently disclosed subject matter provides for the use of inhibitors of host innate response to activator-mediated proliferative signals in conjunction with B cell activation. Inhibitors of host innate response to activator-mediated proliferative signals can comprise inhibitors of molecules, compounds or intermediates of any host innate response to activator-mediated proliferative signals. In some embodiments, the inhibitors can comprise small molecule inhibitors of one or more components of a pathway of a host innate response to activator-mediated proliferative signals. In some embodiments, the inhibitors can comprise inhibitors of DDR, and particularly inhibitors of DDR pathway intermediates or components, e.g. checkpoint kinases. In some embodiments, the inhibitors employed in the presently disclosed subject matter comprise inhibitors of kinase checkpoints in the DDR. In some embodiments of the presently disclosed subject matter the inhibitors employed in the presently disclosed subject matter comprise inhibitors of the ATR/Chk1 pathway, or inhibitors of the ATM/Chk2 pathway. In some embodiments the inhibitors employed in the presently disclosed subject matter comprise inhibitors of ATM, ATR, Chk1, Chk2, or DNA-PK, PARP family poly ADP-ribose polymerases, Tip60 (acetyltransferase), or combinations thereof.

By way of example and not limitation, an ATM inhibitor of the presently disclosed subject matter is ATM inhibitor (ATMi) KU-55933 (Hickson et al., 2004 Cancer Research 64:9152-9159), the structure of which is as follows:

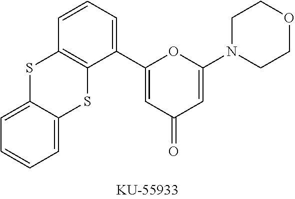

By way of example and not limitation, a Chk2 inhibitor of the presently disclosed subject matter is Chk2 inhibitor (Chk2i) II (Arienti et al., 2005 J. Med Chem 48:1873-1875), the structure of which is as follows:

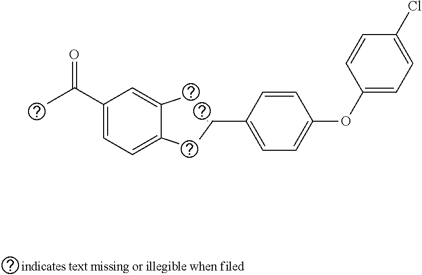

By way of example and not limitation, a PARP inhibitor of the presently disclosed subject matter is PARP inhibitor III DPQ (Suto et al., 1991 Anticancer Drug Des. 6:107). By way of example and not limitation, a DNA-PK inhibitor of the presently disclosed subject matter is DNA-PK inhibitor NU7026 (Hollick et al., 2003 Bioorg. Med. Chem. Lett. 13:3083-3086). Other existing and/or commercially available DDR inhibitors as would be known to one of ordinary skill in the art can be employed without departing from the scope of the presently disclosed subject matter.

In some embodiments, an inhibitor of a host innate response to activator-mediated proliferative signals can comprise any nucleic acid molecule capable of mediating RNA interference (RNAi) or gene silencing. In some embodiments a nucleic acid molecule capable of mediating RNAi or gene silencing can be provided to modulate or silence the expression of a gene encoding an intermediate in a host innate response to activator-mediated proliferative signals pathway, e.g. DDR. In some embodiments a nucleic acid molecule capable of mediating RNAi or gene silencing can be directed the ATR/Chk1 pathway or ATM/Chk2 pathway of the DDR. In some embodiments a nucleic acid molecule capable of mediating RNAi or gene silencing can be directed to ATM, ATR, Chk1, Chk2, or DNA-PK, PARP family poly ADP-ribose polymerases, Tip60 (acetyltransferase), or combinations thereof.

As would be appreciated by one of ordinary skill in the art, a nucleic acid molecule capable of mediating RNAi or gene silencing can comprise a small interfering RNA (siRNA), short interfering RNA (siRNA), microRNA (miRNA), short hairpin RNA (shRNA), or the like.

The terms “short hairpin RNA” and “shRNA” are used interchangeably and refer to any nucleic acid molecule capable of generating siRNA. In one embodiment, the shRNA comprises a polynucleotide having one or more loop structures and a stem comprising self complementary sense and antisense regions, wherein the antisense region comprises a sequence complementary to a region of a target nucleic acid molecule, and wherein the polynucleotide can be processed either in vivo or in vitro to generate an active siRNA capable of mediating RNAi. In another embodiment, retroviral vectors encode shRNA, which are processed intracellularly, to generate siRNA that silence the expression of a target gene, such as a gene encoding an intermediate of DDR, for example.

The terms “small interfering RNA”, “short interfering RNA” and “siRNA” are used interchangeably and refer to any nucleic acid molecule capable of mediating RNA interference (RNAi) or gene silencing. See e.g., Bass, Nature 411:428-429, 2001; Elbashir et al., Nature 411:494-498, 2001a; and PCT International Publication Nos. WO 00/44895, WO 01/36646, WO 99/32619, WO 00/01846, WO 01/29058, WO 99/07409, and WO 00/44914. In one embodiment, the siRNA comprises a double stranded polynucleotide molecule comprising complementary sense and antisense regions, wherein the antisense region comprises a sequence complementary to a region of a target nucleic acid molecule (for example, an mRNA encoding an intermediate of the DDR). In another embodiment, the siRNA comprises a single stranded polynucleotide having self-complementary sense and antisense regions, wherein the antisense region comprises a sequence complementary to a region of a target nucleic acid molecule. As used herein, siRNA molecules need not be limited to those molecules containing only RNA, but further encompass chemically modified nucleotides and non-nucleotides.

The terms “microRNA” and “miRNA” refer are used interchangeably and refer to synthetic or single-stranded RNA molecules of 21-23 nucleotides in length, which regulate gene expression. The terms “miRNA” and “non-coding RNA” can be used interchangeably. Primary transcript (pri-miRNA) is processed to give rise to short-stem-loop pre-miRNA, which are further processed to produce miRNA, which are single-stranded RNA molecules of 21-23 nucleotides. The miRNA are partially complementary to one or several mRNA transcripts, and they downregulate expression of genes encoded by the transcripts with which they interact. Thus, synthetic miRNA that interact with a gene that encodes for an intermediate of DDR, for example, can be generated and used to effect the downregulation of the expression of an intermediate of DDR thereby inhibiting DDR.

The term “gene expression” generally refers to the cellular processes by which a biologically active polypeptide is produced from a DNA sequence and exhibits a biological activity in a cell. As such, gene expression involves the processes of transcription and translation, but also involves post-transcriptional and post-translational processes that can influence a biological activity of a gene or gene product. These processes include, but are not limited to RNA syntheses, processing, and transport, as well as polypeptide synthesis, transport, and post-translational modification of polypeptides. Additionally, processes that affect protein-protein interactions within the cell can also affect gene expression as defined herein.

The term “modulate” refers to a change in the expression level of a gene, or a level of RNA molecule or equivalent RNA molecules encoding one or more proteins or protein subunits, or activity of one or more proteins or protein subunits is up regulated or down regulated, such that expression, level, or activity is greater than or less than that observed in the absence of the modulator. For example, the term “modulate” can mean “inhibit” or “suppress”, but the use of the word “modulate” is not limited to this definition.

As used herein, the terms “silence”, “ablate”, “inhibit”, “suppress”, “downregulate”, “loss of function”, “block of function”, and grammatical variants thereof are used interchangeably and refer to an activity whereby gene expression (e.g., a level of an RNA encoding one or more gene products) is reduced below that observed in the absence of a composition of the presently disclosed subject matter. In some embodiments, inhibition results in a decrease in the steady state level of a target RNA. In some embodiments, inhibition results in an expression level of a gene product that is below that level observed in the absence of the modulator.

In some embodiments, the terms “inhibit”, “suppress”, “downregulate”, “block of function” and grammatical variants thereof refer to a biological activity of a polypeptide or polypeptide complex that is lower in the presence of a modulator than that which occurs in the absence of the modulator. For example, a modulator can inhibit expression or function of an intermediate of the DDR.

As used herein, the terms “gene” and “target gene” refer to a nucleic acid that encodes an RNA, for example, nucleic acid sequences including, but not limited to, structural genes encoding a polypeptide. The target gene can be a gene derived from a cell, an endogenous gene, a transgene, etc. The cell containing the target gene can be derived from or contained in any organism, for example an animal. The term “gene” also refers broadly to any segment of DNA associated with a biological function. As such, the term “gene” encompasses sequences including but not limited to a coding sequence, a promoter region, a transcriptional regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

As is understood in the art, a gene comprises a coding strand and a non-coding strand. As used herein, the terms “coding strand” and “sense strand” are used interchangeably, and refer to a nucleic acid sequence that has the same sequence of nucleotides as an mRNA from which the gene product is translated. As is also understood in the art, when the coding strand and/or sense strand is used to refer to a DNA molecule, the coding/sense strand includes thymidine residues instead of the uridine residues found in the corresponding mRNA. Additionally, when used to refer to a DNA molecule, the coding/sense strand can also include additional elements not found in the mRNA including, but not limited to promoters, enhancers, and introns. Similarly, the terms “template strand” and “antisense strand” are used interchangeably and refer to a nucleic acid sequence that is complementary to the coding/sense strand.

The term “nucleic acid” refers to deoxyribonucleotides or ribonucleotides and polymers thereof in either single or double stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed base and/or deoxyinosine residues (Batzer et al. (1991) Nucleic Acid Res 19:5081; Ohtsuka et al. (1985) J Biol Chem 260:2605 2608; Rossolini et al. (1994) Mol Cell Probes 8:91 98). The terms “nucleic acid” or “nucleic acid sequence” can also be used interchangeably with gene, open reading frame (ORF), cDNA, and mRNA encoded by a gene.

The term “RNA” refers to a molecule comprising at least one ribonucleotide residue. By “ribonucleotide” is meant a nucleotide with a hydroxyl group at the 2′ position of a D-ribofuranose moiety. The terms encompass double stranded RNA, single stranded RNA, RNAs with both double stranded and single stranded regions, isolated RNA such as partially purified RNA, essentially pure RNA, synthetic RNA, recombinantly produced RNA, as well as altered RNA, or analog RNA, that differs from naturally occurring RNA by the addition, deletion, substitution, and/or alteration of one or more nucleotides. Such alterations can include addition of non-nucleotide material, such as to the end(s) of an siRNA or internally, for example at one or more nucleotides of the RNA. Nucleotides in the RNA molecules of the presently disclosed subject matter can also comprise non-standard nucleotides, such as non-naturally occurring nucleotides or chemically synthesized nucleotides or deoxynucleotides. These altered RNAs can be referred to as analogs or analogs of a naturally occurring RNA.

### IV. B Cell Activation Coupled With Inhibition of Host Innate Response to Activator-Mediated Proliferative Signals

The presently disclosed subject matter provides for the activation of primary B cells coupled with the inhibition of a host innate response to activator-mediated proliferative signals to optimize B cell proliferation. In optimizing B cell proliferation by activating B cells and inhibiting a host innate response to activator-mediated proliferative signals the presently disclosed subject matter provides in some embodiments for the rapid isolation of large numbers of human monoclonal antibodies from the memory repertoire with no need for specific immunization or boosting. In some embodiments B cell activation comprises EBV transformation. In some embodiments B cell activation comprises exposing B cells to one or more polyclonal activators or mitogenic cytokines, or combinations thereof. In some embodiments inhibition of a host innate response to activator-mediated proliferative signals comprises inhibiting the DNA damage response (DDR) pathway.

In contrast to existing methods of activating B cells (e.g., PCT Publication No. WO/2004/076677), the presently disclosed subject matter in some embodiments couples the activation of B cells with the inhibition of a host innate response to activator-mediated proliferative signals. Using this approach, a normal host suppressive pathway is liberated such that B cell proliferation is increased, antibody production is increased, and EBV-mediated transformation efficiency is markedly improved. This approach has the ability to produce robust EBV transformants with antibody producing capacity. Further, the inhibitors of the presently disclosed subject matter are relatively inert with respect to resting B cell function. Therefore, using the approach of the presently disclosed subject matter, negative consequences such as potential down-modulation of immunoglobulin expression level or other unwanted aspects of polyclonal activation with respect to the antibody-producing cells can be avoided.

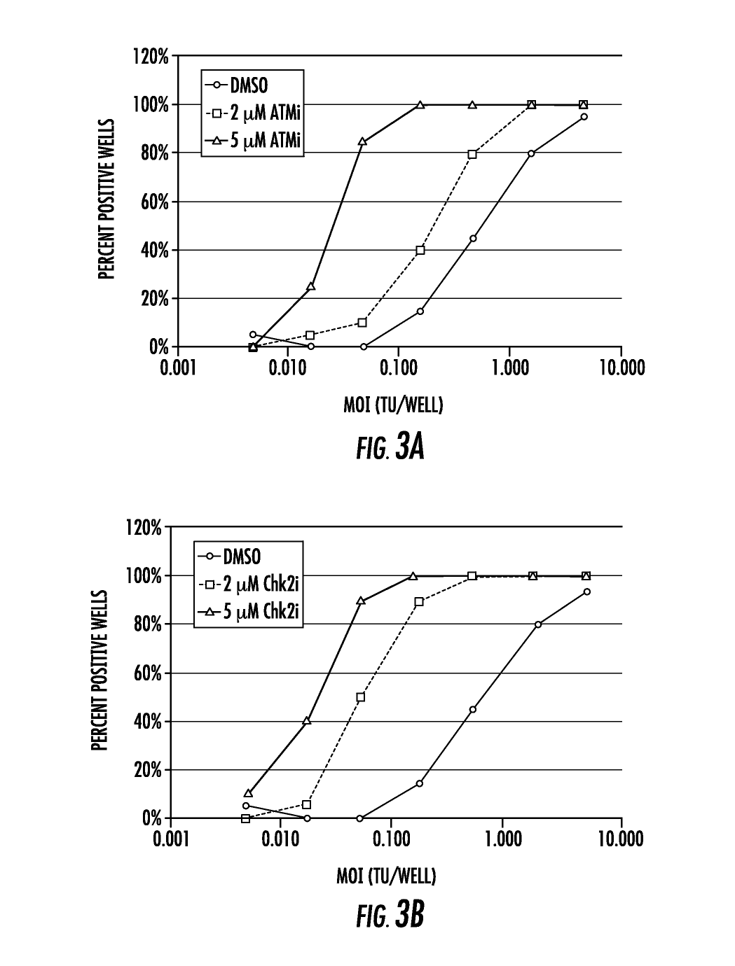

In some embodiments, coupling the activation of primary B cells with the inhibition of a host innate response to activator-mediated proliferative signals comprises incubating B cells in vitro with an activator, e.g. EBV, together with an inhibitor of host innate response to activator-mediated proliferative signals, e.g. a DDR inhibitor. By way of example and not limitation, incubating B cells with EBV, i.e. infection B cells with EBV, concomitant with 5 μM ATM or Chk2 inhibitor increases B cell transformation efficiency between 5-30 fold (see FIGS. 3A and 3B).

In some embodiments of the presently disclosed subject matter, coupling the activation of B cells with the inhibition of host innate response to activator-mediated proliferative signals provides for the immortalization of primary B cells. In some embodiments the immortalization of primary B cells comprises transforming primary B cells with EBV while exposing the B cells to an inhibitor of host innate response to EBV-mediated proliferative signals. In some embodiments, immortalized B cells can provide for the production of B cell clones capable of producing monoclonal antibodies with a desired antigen specificity.

In some embodiments of the presently disclosed subject matter, coupling the activation of B cells with the inhibition of host innate response to activator-mediated proliferative signals provides for increased efficiency of EBV transformation of primary B cells. In some embodiments the immortalization of primary B cells comprises transforming primary B cells with EBV while exposing the B cells to an inhibitor of host innate response to EBV-mediated proliferative signals. In some embodiments, the EBV transformation efficiency of B cells can be markedly improved compared to EBV transformation without inhibition of host innate response to EBV-mediated proliferative signals. In some embodiments, the efficiency of EBV transformation is expressed as the percentage of cells exposed to EBV for transformation to those that were actually transformed. In some embodiments, EBV transformation efficiency of B cells according to the presently disclosed subject matter can be increased by about 5%, 10%, 15%, 20%, 30%, 40%, 50%, 60%, 70%, 80%, 90%, 95%, 96%, 97%, 98%, 99%, or 100%, relative to EBV transformation without inhibiting host innate responses to activation-mediated proliferative signals. In some embodiments, the efficiency of EBV transformation achieved by the methods of the presently disclosed subject matter can be about 5%, 10%, 15%, 20%, 30%, 40%, 50%, 60%, 70%, 80%, 90%, 95%, 96%, 97%, 98%, 99%, or 100%.

In some embodiments of the presently disclosed subject matter, coupling the activation of B cells with the inhibition of host innate response to activator-mediated proliferative signals provides for increased proliferation of primary B cells in culture. In some embodiments the activator can comprise a polyclonal activator, a mitogenic activator, EBV, or combinations thereof.

In some embodiments the polyclonal activator is a CpG oligodeoxynucleotide. In some embodiments the inhibitor of host innate response to activator-mediated proliferative signals is an inhibitor of DDR. In some embodiments, the proliferation of B cells according to the presently disclosed subject matter can be increased by about 5%, 10%, 15%, 20%, 30%, 40%, 50%, 60%, 70%, 80%, 90%, 95%, 96%, 97%, 98%, 99%, or 100%, relative to B cell activation and proliferation without inhibiting host innate responses to activation-mediated proliferative signals.

In some embodiments of the presently disclosed subject matter, coupling the activation of B cells with the inhibition of host innate response to activator-mediated proliferative signals provides for the production of monoclonal antibodies. In some embodiments the activator can comprise a polyclonal activator, a mitogenic activator, EBV, or combinations thereof. In some embodiments the polyclonal activator is a CpG oligodeoxynucleotide. In some embodiments the inhibitor of host innate response to activator-mediated proliferative signals is an inhibitor of DDR. In some embodiments, the production of monoclonal antibodies from activated B cells according to the presently disclosed subject matter can be increased by about 5%, 10%, 15%, 20%, 30%, 40%, 50%, 60%, 70%, 80%, 90%, 95%, 96%, 97%, 98%, 99%, or 100%, relative to the production of monoclonal antibodies from B cells without B cell activation coupled with inhibiting host innate responses to activation-mediated proliferative signals.

Antibodies produced in accordance with the presently disclosed subject matter are selected from the physiological immunocompetent environment stimulated by natural contact with a pathogen or antigen. The methods of the presently disclosed subject matter are therefore particularly useful to produce antibodies against antigenic determinants that are specifically recognized by a desired subject's (e.g., human) immune system. These include neutralizing antibodies to pathogens and antibodies to allergens, tumor antigens, auto-antigens and allo-antigens that are part of the memory repertoire of a given subject. In some embodiments, the antibodies of the presently disclosed subject matter are fully human and exploit the diversity generated in the course of a human immune response. It will be appreciated by those of skill in the art that the methods of the presently disclosed subject matter can be used for the production of antibodies with specificity for any antigen that is present in the human memory repertoire. Additionally, the presently disclosed subject matter provides for the production of monoclonal antibodies with a desired antigen specificity.

### V. Applications of Presently Disclosed Subject Matter

V.A. Screening and Isolation of B Cells

In some embodiments, transformed and/or activated B cells can be screened for those having the desired antigen specificity, and individual B cell clones can then be produced from the positive cells. The screening step can be carried out by ELISA, by staining of tissues or cells (including transfected cells), a neutralization assay, and/or one of a number of other methods known in the art for identifying desired antigen specificity. The assay can select on the basis of simple antigen recognition, or can select on the additional basis of a desired function, e.g. neutralizing antibodies rather than just antigen-binding antibodies, antibodies that can change characteristics of targeted cells, such as their signalling cascades, their shape, their growth rate, their capability of influencing other cells, their response to the influence by other cells, or by other reagents or by a change in conditions, their differentiation status, etc.

In some embodiments, a cloning step for separating individual clones from the mixture of positive cells can be carried out using limiting dilution, micromanipulation, single cell deposition by cell sorting, and/or by any other method known in the art. In some embodiments, cloning is carried out using limiting dilution. In some embodiments, cloned B cells are derived from B cells that have been immortalized using EBV-transformation coupled with inhibition of host innate response to activator-mediated proliferative signals.

In some embodiments, the presently disclosed subject matter provides for the production of immortalized B cells that produce antibodies having a desired antigen specificity. The present disclosure thus provides in some embodiments an immortalized B cell clone obtained by the methods of the presently disclosed subject matter. As would be appreciated by one of ordinary skill in the art and as described herein, these B cells can be used in various ways, e.g. as a source of monoclonal antibodies, as a source of nucleic acid (DNA or mRNA) encoding a monoclonal antibody of interest, for delivery to subjects for cellular therapy, as a therapeutic or pharmaceutical, or for research.

In some embodiments, the presently disclosed subject matter provides a composition comprising immortalized B cells, wherein the B cells produce antibodies, wherein the antibodies are produced at ≧10ⁿ ng per clone per day, wherein the value of n is selected from −3, −2, −1, 0, 1, 2, 3 or 4. In some embodiments the presently disclosed subject matter also provides a composition comprising clones of an immortalized B cell, wherein the clones produce a monoclonal antibody of interest, wherein the antibody is produced at ≧10ⁿ ng per clone per day, wherein the value of n is selected from −3, −2, −1, 0, 1, 2, 3 or 4.

V.B. Identification of Novel Monoclonal Antibodies

The presently disclosed subject matter can be used for the identification of novel antibodies. In some embodiments, the presently disclosed subject matter provides a method for identifying an antibody having a desired antigen specificity, comprising the steps of: (i) providing a subject infected with or possessing the antigen for which the antibody specificity is desired; (ii) culturing primary B cells obtained from the subject; (iii) providing to the cultured primary B cells an activator of B cell proliferation in the presence of an inhibitor of a host innate response to activator-mediated proliferative signals; and (iv) screening the supernatant from the culture of proliferating primary B cells for an antibody having the desired antigen specificity. In some embodiments, the antibody is a broadly neutralized antibody.

In some embodiments, the activator of B cells can be a polyclonal activator, mitogenic activator, EBV, or combinations thereof. In some embodiments, the inhibitor of a host innate response to activator-mediated proliferative signals can comprise an inhibitor of DDR, such as but not limited to a small molecule, shRNA, and the like, and singly or in combination.

In some embodiments, a method for identifying an antibody having a desired antigen specificity can further comprise (v) isolating from the culture a B cell clone capable of producing a neutralizing antibody having a desired antigen specificity; and (vi) culturing the B cell clone under conditions where the antibody having a desired antigen specificity is expressed.

In some embodiments, a method for identifying an antibody having a desired antigen specificity can further comprise (v) isolating from the culture a B cell clone capable of producing an antibody having a desired antigen specificity; (vi) obtaining and/or sequencing a nucleic acid for the antibody from the selected B cell clone; (vii) employing the nucleic acid to prepare an expression host that can express the antibody of interest; and (viii) culturing the expression host under conditions where the antibody having a desired antigen specificity is expressed.

In some embodiments, the supernatant from the activated B cells in culture can be screened for novel antibodies using known methods as would be known to those of skill in the art. Screening is performed to identify one or more monoclonal antibodies capable of binding to an antigen of interest. Generally, one of ordinary skill in the art would screen for antibodies which bind to an antigen of interest. Such screening can be performed on culture supernatant and/or purified antibodies. Alternatively, or in addition, screening can be, carried out using culture supernatant and/or purified antibodies from activated and/or immortalized B cells. In addition, where cross-reactive antibodies are of interest, the ability of the monoclonal antibodies to cross-react with two or more different antigens can be determined. Moreover, it can be desirable to screen for antibodies with certain functional characteristics (e.g. agonistic activity, blocking activity, etc).

The binding specificity of monoclonal antibodies produced by the presently disclosed subject matter can, for example, be determined in an immuno-assay, e.g. by immunoprecipitation or other in vitro binding assay, such as radioimmunoassay (RIA) or enzyme-linked immunoadsorbent assay (ELISA).

Three representative general classes of screening methods that can be employed (a) antibody capture assays; (b) antigen capture assays; and (c) functional screens. Combinations can also be employed.

In antibody capture assays, the antigen can be bound to a solid phase, monoclonal antibodies to be tested are allowed to bind to the antigen, unbound antibodies are removed by washing, and then the bound antibodies are detected, e.g. by a secondary reagent such as a labeled antibody that specifically recognizes the antibody.

For an antigen capture assay, the antigen can be labeled directly. In one embodiment, monoclonal antibodies to be tested can be bound to a solid phase and then reacted with the optionally labeled antigen. Alternatively, the antibody-antigen complex can be allowed to form by immunoprecipitation prior to binding of the monoclonal antibody to be tested to a solid phase. Once the antibody-antigen complexes are bound to the solid phase, unbound antigen can be removed by washing and positives can be identified by detecting the antigen.

Various functional screens exist for identifying monoclonal antibodies with desired activities. Examples include the agonistic activity assay and blocking assay; keratinocyte monolayer adhesion assay and the mixed lymphocyte response (MLR) assay (Werther et al. J. Immunol. 157:4986-4995 (1996)); tumor cell growth inhibition assays (as described in WO 89/06692, for example); antibody-dependent cellular-1-cytotoxicity (ADCC) and complement-mediated cytotoxicity (CDC) assays (U.S. Pat. No. 5,500,362); and hematopoiesis assays (see WO 95/27062). The class/subclass of the antibodies can be determined, e.g., by double-diffusion assays; antibody capture on antigen-coated plates; and/or antibody capture on anti-IgG antibodies.

To screen for antibodies which bind to a particular epitope on the antigen of interest, a routine cross-blocking assay such as that described in Antibodies, A Laboratory Manual, Cold Spring Harbor Laboratory, Ed Harlow and David Lane (1988), can be performed. Alternatively, epitope mapping, e.g., as described in Champe et al. (J. Biol. Chem. 270:1388-1394 (1995)) can be performed to determine whether the antibody binds an epitope of interest.

V.C. Characterization of B Cell Repertoire

In some embodiments, the presently disclosed subject matter provides methods for the characterization of a humoral repertoire, including but not limited to the full humoral repertoire, to an entity, such as a pathogen or vaccine. In some embodiments, methods are provided for collecting multiparametric datasets that describe the characteristics (e.g., the specificity, isotype, and apparent affinity) of the antibodies secreted from large numbers of individual primary B cells.

A humoral repertoire, including but not limited to the full humoral repertoire, to an entity, such as a pathogen or vaccine, can provide multi-dimensional information (e.g. specificities, affinities, stabilities, gene segment sequence preferences, etc) that could be considered a “profile” of a subject's humoral response. Quantitation of these parameters (Story et al., 2008 PNAS 105(46):17902-17907) may be used to correlate with protection from a pathogen or failure to protect. Such information could then inform vaccine design in an iterative fashion, provide the basis for a multi-parameter diagnostic assay for specific antigens, or be directly used to identify single or multiple neutralizing antibodies against a given pathogen.

In some embodiments, the presently disclosed subject matter provides a method for generating a profile of a humoral response of a subject, comprising the steps of: (i) culturing primary B cells obtained from a subject; (ii) providing to the cultured primary B cells an activator of B cell proliferation in the presence of an inhibitor of a host innate response to activator-mediated proliferative signals; (iii) screening the supernatant from the culture of proliferating primary B cells for one or more antibodies; and (iv) characterizing the one or more antibodies, wherein a profile of the humoral response of a subject is generated. In some embodiments characterizing the one or more antibodies comprises collecting multiparametric datasets that describe the characteristics, e.g. specificities, affinities, stabilities, isotyptes, gene segment sequence preferences, etc. (Story et al., 2008 PNAS 105(46):17902-17907) In some representative, non-limiting embodiments, the profile or multiparametric dataset can be used to inform vaccine design in an iterative fashion, provide the basis for a multi-parameter diagnostic assay for specific antigens, or be directly used to identify single or multiple neutralizing antibodies against a given pathogen.

V.D. Antigen Purification and Diagnostics

In some embodiments of the presently disclosed subject matter, antibodies can be particularly useful in identification and purification of an individual polypeptide or other antigen against which they are directed. The antibodies of the presently disclosed subject matter can have additional utility in that they can be employed as reagents in immunoassays, radioimmunoassays (RIA) or enzyme-linked immunosorbent assays (ELISA). In these applications, the antibodies can be labelled with an analytically-detectable reagent such as a radioisotope, a fluorescent molecule or an enzyme. The monoclonal antibodies produced by the methods disclosed herein can also be used for the molecular identification and characterization (epitope mapping) of antigens recognized by protected individuals in complex pathogens such as plasmodia, the isolation of cross-reactive protective antibodies in the case of highly variable pathogens such as those found in HIV, and for detecting pathogens and determining their variability.

Regarding diagnostic assays, antibodies of the presently disclosed subject matter can be employed in diagnostic assays for their antigen, e.g., detecting its expression in specific cells, tissues, or serum. Various diagnostic assay techniques known in the art can be used, such as competitive binding assays, direct or indirect sandwich assays and immunoprecipitation assays conducted in either heterogeneous or homogeneous phases (Zola, Monoclonal Antibodies: A Manual of Techniques, CRC Press, Inc. (1987) pp. 147-158). In some embodiments, the antibodies used in the diagnostic assays can be labeled with a detectable moiety. The detectable moiety should be capable of producing, either directly or indirectly, a detectable signal. For example, the detectable moiety can be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish petoxidase. Any method known in the art for conjugating the antibody to the detectable moiety can be employed, including those methods described by Hunter et al., Nature, 144:945 (1962); David et al., Biochemistry., 13:1014 (1974); Pain et al., J. Immunol. Meth., 40:219 (1981); and Nygren, J. Histochem. and Cytochem., 30:407 (1982). Detection of an antigen of interest using a diagnostic assay as described herein or as apparent to those skilled in the art upon a review of the present disclosure can provide for the diagnosis of a disease, condition or pathogen.

Antibodies produced in accordance with the presently disclosed subject matter are also useful for the affinity purification of antigen from recombinant cell culture or natural sources. In this process, the antibodies can be immobilized on a suitable support, such as Sephadex™ resin or filter paper, using methods well known in the art. The immobilized antibody then is contacted with a sample containing the antigen to be purified, and thereafter the support is washed with a suitable solvent that will remove substantially all the material in the sample except the antigen, which is bound to the immobilized antibody. Finally, the support is washed with another suitable solvent that will release the antigen from the antibody. This and other procedures as known to those of skill in the art can be employed to purify and/or isolate an antigen of interest using antibodies produced in accordance with the presently disclosed subject matter.

V.E. Pharmaceutical Compositions

The presently disclosed subject matter provides pharmaceutical compositions comprising the antibodies produced in accordance with the presently disclosed subject matter. In some embodiments, pharmaceutical compositions comprising transformed and/or activated B cells of the presently disclosed subject matter are provided. In some embodiments, a pharmaceutical composition can comprise one or more monoclonal antibodies produced in accordance with the presently disclosed subject matter. In some embodiments, both monoclonal antibodies as well as the transformed and/or activated B cells of the presently disclosed subject matter can be included in a pharmaceutical composition. In some embodiments, a panel or multitude of monoclonal antibodies produced by the presently disclosed subject matter can be included in a pharmaceutical composition.

Carriers. In some embodiments a pharmaceutical composition can also contain a pharmaceutically acceptable carrier or adjuvant for administration of the antibody. In some embodiments, the carrier is pharmaceutically acceptable for use in humans. The carrier or adjuvant should not itself induce the production of antibodies harmful to the individual receiving the composition and should not be toxic. Suitable carriers can be large, slowly metabolized macromolecules such as proteins, polypeptides, liposomes, polysaccharides, polylactic acids, polyglycolic acids, polymeric amino acids, ammo acid copolymers and inactive virus particles.

Pharmaceutically acceptable salts can be used, for example mineral acid salts, such as hydrochlorides, hydrobromides, phosphates and sulphates, or salts of organic acids, such as acetates, propionates, malonate and benzoates.

Pharmaceutically acceptable carriers in therapeutic compositions can additionally contain liquids such as water, saline, glycerol and ethanol. Additionally, auxiliary substances, such as wetting or emulsifying agents or pH buffering substances, can be present in such compositions. Such carriers enable the pharmaceutical compositions to be formulated as tablets, pills, dragees, capsules, liquids, gels, syrups, slurries and suspensions, for ingestion by the patient.

The compositions of the presently disclosed subject matter can further comprise a carrier to facilitate composition preparation and administration. Any suitable delivery vehicle or carrier can be used, including but not limited to a microcapsule, for example a microsphere or a nanosphere (Manome et al. (1994) Cancer Res 54:5408-5413; Saltzman & Fung (1997) Adv Drug Deliv Rev 26:209-230), a glycosaminoglycan (U.S. Pat. No. 6,106,866), a fatty acid (U.S. Pat. No. 5,994,392), a fatty emulsion (U.S. Pat. No. 5,651,991), a lipid or lipid derivative (U.S. Pat. No. 5,786,387), collagen (U.S. Pat. No. 5,922,356), a polysaccharide or derivative thereof (U.S. Pat. No. 5,688,931), a nanosuspension (U.S. Pat. No. 5,858,410), a polymeric micelle or conjugate (Goldman et al. (1997) Cancer Res 57:1447-1451 and U.S. Pat. Nos. 4,551,482, 5,714,166, 5,510,103, 5,490,840, and 5,855,900), and a polysome (U.S. Pat. No. 5,922,545).

Conjugation of Antibodies. Antibody sequences can be coupled to active agents or carriers using methods known in the art, including but not limited to carbodiimide conjugation, esterification, sodium periodate oxidation followed by reductive alkylation, and glutaraldehyde crosslinking (Goldman et al. (1997) Cancer Res. 57:1447-1451; Cheng (1996) Hum. Gene Ther. 7:275-282; Neri et al. (1997) Nat. Biotechnol. 15:1271-1275; Nabel (1997) Vectors for Gene Therapy. In Current Protocols in Human Genetics, John Wiley & Sons, New York; Park et al. (1997) Adv. Pharmacol. 40:399-435; Pasqualini et al. (1997) Nat. Biotechnol. 15:542-546; Bauminger & Wilchek (1980) Meth. Enzymol. 70:151-159; U.S. Pat. No. 6,071,890; and European Patent No. 0 439 095).

Formulation. A therapeutic composition, a diagnostic composition, or a combination thereof, of the presently disclosed subject matter comprises in some embodiments a pharmaceutical composition that includes a pharmaceutically acceptable carrier. Suitable formulations include aqueous and non-aqueous sterile injection solutions which can contain anti-oxidants, buffers, bacteriostats, bactericidal antibiotics and solutes which render the formulation isotonic with the bodily fluids of the intended recipient; and aqueous and non-aqueous sterile suspensions which can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are SDS in the range of in some embodiments 0.1 to 10 mg/ml, in some embodiments about 2.0 mg/ml; and/or mannitol or another sugar in the range of in some embodiments 10 to 100 mg/ml, in some embodiments about 30 mg/ml; and/or phosphate-buffered saline (PBS). Any other agents conventional in the art having regard to the type of formulation in question can be used. In some embodiments, the carrier is pharmaceutically acceptable. In some embodiments the carrier is pharmaceutically acceptable for use in humans.

Pharmaceutical compositions of the presently disclosed subject matter can have a pH between 5.5 and 8.5, preferably between 6 and 8, and more preferably about 7. The pH can be maintained by the use of a buffer. The composition can be sterile and/or pyrogen free. The composition can be isotonic with respect to humans. Pharmaceutical compositions of the presently disclosed subject matter can be supplied in hermetically-sealed containers.

Pharmaceutical compositions can include an effective amount of one or more antibodies of the presently disclosed subject matter and/or one or more transformed or activated B cells of the presently disclosed subject matter. In some embodiments, a pharmaceutical composition can comprise an amount that is sufficient to treat, ameliorate, or prevent a desired disease or condition, or to exhibit a detectable therapeutic effect. Therapeutic effects also include reduction in physical symptoms. The precise effective amount for any particular subject will depend upon their size and health, the nature and extent of the condition, and therapeutics or combination of therapeutics selected for administration. The effective amount for a given situation is determined by routine experimentation and is within the judgment of one of ordinary skill in the art.

V.F. Treatment of a Subject

The antibodies of the presently disclosed subject matter or fragments thereof can be used for the treatment of disease, for the prevention of disease and/or for the diagnosis of disease. In some embodiments, the monoclonal antibodies of the presently disclosed subject matter can be administered to a subject in need thereof, in a pharmaceutical composition or medicament as described herein, to treat an infectious disease, cancer, inflammatory disease, autoimmune disease, and the like.

Antibodies produced in accordance with the presently disclosed subject matter can be selected to target a specific antigen based on the antigen specificity of the antibody. In some embodiments an antibody of the presently disclosed subject matter can be selected to target a receptor, protein, ligand, surface peptide, marker or unique identifying characteristic of a pathogen, antigen, tumor, cancerous cell, tissue or other desired target. In some embodiments an antibody can be coupled to or associated with a pharmaceutical compound or therapeutic agent, e.g. a chemotherapeutic, whereby the antibody acts to deliver therapeutic agent to the desired target to elicit a therapeutic response.

In some embodiments the antibodies of the presently disclosed subject matter can be administered in combination with other therapeutics or treatments. By way of example and not limitation, an antibody produced in accordance with the presently disclosed subject matter can be administered to a subject in combination with effective amounts of one or more other therapeutic agents or in conjunction with radiation treatment. Therapeutic agents provided include chemotherapeutics as well as immunoadjuvants and cytokines. The antibody can be administered sequentially or concurrently with the one or more other therapeutic agents. The amounts of antibody and therapeutic agent depend, for example, on what type of drugs are used, the cancer being treated, and the scheduling and routes of administration but would generally be less than if each were used individually.

In some embodiments, antibodies produced in accordance with the presently disclosed subject matter can be administered to a subject as a vaccine. In some embodiments, the vaccine can be used to develop passive immunity in a subject.

V.G. Administration

Suitable methods for administration of a therapeutic composition, a diagnostic composition, or combinations thereof of the presently disclosed subject matter include but are not limited to intravascular, subcutaneous, or intratumoral administration. Further, upon a review of the instant disclosure, it is understood that any site and method for administration can be chosen, depending at least in part on the species of the subject to which the composition is to be administered. For delivery of compositions to pulmonary pathways, compositions can be administered as an aerosol or coarse spray.

For therapeutic applications, a therapeutically effective amount of a composition of the presently disclosed subject matter is administered to a subject. A “therapeutically effective amount” is an amount of therapeutic composition sufficient to produce a measurable biological response (e.g., a cytotoxic response, or tumor regression). Actual dosage levels of active ingredients in a therapeutic composition of the presently disclosed subject matter can be varied so as to administer an amount of the active compound(s) that is effective to achieve the desired therapeutic response for a particular subject. The selected dosage level will depend upon a variety of factors including but not limited to the activity of therapeutic composition, formulation, the route of administration, combination with other drugs or treatments, tumor size and longevity, and the physical condition and prior medical history of the subject being treated. In some embodiments of the presently disclosed subject matter, a minimal dose is administered, and dose is escalated in the absence of dose-limiting toxicity. Determination and adjustment of a therapeutically effective dose, as well as evaluation of when and how to make such adjustments, are known to those of ordinary skill in the art.

For diagnostic applications, a detectable amount of a composition of the presently disclosed subject matter is administered to a subject. A “detectable amount”, as used herein to refer to a diagnostic composition, refers to a dose of such a composition that the presence of the composition can be determined in vivo or in vitro. A detectable amount will vary according to a variety of factors, including but not limited to chemical features of the agent being labeled, the detectable label, labeling methods, the method of imaging and parameters related thereto, metabolism of the labeled agent in the subject, the stability of the label (e.g. the half-life of a radionuclide label), the time elapsed following administration of an active agent and/or labeled antibody prior to imaging, the route of drug administration, the physical condition and prior medical history of the subject, and the size and longevity of the tumor or suspected tumor. Thus, a detectable amount can vary and can be tailored to a particular application. After study of the present disclosure, including the Appendix, it is within the skill of one in the art to determine such a detectable amount.

### VI. Antibodies

The presently disclosed subject matter provides for the production of antibodies, including for example monoclonal antibodies. In some embodiments monoclonal antibodies of the presently disclosed subject matter have a desired antigen specificity. In some embodiments, monoclonal antibodies are produced by B cells immortalized in accordance with the presently disclosed subject matter. In some embodiments, monoclonal antibodies are obtained from B cell clones produced by immortalizing B cells in accordance with the presently disclosed subject matter. In some embodiments, monoclonal antibodies are produced by increasing the proliferartion of B cells in culture by incubating the B cells with an activator in conjuction with an inhibitor of host innate response to activator-mediated proliferative signals. As would be appreciated by one of ordinary skill in the art and as disclosed herein, the monoclonal antibodies of the presently disclosed subject matter also comprise functional fragments, derivates, variants and portions thereof that retain the antigen-binding activity of the antibodies.

In some embodiments, antibodies of the presently disclosed subject matter can be characterized as antibodies that recognize self antigens or non-self antigens. In some embodiments, antibodies that recognize self antigens can be used to detect or treat diseases caused by aberrant gene expression, including but not limited to cancers, and by aberrant protein processing, including but not limited to Alzheimer's disease. Antibodies that recognize non-self antigens can be used to detect or treat infectious diseases and pathogens, including but not limited to parasitic, viral and bacterial infections. The presently disclosed subject matter can advantageously provide human antibodies that recognize antigens of interest where it has not previously been possible.

The production of antibodies according to the presently disclosed subject matter provides for antibodies with the characteristics of those produced in the course of a physiological human immune response, i.e. antibody specificities that can only be selected by the human immune system. By way of example and not limitation, such a response to human pathogens includes responses to HIV, the Plasmodium species that cause human malaria, human hepatitis B and C viruses, Measles virus, Ebola virus, the SARS virus, Pox virus, Bunyaviridae, Arenaviridae, Bornaviridae, Reoviridae (including rotaviruses and orbiviruses), Retroviridae (including HTLV-I, HTLV-II, HIV-1, HIV-2), Polyomaviridae, Papillomaviridae, Adenoviridae, Parvoviridae, Herpesviridae (including herpes simplex viruses 1 and 2, cytomegaloviruses, varicella-zoster virus, herpesviruses 6A, 6B and 7), Poxyiridae, and Hepadnaviridae (for an exhaustive list see Fields et al. 1996). In some embodiments, antibodies of the presently disclosed subject matter can possess the characteristics of those produced in the course of a response to environmental allergens generated in allergic patients, to prion proteins, to tumor antigens generated in tumor bearing patients, to self-antigens in patients with autoimmune diseases, and to amyloid proteins. These antibodies can be used as prophylactic or therapeutic agents upon appropriate formulation or as a diagnostic tool.

In relation to any particular pathogen, a “neutralizing antibody”, “broadly neutralizing antibody”, or “neutralizing monoclonal antibody”, all of which are used interchangeably herein, is one that can neutralize the ability of that pathogen to initiate and/or perpetuate an infection in a host. In some embodiments, monoclonal antibodies produced in accordance with the presently disclosed subject matter have neutralizing activity, wherein the antibody can neutralize at a concentration of 10⁻⁹M or lower (e.g. 10⁻¹⁰M, 10⁻¹¹M, 10⁻¹²M or lower).

In some embodiments, the presently disclosed subject matter provides for the production of a neutralizing monoclonal antibodies directed to a desired antigen. In some embodiments, a neutralizing monoclonal antibody of the presently disclosed subject matter can comprise a desired antigen specificity selected from the group consisting of but not limited to: a pathogen, allergen, tumor antigen, autoantigen, alloantigen, microbial pathogen, chemical or toxin. In some embodiments, the desired antigen specificity of a neutralizing antibody is against a pathogen selected from the group comprising: HIV, P. falciparium, P. vivax, P. malariae, P. ovale, hepatitis A, hepatitis B, hepatitis C, Measles virus, Ebola virus, Poxviruses, Influenza virus, H1N1 virus, Avian influenza virus, herpes simplex virus type 1 or type 2; SARS coronavirus, mumps virus, rubella virus, rabies virus, papillomavirus, vaccinia virus, varicella-zoster virus, variola virus, polio virus, rhinoviruses, respiratory syncytial virus, a human endogenous retroviruses, Dengue virus, Corynebacterium diphtheriae, Clostridium tetani, Clostridium botulinum, Bordetella pertussis, Haemophilus influenzae, Neisseria meningitidis, serogroup A, B, C, W135 and/or Y; Streptococcus pneumoniae, Streptococcus agalactiae, Streptococcus pyogenes, Staplhylococcus aureus, Bacillus anthracis, Moraxella catarrhalis, Chlaymdia trachomatis, Chlamydia pneumoniae, Yersinia pestis, Francisella tularensis, Salmonella species, Vibrio cholerae, toxic E. coli, and other viral or microbial pathogen.

In some embodiments, monoclonal antibodies of the presently disclosed subject matter have an antigen specificity against proteins expressed in tumors, in diseased cardiovascular cells, during inflammatory responses, in neurological disorders (e.g. amyloid proteins of Alzheimer's disease), in encephalopathies, etc. The presently disclosed subject matter can also provide monoclonal antibodies that recognize narcotic substances such as cocaine, heroin, benzoylecgonine, amphetamines, etc.

Antibodies of the presently disclosed subject matter can be immunogenic in non-human (or heterologous) hosts, e.g. in mice. In particular, antibodies of the presently disclosed subject matter can have an idiotope that is immunogenic in non-human hosts, but not in a human host. Antibodies of the presently disclosed subject matter for human can comprise those that cannot be obtained from hosts such as mice, goats, rabbits, rats, non-primate mammals, etc., and cannot be obtained by humanization or from xeno-mice.

The immunoglobulin molecules of the presently disclosed subject matter can be of any type (e.g., IgG, IgE, IgM, IgD, IgA and IgY), class (e.g., IgG1, IgG2, IgG3, IgG4, IgA1 and IgA2), or subclass of immunoglobulin molecule. In some embodiments, the antibodies are antigen-binding antibody fragments (e.g., human) and include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Antigen-binding antibody fragments, including single-chain antibodies, can comprise the variable region(s) alone or in combination with the entirety or a portion of the following: hinge region, CH1, CH2, and CH3 domains. Also included in the presently disclosed subject matter are antigen-binding fragments comprising any combination of variable region(s) with a hinge region, CH1, CH2, and CH3 domains.

The antibodies and fragments thereof of the presently disclosed subject matter can be from any animal origin including birds and mammals. For example, the antibodies can be human, murine (e.g., mouse and rat), donkey, sheep, rabbit, goat, guinea pig, camel, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example in, U.S. Pat. No. 5,939,598.

In another embodiment of the presently disclosed subject matter, an antibody library (for example, a library of scFv antibodies) can be used to perform the disclosed screening methods. Such a library can be constructed as set forth in U.S. Pat. Nos. 6,593,081; 6,225,447; 5,580,717; and 5,702,892, all incorporated by reference herein.

The term “antibody” as used herein refers to monoclonal antibodies (including agonist, antagonist, and blocking or neutralizing antibodies) and antibody compositions with polyepitopic specificity.

The term “monoclonal antibody” as used herein refers to an antibody obtained from a population of substantially homogeneous antibodies, i.e., the individual antibodies comprising the population are identical except for possible naturally-occurring mutations that can be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen.

The monoclonal antibodies herein include hybrid and recombinant antibodies produced by splicing a variable (including hypervariable) domain of an antibody with a constant domain, or a light chain with a heavy chain, or a chain from one species with a chain from another species, or fusions with heterologous proteins, regardless of species of origin or immunoglobulin class or subclass designation, as well as antibody fragments (e.g., Fab′, Fab′, F(ab′).sub.2, and Fv), so long as they exhibit the desired biological activity. See, e.g. U.S. Pat. No. 4,816,567 and Mage et al., in Monoclonal Antibody Production Techniques and Applications, pp. 79-97 (Marcel Dekker, Inc.: New York, 1987).

Thus, the modifier “monoclonal” indicates the character of the antibody as being obtained from a substantially homogeneous population of antibodies, and is not to be construed as requiring production of the antibody by any particular method. For example, the monoclonal antibodies of the presently disclosed subject matter can be made by the hybridoma method first described by Kohler and Milstein, Nature, 256:495 (1975), or can be made by recombinant DNA methods such as described in U.S. Pat. No. 4,816,567. The “monoclonal antibodies” can also be isolated from phage libraries generated using the techniques described in McCafferty et al., Nature, 348:552-554 (1990), for example.

“Humanized” forms of non-human (e.g. murine) antibodies are specific chimeric immunoglobulins, immunoglobulin chains, or fragments thereof (such as Fv, Fab, Fab′, F(ab′)₂ or other antigen-binding subsequences of antibodies) which contain minimal sequence derived from non-human immunoglobulin. For the most part, humanized antibodies are human immunoglobulins (recipient antibody) in which residues, from a hypervariable region of the recipient are replaced by residues from a hypervariable region of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody can comprise residues which are found neither in the recipient antibody or the donor antibody. These modifications are made to further refine and optimize antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin.

The term “hypervariable region” when used herein refers to the amino acid residues of an antibody which are responsible for antigen-binding. The hypervariable region comprises amino acid residues from a “complementarity determining region” or “CDR” (i.e. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework” or “FR” residues are those variable domain residues other than the hypervariable region residues as herein defined.

VI.A. Purification and Modification of Monoclonal Antibodies

Monoclonal antibodies produced in accordance with the presently disclosed subject matter can optionally be further purified. As would be appreciated by one of ordinary skill in the art, purification techniques can comprise filtration, centrifugation and various chromatographic methods such as HPLC or affinity chromatography. Techniques for purification of monoclonal antibodies, including techniques for producing pharmaceutical-grade antibodies, are well known in the art.

In some embodiments, antibodies of the presently disclosed subject matter can be provided in purified or substantially purified form. By way of example and not limitation, an antibody can be present in a composition that is substantially free of other polypeptides, e.g. where less than about 99%, 98%, 97%, 96%, 95%, 90%, 85%, 80%, 75%, 70%, 60%, or 50% (by weight) of the composition is made up of other polypeptides, or other contaminants.

In some embodiments, antibodies of the presently disclosed subject matter can be coupled to a drug for delivery to a treatment site or coupled to a detectable label to facilitate imaging of a site comprising cells of interest, such as cancer cells. Methods for coupling antibodies to drugs and detectable labels are well known in the art, as are methods for imaging using detectable labels.

In some embodiments, antibodies of the presently disclosed subject matter can be attached to a solid support, matrix or surface. As would be appreciated by one of ordinary skill in the art, attachment of an antibody of the presently disclosed subject matter to a solid support, matrix or surface can facilitate the use of the antibody in screening assays, diagnostic tests, and investigative arrays.

VI.B. Antibody Variants

The term “isolated”, as used in the context of a nucleic acid or polypeptide, indicates that the nucleic acid or polypeptide exists apart from its native environment and is not a product of nature. An isolated nucleic acid or polypeptide can exist in a purified form or can exist in a non-native environment such as a transgenic host cell.

The term “conservatively substituted variant” refers to an antibody comprising an amino acid residue sequence substantially identical to a sequence of a reference ligand of a target in which one or more residues have been conservatively substituted with a functionally similar residue and which displays the targeting activity as described herein. The phrase “conservatively substituted variant” also includes antibodies wherein a residue is replaced with a chemically derivatized residue, provided that the resulting peptide displays targeting activity as disclosed herein.

Examples of conservative substitutions include the substitution of one non-polar (hydrophobic) residue such as isoleucine, valine, leucine or methionine for another; the substitution of one polar (hydrophilic) residue for another such as between arginine and lysine, between glutamine and asparagine, between glycine and serine; the substitution of one basic residue such as lysine, arginine or histidine for another; or the substitution of one acidic residue, such as aspartic acid or glutamic acid for another.

Antibodies of the presently disclosed subject matter also include amino acid sequences comprising one or more additions and/or deletions or residues relative to that of a monoclonal antibody produced in accordance with the disclosed methods, so long as the requisite targeting activity of the peptide is maintained. The term “fragment” refers to an amino acid residue sequence shorter than that of a sequence of the presently disclosed subject matter, or of a wild-type or full-length sequence.

In some embodiments, the derivatives, fragments and variants of a antibody produced in accordance with the disclosed methods have the same or substantially the same immunogenic properties as the antibody from which they are derived. For example, a derivative, fragment or variant of a given monoclonal antibody can have substantially the same binding activity to an antigen as the monoclonal antibody itself. In some embodiments, derivatives, fragments or variants of a given monoclonal antibody can be equally as useful, or have substantially equivalent utility to a monoclonal antibody, as antibodies against a desired antigen, or for use in therapeutic compositions, diagnostic compositions, and combinations thereof.

Fragments, variants or derivatives of the monoclonal antibodies of the presently disclosed subject matter can be tested for their immunogenicity and/or binding activity using standard assays know to those of ordinary skill in the art. For example, competitive binding assays can be used to compare the immunogenicity of an antibody fragment with one or more monoclonal antibodies produced in accordance with the presently disclosed subject matter. A competitive binding assay can rely on the ability of a labeled standard antibody to compete with a test antibody fragment for binding with a limited amount of antigen. In some embodiments, sandwich-based assays can be used to determine the immunogenicity of an antibody fragment, variant or derivative. Sandwich assays involve the use of two antibodies, each capable of binding to a different immunogenic portion, or epitope, of the protein to be detected. In a sandwich assay, the test sample analyte is bound by a first antibody which is immobilized on a solid support, and thereafter a second antibody binds to the analyte, thus forming an insoluble three-part complex. See, e.g., U.S. Pat. No. 4,376,110. The second antibody can itself be labeled with a detectable moiety (direct sandwich assays) or can be measured using an anti-immunoglobulin antibody that is labeled with a detectable moiety (indirect sandwich assay). For example, one type of sandwich assay is an ELISA assay, in which case the detectable moiety is an enzyme.

Additional residues can also be added at either terminus for the purpose of providing a “linker” by which the monoclonal antibodies of the presently disclosed subject matter can be conveniently affixed to a label or solid matrix, or carrier. Amino acid residue linkers are usually at least one residue and can be 40 or more residues, more often 1 to 10 residues. Typical amino acid residues used for linking are tyrosine, cysteine, lysine, glutamic and aspartic acid, or the like. In addition, a peptide can be modified by terminal-NH₂ acylation (e.g., acetylation, or thioglycolic acid amidation) or by terminal-carboxylamidation (e.g., with ammonia, methylamine, and the like terminal modifications). Terminal modifications are useful, as is well known, to reduce susceptibility by proteinase digestion, and therefore serve to prolong the half life of the antibodies in solutions, particularly biological fluids where proteases can be present.

Nucleic Acids Encoding Antibodies. The terms “nucleic acid molecule” or “nucleic acid” each refer to deoxyribonucleotides or ribonucleotides and polymers thereof in single-stranded or double-stranded form. Unless specifically limited, the term encompasses nucleic acids containing known analogues of natural nucleotides that have similar properties as the reference natural nucleic acid. The terms “nucleic acid molecule” or “nucleic acid” can also be used in place of “gene”, “cDNA”, or “mRNA”. Nucleic acids can be synthesized, or can be derived from any biological source, including any organism.

The term “substantially identical”, as used herein to describe a degree of similarity between nucleotide sequences, refers to two or more sequences that have in some embodiments at least about 60%, in some embodiments at least about 65%, in some embodiments at least about 70%, in some embodiments at least about 75%, in some embodiments at least about 80%, in some embodiments at least about 85%, in some embodiments at least about 90%, in some embodiments at least about 93%, in some embodiments at least about 95%, in some embodiments at least about 96%, in some embodiments at least about 97%, in some embodiments at least about 98%, and in some embodiments at least about 99% nucleotide identity, as measured using one of the following sequence comparison algorithms (described hereinbelow) or by visual inspection. The substantial identity exists in nucleotide sequences of in some embodiments at least about 100 residues, in some embodiments at least about 150 residues, and in some embodiments in nucleotide sequences comprising a full length coding sequence.

Thus, substantially identical sequences can comprise mutagenized sequences, including sequences comprising silent mutations, or variably synthesized sequences. A mutation or variant sequence can comprise a single base change.

Another indication that two nucleotide sequences are substantially identical is that the two molecules specifically or substantially hybridize to each other under stringent conditions. In the context of nucleic acid hybridization, two nucleic acid sequences being compared can be designated a “probe” and a “target”. A “probe” is a reference nucleic acid molecule, and a “target” is a test nucleic acid molecule, often found within a heterogeneous population of nucleic acid molecules. A “target sequence” is synonymous with a “test sequence”.

An exemplary nucleotide sequence that can be employed for hybridization studies or assays includes probe sequences that are complementary to or mimic at least an about 14 to 40 nucleotide sequence of a nucleic acid molecule of the presently disclosed subject matter. For this purpose, a probe comprises a region of the nucleic acid molecule other than a sequence encoding a common immunoglobulin region. Thus, a probe comprises in some embodiments a sequence encoding a domain of the antibody that comprises an antigen-binding site. In some embodiments, probes comprise 14 to 20 nucleotides, or even longer where desired, such as 30, 40, 50, 60, 100, 200, 300 nucleotides or up to the full length of a region that encodes an antigen binding site. Such fragments can be readily prepared by, for example, chemical synthesis of the fragment, by application of nucleic acid amplification technology, or by introducing selected sequences into recombinant vectors for recombinant production.

The phrase “hybridizing specifically to” refers to the binding, duplexing, or hybridizing of a molecule only to a particular nucleotide sequence under stringent conditions when that sequence is present in a complex nucleic acid mixture (e.g., total cellular DNA or RNA).

The phrase “hybridizing substantially to” refers to complementary hybridization between a probe nucleic acid molecule and a target nucleic acid molecule and embraces minor mismatches that can be accommodated by reducing the stringency of the hybridization media to achieve the desired hybridization.

“Stringent hybridization conditions” and “stringent hybridization wash conditions” in the context of nucleic acid hybridization experiments such as Southern and Northern blot analysis are both sequence- and environment-dependent. Longer sequences hybridize specifically at higher temperatures. An extensive guide to the hybridization of nucleic acids is found in Tijssen (1993) Laboratory Techniques in Biochemistry and Molecular Biology—Hybridization with Nucleic Acid Probes. Elsevier, New York. Generally, highly stringent hybridization and wash conditions are selected to be about 5° C. lower than thermal melting point (Tₘ) for the specific sequence at a defined ionic strength and pH. Typically, under “stringent conditions” a probe will hybridize specifically to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.1×SSC at 65° C. An example of stringent wash conditions is 15 minutes in 0.2×SSC buffer at 65° C. See Sambrook & Russell (2001) Molecular Cloning: a Laboratory Manual, 3rd ed. Cold Spring Harbor Laboratory Press, Cold Spring Harbor, N.Y., for a description of SSC buffer.

Often, a high stringency wash is preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides, is 15 minutes in 4× to 6×SSC at 40° C. For short probes (e.g., about 10 to 50 nucleotides), stringent conditions typically involve salt concentrations of less than about 1M Na⁺ ion, typically about 0.01 to 1M Na⁺ ion concentration (or other salts) at pH 7.0-8.3, and the temperature is typically at least about 30° C. Stringent conditions can also be achieved with the addition of destabilizing agents such as formamide. In general, a signal to noise ratio of 2-fold (or higher) than that observed for an unrelated probe in the particular hybridization assay indicates detection of a specific hybridization.

The following are examples of hybridization and wash conditions that can be used to identify nucleotide sequences that are substantially identical to reference nucleotide sequences of the presently disclosed subject matter: in some embodiments a probe nucleotide sequence hybridizes to a target nucleotide sequence in 7% sodium dodecyl sulfate (SDS), 0.5 M NaPO₄, 1 mM EDTA at 50° C. followed by washing in 2×SSC, 0.1% SDS at 50° C.; in some embodiments a probe and target sequence hybridize in 7% sodium dodecyl sulfate (SDS), 0.5 M NaPO₄, 1 mM EDTA at 50° C. followed by washing in 1×SSC, 0.1% SDS at 50° C.; in some embodiments a probe and target sequence hybridize in 7% sodium dodecyl sulfate (SDS), 0.5 M NaPO₄, 1 mM EDTA at 50° C. followed by washing in 0.5×SSC, 0.1% SDS at 50° C.; in some embodiments a probe and target sequence hybridize in 7% sodium dodecyl sulfate (SDS), 0.5 M NaPO₄, 1 mM EDTA at 50° C. followed by washing in 0.1×SSC, 0.1% SDS at 50° C.; and in some embodiments a probe and target sequence hybridize in 7% sodium dodecyl sulfate (SDS), 0.5 M NaPO₄, 1 mM EDTA at 50° C. followed by washing in 0.1×SSC, 0.1% SDS at 65° C.

A further indication that two nucleic acid sequences are substantially identical is that proteins encoded by the nucleic acids are substantially identical, share an overall three-dimensional structure, or are biologically functional equivalents. These terms are defined further hereinbelow. Nucleic acid molecules that do not hybridize to each other under stringent conditions are still substantially identical if the corresponding proteins are substantially identical. This can occur, for example, when two nucleotide sequences are significantly degenerate as permitted by the genetic code.

The term “conservatively substituted variants” refers to nucleic acid sequences having degenerate codon substitutions wherein the third position of one or more selected (or all) codons is substituted with mixed-base and/or deoxyinosine residues. See Batzer et al. (1991) Nucleic Acids Res 19:5081; Ohtsuka et al. (1985) J Biol Chem 260:2605-2608; Rossolini et al. (1994) Mol Cell Probes 8:91-98994.

The term “subsequence” refers to a sequence of nucleic acids that comprises a part of a longer nucleic acid sequence. An exemplary subsequence is a probe, described hereinabove, or a primer. The term “primer” as used herein refers to a contiguous sequence comprising in some embodiments about 8 or more deoxyribonucleotides or ribonucleotides, in some embodiments about 10-20 nucleotides, and in some embodiments about 20-30 nucleotides of a selected nucleic acid molecule. The primers of the presently disclosed subject matter encompass oligonucleotides of sufficient length and appropriate sequence so as to provide initiation of polymerization on a nucleic acid molecule of the presently disclosed subject matter.

The term “elongated sequence” refers to an addition of nucleotides (or other analogous molecules) incorporated into the nucleic acid. For example, a polymerase (e.g., a DNA polymerase) can add sequences at the 3′ terminus of the nucleic acid molecule. In addition, the nucleotide sequence can be combined with other DNA sequences, such as promoters, promoter regions, enhancers, polyadenylation signals, intronic sequences, additional restriction enzyme sites, multiple cloning sites, and other coding segments.

Nucleic acids of the presently disclosed subject matter can be cloned, synthesized, recombinantly altered, mutagenized, or combinations thereof. Standard recombinant DNA and molecular cloning techniques used to isolate nucleic acids are known in the art. Site-specific mutagenesis to create base pair changes, deletions, or small insertions are also known in the art. See e.g., Sambrook & Russell (2001) Molecular Cloning: a Laboratory Manual, 3rd ed. Cold Spring Harbor Laboratory Press, Cold Spring Harbor, N.Y.; Silhavy et al. (1984) Experiments with Gene Fusions. Cold Spring Harbor Laboratory, Cold Spring Harbor, N.Y.; Glover & Hames (1995) DNA Cloning: A Practical Approach, 2nd ed. IRL Press at Oxford University Press, Oxford/New York; Ausubel (1995) Short Protocols in Molecular Biology, 3rd ed. Wiley, New York.

TABLE 1
Functionally Equivalent Codons

| | |
|---|---|
| Amino Acids | Codons |

| | | | | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic Acid | Asp | D | GAC | GAU | | | | |
| Glumatic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | ACG | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

It will also be understood by those of skill in the art that amino acid and nucleic acid sequences can include additional residues, such as additional N- or C-terminal amino acids or 5′ or 3′ nucleic acid sequences, and yet still be essentially as set forth in one of the sequences disclosed herein. The addition of terminal sequences particularly applies to nucleic acid sequences which can, for example, include various non-coding sequences flanking either of the 5′ or 3′ portions of the coding region or can include various internal sequences, i.e., introns, which are known to occur within genes.

Antibody Polypeptides. The term “substantially identical”, as used herein to describe a level of similarity between polypeptides comprising an antibody targeting ligand refers to a sequence having in some embodiments at least about 45%, in some embodiments at least about 50%, in some embodiments at least about 60%, in some embodiments at least about 70%, in some embodiments at least about 80%, in some embodiments at least about 90%, in some embodiments at least about 95%, in some embodiments at least about 96%, in some embodiments at least about 97%, in some embodiments at least about 98%, and in some embodiments at least about 99% sequence identity to a given sequence, when compared over the full length of the polypeptide. The term “full length”, as used herein to describe an antibody comprising an amino acid sequence that has not been truncated, shortened or interrupted, and has a number of amino acids as would be found in the antibody as naturally produced. Methods for determining percent identity are defined herein.

Substantially identical polypeptides can also encompass two or more polypeptides sharing a conserved three-dimensional structure. Computational methods can be used to compare structural representations, and structural models can be generated and easily tuned to identify similarities around important active sites or ligand binding sites. See Saqi et al. (1999) Bioinformatics 15:521-522; Barton (1998) Acta Crystallogr D Biol Crystallogr 54:1139-1146; Henikoff et al. (2000) Electrophoresis 21:1700-1706; Huang et al. (2000) Pac Symp Biocomput 5:227-238.

Substantially identical proteins also include proteins comprising an amino acid sequence comprising amino acids that are functionally equivalent to amino acids of a given sequence. The term “functionally equivalent” in the context of amino acid sequences is known in the art and is based on the relative similarity of the amino acid side-chain substituents. Henikoff & Henikoff (1992) Proc Natl Acad Sci USA 89:10915-10919; Henikoff et al. (2000) Electrophoresis 21:1700-1706. Relevant factors for consideration include side-chain hydrophobicity, hydrophilicity, charge, and size. For example, arginine, lysine, and histidine are all positively charged residues; that alanine, glycine, and serine are all of similar size; and that phenylalanine, tryptophan, and tyrosine all have a generally similar shape. By this analysis, described further hereinbelow, arginine, lysine, and histidine; alanine, glycine, and serine; and phenylalanine, tryptophan, and tyrosine; are defined herein as biologically functional equivalents.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte & Doolittle (1982) J Mol Biol 157:105-132). It is known that certain amino acids can be substituted for other amino acids having a similar hydropathic index or score and still retain a similar biological activity, for example binding activity. In making changes based upon the hydropathic index, amino acids can be substituted whose hydropathic indices are in some embodiments within ±2 of the original value, in some embodiments within ±1 of the original value, and in some embodiments within ±0.5 of the original value.

It is also understood in the art that the substitution of like amino acids can be made effectively on the basis of hydrophilicity. U.S. Pat. No. 4,554,101 describes that the greatest local average hydrophilicity of a protein, as governed by the hydrophilicity of its adjacent amino acids, correlates with its immunogenicity and antigenicity, e.g., with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, amino acids can be substituted whose hydrophilicity values are in some embodiments within ±2 of the original value, in some embodiments within ±1 of the original value, and in some embodiments within ±0.5 of the original value.

The term “substantially identical” also encompasses polypeptides that are biologically functional equivalents. The term “functional”, as used herein to describe antibody-based targeting ligands, refers two or more antibodies that are immunoreactive with a same target molecule. In some embodiments, the two or more antibodies specifically bind a same target molecule and substantially lack binding to a control antigen.

The term “specifically binds”, when used to describe binding of an antibody to a target molecule, refers to binding to a target molecule in a heterogeneous mixture of other polypeptides.

The phases “substantially lack binding” or “substantially no binding”, as used herein to describe binding of an antibody to a control polypeptide or sample, refers to a level of binding that encompasses non-specific or background binding, but does not include specific binding.

Techniques for detecting antibody-target molecule complexes are known in the art and include but are not limited to centrifugation, affinity chromatography, ELISA, immunoprecipitation, flow cytometry and other immunochemical methods as known to those of ordinary skill in the art and as disclosed herein.

The presently disclosed subject matter also provides functional fragments of an antibody polypeptide. Such functional portion need not comprise all or substantially all of the amino acid sequence of a monoclonal antibody of the presently disclosed subject matter.

The presently disclosed subject matter also includes functional polypeptide sequences that are longer sequences than that of a monoclonal antibody of the presently disclosed subject matter. For example, one or more amino acids can be added to the N-terminus or C-terminus of a monoclonal antibody of the presently disclosed subject matter. Methods of preparing such proteins are known in the art. In some embodiments, the monoclonal antibodies of the presently disclosed subject matter can be in the form of dimers and in some embodiments other multimeric formations. In some embodiments tumor accumulation of a small antibody is improved by increasing its molecular weight by dimerization. In addition to making homodimeric constructs, heterodimeric constructs comprising two different monoclonal antibodies can be constructed in some embodiments. In some embodiments, in order to confer conformational flexibility on the molecule, two domains can be connected by a linker, as discussed herein.

Isolated polypeptides and recombinantly produced polypeptides can be purified and characterized using a variety of standard techniques that are known to the skilled artisan. See e.g., Schröder & Lübke (1965) The Peptides, Academic Press, New York; Schneider & Eberle (1993) Peptides, 1992: Proceedings of the Twenty-Second European Peptide Symposium, Sep. 13-19, 1992, Interlaken, Switzerland, Escom, Leiden; Bodanszky (1993) Principles of Peptide Synthesis, 2nd rev. ed. Springer-Verlag, Berlin/New York; Ausubel (1995) Short Protocols in Molecular Biology, 3rd ed. Wiley, New York.

Nucleotide and Amino Acid Sequence Comparisons. The terms “identical” or percent “identity” in the context of two or more nucleotide or polypeptide sequences, refer to two or more sequences or subsequences that are the same or have a specified percentage of amino acid residues or nucleotides that are the same, when compared and aligned for maximum correspondence, as measured using one of the sequence comparison algorithms disclosed herein or by visual inspection.

The term “substantially identical” in regards to a nucleotide or polypeptide sequence means that a particular sequence varies from the sequence of a naturally occurring sequence, or a given sequence as disclosed herein, by one or more deletions, substitutions, or additions, the net effect of which is to retain biological activity of a gene, gene product, or sequence of interest.

For sequence comparison, typically one sequence acts as a reference sequence to which test sequences are compared. When using a sequence comparison algorithm, test and reference sequences are entered into a computer program, subsequence coordinates are designated if necessary, and sequence algorithm program parameters are selected. The sequence comparison algorithm then calculates the percent sequence identity for the designated test sequence(s) relative to the reference sequence, based on the selected program parameters.

Optimal alignment of sequences for comparison can be conducted, for example, by the local homology algorithm of Smith & Waterman (1981) Adv Appl Math 2:482-489, by the homology alignment algorithm of Needleman & Wunsch (1970) J Mol Biol 48:443-453, by the search for similarity method of Pearson & Lipman (1988) Proc Natl Acad Sci USA 85:2444-2448, by computerized implementations of these algorithms (e.g., programs available in the DISCOVERY STUDIO® package from Accelrys, Inc., San Diego, Calif., United States of America), or by visual inspection. See generally Ausubel (1995) Short Protocols in Molecular Biology, 3rd ed. Wiley, New York.

An exemplary algorithm for determining percent sequence identity and sequence similarity is the BLAST algorithm, which is described in Altschul et al. (1990) J Mol Biol 215:403-410. Software for performing BLAST analyses is publicly available through the website of the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength W=11, an expectation E=10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See Henikoff & Henikoff, 1992.

In addition to calculating percent sequence identity, the BLAST algorithm also performs a statistical analysis of the similarity between two sequences. See e.g., Karlin & Altschul (1993) Proc Natl Acad Sci USA 90:5873-5877. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in some embodiments less than about 0.1, in some embodiments less than about 0.01, and in some embodiments less than about 0.001.

VI.C. Antibody Derivatives

In some embodiments, the derivatives, fragments and variants of a antibody of the presently disclosed subject matter can have the same or substantially the same immunogenic properties as a antibody of the presently disclosed subject matter from which they are derived. For example, a derivative, fragment or variant of a monoclonal antibody of the presently disclosed subject matter can have substantially the same binding activity to a desired antigen, e.g. HIV, as the monoclonal antibody from which it was derived. In some embodiments, derivatives, fragments or variants of a given monoclonal antibody can be equally as useful, or have substantially equivalent utility to full-length monoclonal antibodies.

### VII. Recombinant Expression

In some embodiments of the presently disclosed subject matter, a monoclonal antibody produced or identified using the disclosed methods can be expressed recombinantly. In some embodiments, the presently disclosed subject matter provides a method for producing monoclonal antibodies, comprising the steps of: (i) providing to a primary B cell in culture an activator of B cell proliferation in the presence of an inhibitor of a host innate response to activator-mediated proliferative signals; (ii) screening the supernatant from the culture for antigen specificity; (iii) isolating from the culture a B cell capable of producing a monoclonal antibody having the desired antigen specificity; and (iv) expressing the monoclonal antibody, comprising (a) culturing the B cell under conditions where the monoclonal antibody is expressed; or (b) obtaining a nucleic acid encoding the antibody of interest from the isolated B cell; (c) employing the nucleic acid to prepare an expression host that can express the antibody of interest; and (d) culturing the expression host under conditions where the antibody of interest is expressed.

In some embodiments, limited dilution cloning of activated and/or immortalized B cells of the presently disclosed subject matter can provide a a EBV-tranformed cell line capable of producing monoclonal antibodies.

In some embodiments, electrofusion of activated and/or immortalized B cells of the presently disclosed subject matter with another cell line, i.e. a myeloma line, can be employed to create a hybridoma. Generation of a hybridoma can provide for long-term and stable antibody production. The hybridoma method is described by Kohler and Milstein, Nature, 256:495 (1975).

In some embodiments, B cells activated and/or transformed in accordance with the presently disclosed subject matter can be used as a source of polypeptide and/or polynucleotide sequences of a monoclonal antibody for use in subsequent recombinant expression. In some embodiments, the nucleotide sequence encoding an antibody of the presently disclosed subject matter can be sequenced and thereafter employed in a heterologous expression system, e.g. 293 cells or CHO cells. In some embodiments, an antibody can be recombinantly expressed by obtaining one or more nucleic acids (e.g. heavy and/or light chain genes) from the a B cell clone that encodes the antibody of interest and inserting the nucleic acid into an expression host in order to permit expression of the antibody of interest in that host.

In some embodiments, the polypeptide sequence of an antibody of the presently disclosed subject matter can be sequenced by methods known to those of skill in the art, e.g. Mass Spectroscopy, de novo sequencing. In some embodiments, the polypeptide sequence could then be used to engineer a nucleotide sequence encoding the antibody, which could be cloned into an expression vector and expressed in an expression host. In some embodiments the expression host is a prokaryotic or eukaryotic cell.

In some embodiments, recombinant expression of an antibody of the presently disclosed subject matter can provide for the expression of a fragment of an antibody. In particular, recombinant expression can provide for the expression of a scFv, Fab, Fab₂, Fab₃, V-NAR, V-domain, camelid Ab, diabody, tribody, tetrabody, minibody, or nanobody of a given antibody.

Production of antibodies using recombinant DNA methods is described in U.S. Pat. No. 4,816,567. For recombinant production of the antibody, the nucleic acid encoding it is isolated and inserted into a replicable vector for further cloning (amplification of the DNA) or for expression. DNA encoding the monoclonal antibody is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence. Examples of such expression system components are disclosed in U.S. Pat. No. 5,739,277.

Suitable host cells for cloning or expressing the DNA in the vectors herein are the prokaryote, yeast, or higher eukaryote cells (see, e.g., U.S. Pat. No. 5,739,277).

Host cells are transformed with the above-described expression or cloning vectors for antibody production and cultured in conventional nutrient media modified as appropriate for inducing promoters, selecting transformants, or amplifying the genes encoding the desired sequences. The host cells used to produce the antibody of the presently disclosed subject matter can be cultured in a variety of media. Any necessary supplements can also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium.

If the antibody is produced intracellularly, as a first step, the particulate debris, either host cells or lysed fragments, is removed, for example, by centrifugation or ultrafiltration. Where the antibody is secreted into the medium, supernatants from such expression systems are generally first concentrated using a commercially available protein concentration filter, for example; an Amicon™ or Millipore™ Pellicon™ ultrafiltration unit A protease inhibitor such as PMSF can be included in any of the foregoing steps to inhibit proteolysis and antibiotics can be included to prevent the growth of adventitious contaminants.

In the recombinant production of antibodies of the presently disclosed subject matter, the nucleic acid used in the expression host can be manipulated to insert, delete or amend certain nucleic acid sequences. Changes from such manipulation include, but are not limited to, changes to introduce restriction sites, to amend codon usage, to add or optimise transcription and/or translation regulatory sequences. It is also possible to change the nucleic acid to alter the encoded amino acids. For example, it can be useful to introduce one or more (e.g. 1, 2, 3, 4, 5, 6, 7, 8, 9, 10, etc.) amino acid substitutions, one or more (e.g. 1, 2, 3, 4, 5, 6, 7, 8, 9, 10, etc.) amino acid deletions and/or one or more (e.g 1, 2, 3, 4, 5, 6, 7, 8, 9, 10, etc.) amino acid insertions into the antibody's amino acid sequence. Such point mutations can modify effector functions, antigen-binding affinity, post-translational modifications and immunogenicity. Additionally, such manipulations can provide for the introduction of amino acids for the attachment of covalent groups (e.g. labels) or to act as tags (e.g. for purification purposes). Mutations can be introduced in specific sites or can be introduced at random, followed by selection (e.g. molecular evolution).

The antibody composition prepared from the cells can be purified using, for example, hydroxylapatite chromatography, gel electrophoresis, dialysis, and affinity chromatography, with affinity chromatography being the preferred purification technique.

### VIII. Kits Containing Antibodies

In a further embodiment of the presently disclosed subject matter, there are provided articles of manufacture and kits containing antibodies produced in accordance with the presently disclosed subject matter which can be used, for instance, for therapeutic or non-therapeutic applications described above. The article of manufacture comprises a container with a label. Suitable containers include, for example, bottles, vials, and test tubes. The containers can be formed from a variety of materials such as glass or plastic. The container holds a composition which includes an active agent that is effective for therapeutic or non-therapeutic applications, such as described above. The active agent in the composition can comprise the antibody. The label on the container indicates that the composition is used for a particular therapy or non-therapeutic application, and can also indicate directions for either in vivo or in vitro use, such as those described above.

In some embodiments, a kit can comprise compositions for use in transforming B cells, activating B cells, increasing B cell proliferation, or producing antibodies. In some embodiments the kit can comprise B cell activators and inhibitors of host innate response to activator-mediated proliferation signals.

The kit of the presently disclosed subject matter will typically comprise the container described above and one or more other containers comprising materials desirable from a commercial and user standpoint, including buffers, diluents, filters, needles, syringes, and package inserts with instructions for use.

### EXAMPLES

The following examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### Example 1

### Activation of the DNA Damage Response Following EBV Infection of Primary Human B Lymphocytes

The hypothesis that a robust innate tumor suppressor pathway, e.g. the DNA damage response (DDR) pathway, is activated early after EBV infection leading to the inhibition of long-term proliferation was tested. Immune fluorescence experiments were performed in an effort to identify hallmarks of the DDR in purified primary B cells. CD19+ B cells were purified to greater than 95% homogeneity from normal donor peripheral blood mononuclear cells (PBMC) using a negative B cell isolation kit (BD Biosciences). Primary B cells were infected at a multiplicity of infection (MOI) of approximately 5 using the prototypical transforming EBV strain B95-8. Cells were analyzed at 7 days post infection relative to uninfected and irradiated (5Gy, 1 h) B cells for three markers of double-stranded breaks: phospho-ATM Ser1981 (P-ATM) (Bakkenist and Kastan, 2003), 53BP1 (DiTullio et al., 2002), and γ-H2AX (Burma et al., 2001). After EBV infection an increase in P-ATM and γ-H2AX positive cells was observed and a redistribution of 53BP1 from dense nuclear aggregates to punctuate nuclear foci similar to irradiated cells. Thus, EBV infection induced hallmarks of the DNA damage response.

### Example 2

### Defining the Kinetics of the EBV-Induced DDR

Next the kinetics of the EBV-induced DNA damage response were defined. Cells were infected as above and the expression of the early viral nuclear protein EBNA-LP and γ-H2AX was assayed at 1, 5, 7, 10, and 14 days post infection. An increase in γ-H2AX positive cells was observed, specifically in EBNA-LP positive cells at 5 and 7 days post infection. However, between 10 and 14 days post infection as well as in freshly-derived EBV-transformed lymphoblastoid cell lines (LCLs), weak to no γ-H2AX staining was observed in EBNA-LP positive cells. The few γ-H2AX positive cells at late time points were weakly EBNA-LP positive or negative and presumably undergoing apoptosis (Solier and Pommier, 2009; Solier et al., 2009). To verify that γ-H2AX detection is not due to cellular receptor binding or viral DNA introduction into the host cell, B cells were infected with UV-inactivated B95-8 virus. No increase in γ-H2AX staining was observed over background in these infected cells. As a control for infection, induction of interferon-regulated genes was verified to be similar between B95-8 and UV-inactivated B95-8 virus. This strongly suggests that EBV gene expression is required to induce the DNA damage response rather than EBV DNA per se. Finally, as a positive control for DNA damage, primary B cells were exposed to ionizing radiation (IR). Cells harvested at 1 h post IR stained positive for γ-H2AX as expected. All samples were stained concurrently and images used for analysis were captured with identical exposure times. Therefore, it was concluded that EBV induced a DNA damage response comparable to 0.1-0.5 Gy of ionizing radiation.

### Example 3

### ATM and Chk2 Kinases Suppressed EBV-Mediated Proliferation and Transformation

To further elicudate the functional role of the DDR pathway, a small molecule compound was used to antagonize the core DDR checkpoint kinase ATM. One such small molecule, an ATM inhibitor (ATMi), is compound KU-55933 (Hickson et al., 2004). This compound has been well characterized with respect to potency and specificity in cell culture as well as in vivo (Ding et al., 2006). Here, the long-term outgrowth of EBV-transformed LCLs was robustly increased in the presence of the inhibitor (FIG. 3A). This effect was substantial as 2 μM ATMi increased EBV-mediated transformation efficiency by approximately 3-fold and 5 μM ATMi by 30-fold.

Likewise, the downstream ATM target, Chk2, was evaluated for its ability to inhibition EBV-mediated proliferation of B cells. A 2-arylbenzamidazole Chk2 inhibitor, Chk2i II (Arienti et al., 2005), was employed during transformation of B cells. This compound shows exquisite specificity over Chk1 and over 30 other serine/threonine kinases (Arienti et al., 2005). Similar to ATM inhibition, Chk2 inhibition (Chk21) led to a dramatic and dose-dependent increase in EBV transformation efficiency (FIG. 3B).

### Example 4

### Characterization of ATM and Chk2 Kinase Suppression of EBV-Mediated Proliferation and Transformation

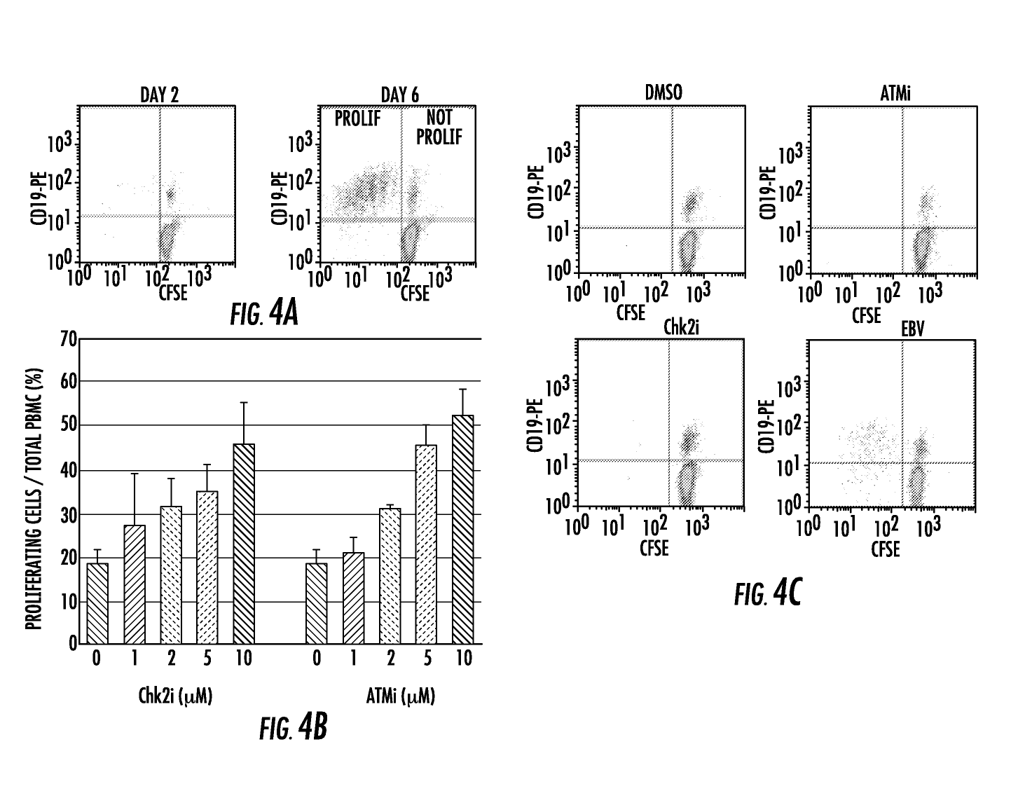

To further characterize the ATM- and Chk2-dependent block to EBV-mediated transformation, B cell proliferation early after infection was analyzed. PBMC were infected and B cell proliferation was followed over time using the fluorescent dye CFSE and CD19 staining of B cells. CFSE staining assays proliferation as its fluorescence is halved in daughter cells. PBMC were stained with the cell permeable dye carboxyfluorescein succinimidyl ester (CFSE). This dye enters the cytoplasm and is cleaved to fluoresce immediately by intracellular esterases. The dye is maintained in cells at a concentration that divides by half with each cell division. Thus, each two-fold reduction of CFSE fluorescence readout by flow cytometry correlates with one cycle of proliferation. CFSE-labeled PBMC were infected with EBV and B cell proliferation was monitored by CD19-PE or APC labeling cells at different days after infection. Flow cytometry was used to analyze the CD19 and CFSE levels in gated live cells (by forward versus side scatter). The percentage of CD19+ cells that have proliferated (CFSElo) relative to total PBMC cell numbers were plotted to indicate a relative proliferation index in panel B of FIG. 4. Using the CD19 marker and CFSE the proliferation of B cells during the early stages of transformation were specifically followed (FIG. 4A). Infection in the presence of either ATM or Chk2 inhibitor led to a dose dependent increase in B-cell proliferation (FIG. 4B). No effect of the ATM or Chk2 inhibitors on B cell proliferation was observed in the absence of EBV (FIG. 4C).

### Example 5

### Washout and Time of Addition Experiments Using Either ATM or Chk2 Inhibitor

The above experiments were followed with washout and time of addition experiments using either ATM or Chk2 inhibitor. EBV-infected B cells were determined to be sensitive to the inhibitors between 4 and 8 days after infection. For example, when either inhibitor was added within the first 4 days of infection the effect on proliferation at 14 days was as pronounced as if the inhibitor was added at day 0. However, if the inhibitor was added after day 8, there was no effect on proliferation at day 14. Conversely, if the inhibitor was washed out within 4 days after infection, then the increased proliferation at 14 days was not observed. Similar results were obtained in long-term transformation assays (data not shown). Therefore, during a critical period of time (4-8 days) following infection, EBV-induced proliferation and long-term outgrowth is suppressed by ATM and Chk2. These data collectively establish that an activated DNA damage response suppresses EBV-induced proliferation early after infection.

### Example 6

### EBV Induced Hyper-Proliferation Early after Infection

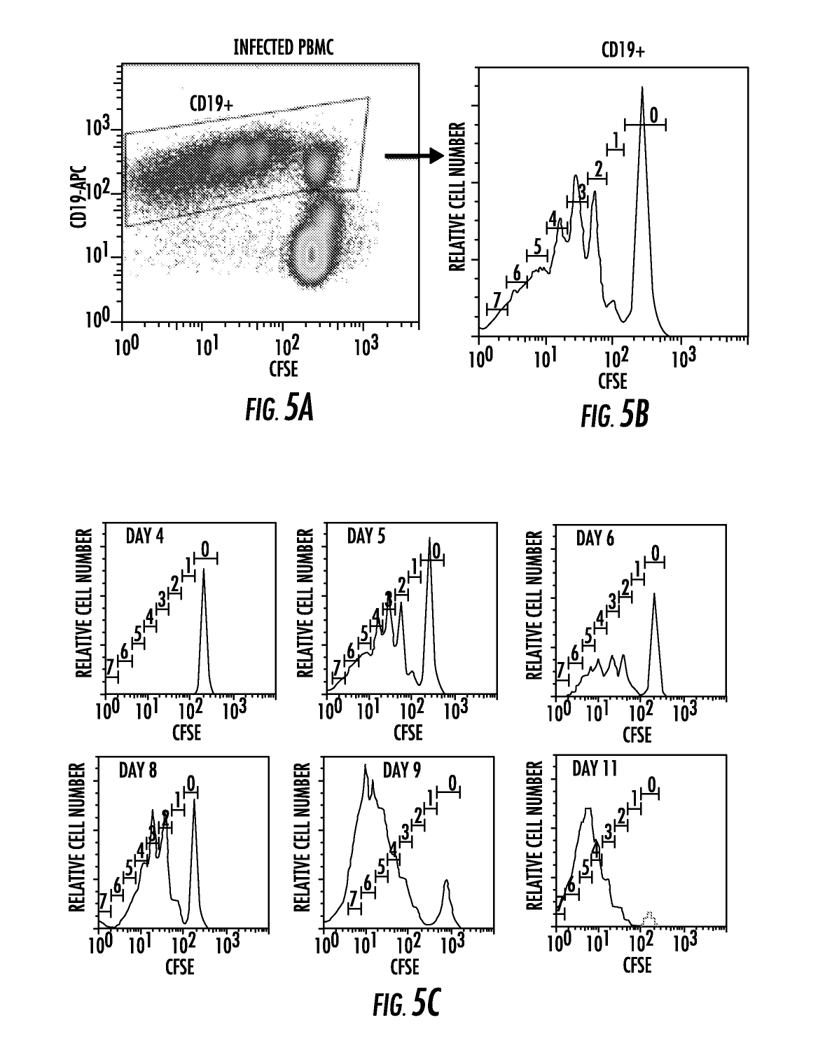

Experiments were conducted in an effort to understand the changes occurring during this period 4 to 8 days after infection that might contribute to DDR sensitivity. In performing the CFSE experiments proliferating cells were consistently observed 5 days after infection, but little to none were observed prior to that. When proliferating cells appeared between day 4 and day 5, it was noted that there were always cells that had divided more than once by CFSE staining. CFSE profiles of CD19+ B cells (example shown in 5A) at different days following infection are shown in FIG. 5. In FIG. 5C it is apparent that between day 4 and day 5 a burst of proliferation occurred with some cells having doubled three or four times (i.e. having 8 to 16-fold less CFSE than non-proliferating). At later days post infection, cells appeared to proliferate at a slower rate.

### Example 7

### Characterization of EBV Induced Hyper-Proliferation

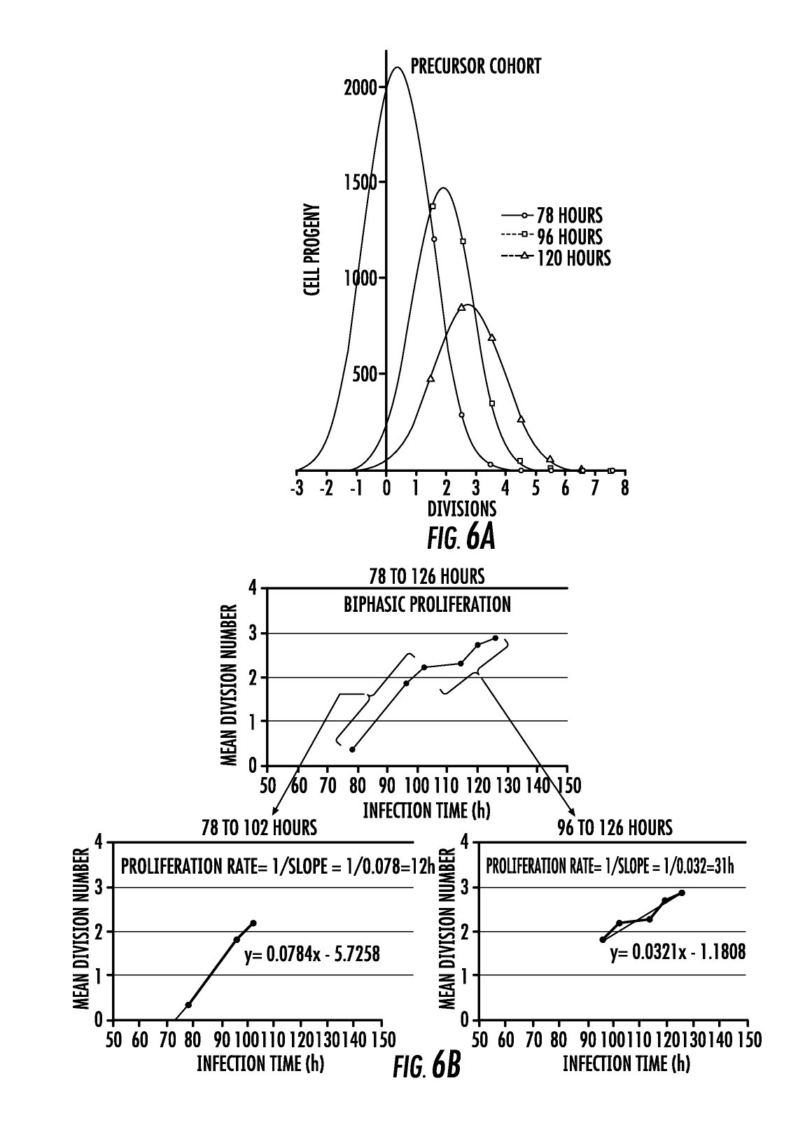

Experiments were conducted to characterize the early kinetics of EBV-induced proliferation. CFSE labeled PBMC were infected and cells were analyzed at time points prior to and during the first several rounds of proliferation. The CFSE profiles of CD19+ B cells were used to determine the proliferation rate of cells at different times after infection (Hawkins et al., 2007). The mean division index at each time point was determined by fitting the number of cells relative its precursor cohort (i.e. normalizing based on the fact that each doubling gives rise to two cells) to a Gaussian distribution (FIG. 6A). The proliferation rate was then determined by plotting the mean division number as a function of time. The slope of this function directly correlates with the proliferation rate (Hawkins et al., 2007). Consistent with FIG. 5, EBV induced a bi-phasic proliferation where the initial cycles were faster than later cycles (FIG. 6B). Separating these two curves gave approximate proliferation rates of 12 h for the early cycles and 31 h for the later cycles (FIG. 6B, lower), the latter similar to the ˜24 h rate of normal LCL cell cycles. These data implicate a previously unrecognized difference between cells initiating proliferation after EBV infection with those after several rounds of division. Given these preliminary findings the next step was to determine whether proliferation rate and capacity were linked to the number of times a cell had previously divided.

### Example 8

### The Proliferative Capacity of EBV-Infected Cells Early after Infection

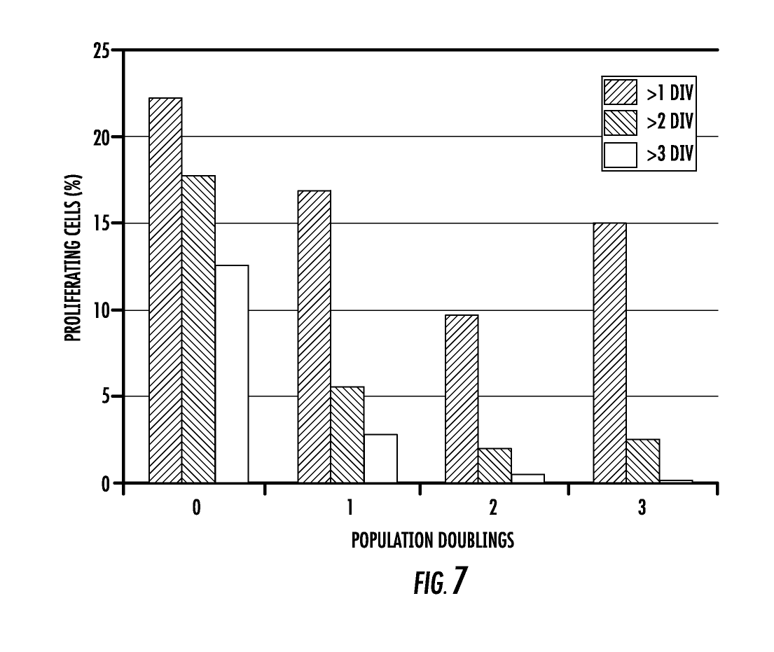

Inhibition of ATM or Chk2 between 4 and 8 days post infection increased EBV-mediated B cell proliferation and during this time period the majority of infected B cells that will ultimately proliferate, begin to proliferate. Day 4 is when cells begin to proliferate and, by day 8, cells that have not begun to proliferate (population doubling 0, or PD0) will never proliferate. Moreover, the cells in PD0 that began proliferating between days 4 and 5 displayed an apparent ‘burst’ of proliferation (FIG. 6). Therefore, experiments were designed to determine whether cells sorted by CFSE 5 days post infection displayed different proliferative capacities upon returning them to culture for 24 h. CFSE profiles of cells sorted from PD0, 1, 2, and 3 (FIG. 7) were analyzed. The CFSE profiles of PD0 cells that had proliferated were overlayed with those of PD1, 2, and 3. These data clearly demonstrate that PD0 cells have the ability to divide more rapidly within 24 h than cells derived from PD1, 2, or 3. As expected, proliferative capacity was attenuated from PD0 to PD3 as demonstrated by plotting the percentage of sorted cells that divided more than 1, 2, or 3 times per 24 h (FIG. 7). Despite measuring an average initial proliferation rate of 12 h (FIG. 6B), CFSE-sorted PDs contain cells at different point in the cell cycle. Thus, cells that are in late mitosis within PD0 will proliferate to a later PD than those in G1. This concept explains the detection of cells dividing greater than twice within 24 h in FIG. 7.

### Example 9

### DDR Activation in Cells Sorted by Population Doubling

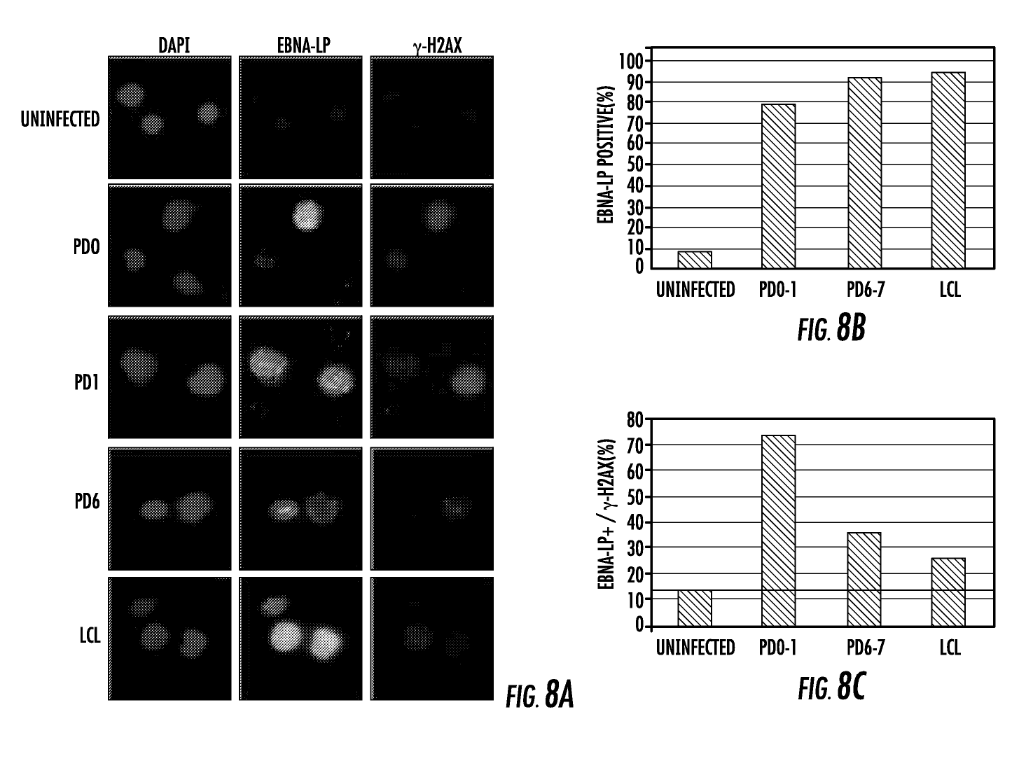

Experiments were designed to determine whether the hyper-proliferative phase early after infection induced the DNA damage response detected during a similar time period after infection. Rather than infecting cells and performing IF at different days after infection, cells were sorted at 5 and 8 days post infection based on population doubling and stained them for EBNA-LP and γ-H2AX. Experiments were designed to analyze cells that: a) were uninfected, b) had not yet divided or divided once (PD0-1), c) divided 6 or 7 times (PD6-7), and d) LCLs (FIG. 8). A robust increase in EBNA-LP positive cells that were also positive for γ-H2AX during PD0-2 were observed and this effect was attenuated in the PD6-7 cells and LCLs. Representative images captured the transient high level γ-H2AX-positively that is attenuated to a lower level in late proliferating PDs and LCLs though still above uninfected cell background (FIG. 8A). Using the Nikon Basic Research software package, 50-200 nuclei at each time point were analyzed and the EBNA-LP and γ-H2AX levels per cell were quantified. A threshold of 1.5× above background staining for positively was set and the percentage of i) EBNA-LP positive cells and ii) EBNA-LP positive/γ-H2AX positive cells in each population was calculated (FIG. 8B). These data quantitatively demonstrate that early proliferating cells have an increased level of γ-H2AX relative to late proliferating cells and LCLs. Moreover, these data strongly support the notion that the EBV-induced DNA damage response is correlated with an early period of hyper-proliferation.

### Summary of Examples 1-9

Examples 1-9: 1) identified an activated DNA damage response early after EBV infection, 2) provided evidence for the role of ATM and Chk2 as DNA damage effectors in suppressing EBV-mediated proliferation and transformation, and 3) defined a phase after infection characterized by a high proliferation rate during which the DNA damage response is activated and inhibitors of this response must be present to increase transformation efficiency.

### Example 10

### Inhibition of the DDR Increased EBV Transformation Efficiency of B Cells

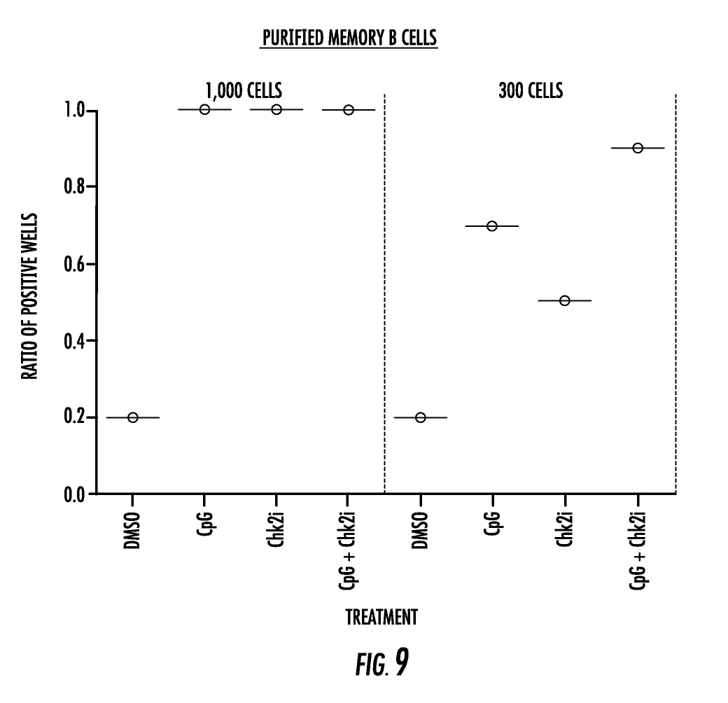

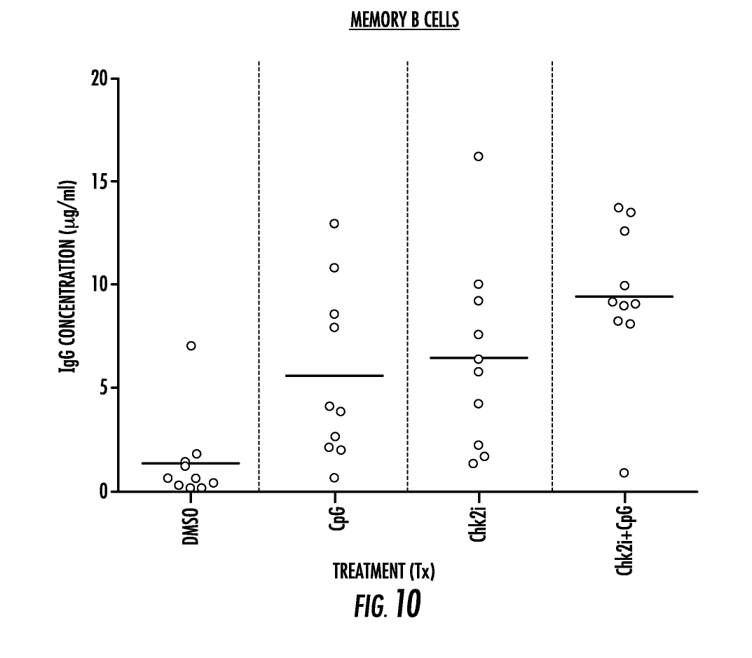

This Example pertains to the evaluation of whether inhibition of the DDR, in particular Chk2, is capable of increasing the efficiency of EBV transformation of memory B cells and whether the frequency of antibody secreting cells will increase. Data demonstrating this is shown in FIGS. 9 and 10. As a positive control, CpG 2006 TLR9 agonist was included either alone or together with Chk21. The CpG employed was CpG 2006 (Hartmann and Krieg, The Journal of Immunology, 2000, 164: 944-952.). The Chk2i employed was Chk2i II (Arienti et al., 2005 J. Med Chem 48:1873-1875).

Normal donor peripheral blood mononuclear cells (PBMC) were sorted to >95% pure IgG+ memory B cells using a scheme of positive selection for CD19 and CD27 and negative selection of IgM, IgD, and IgA. Cells were then infected with EBV B95-8 strain at an MOI of 5 and either DMSO, CpG (2.5 μg/mL), Chk2i (5 μM), or CpG +Chk21. Cells were seeded in 96-well plates with 50,000 lethally irradiated J774A.1 cells at different amounts. FIG. 9 shows 1,000 and 300 memory B cells per well. Wells were analyzed at 4 weeks for outgrowth and positive wells are plotted as a percentage for each condition. Both CpG and Chk2i increased the ability of B cells to grow out and together had an additive effect on outgrowth. FIG. 10 shows the Ig levels in the supernatants of each condition at 300 cells/well. These were measured by IgG-specific ELISA.

### Example 11

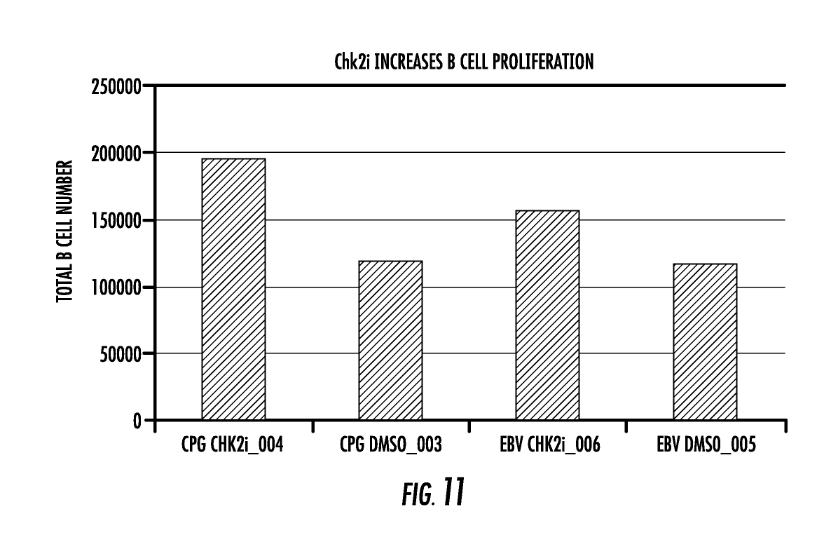

Inhibition of the DDR increased B Cell proliferation in response to mitogens This Example pertains to inhibition of the DDR signaling pathway as a means to increase B cell mitogen induced proliferation. In FIG. 11 inhibition of Chk2 increased the number of B cells proliferating following either CpG DNA treatment or EBV infection as B cell mitogens. These data were obtained by counting cell numbers using flow cytometry at 7 days following stimulation. Total B cell number refers to CD19+ cells gated for viability using forward and side scatter and normalized to polystyrene beads.

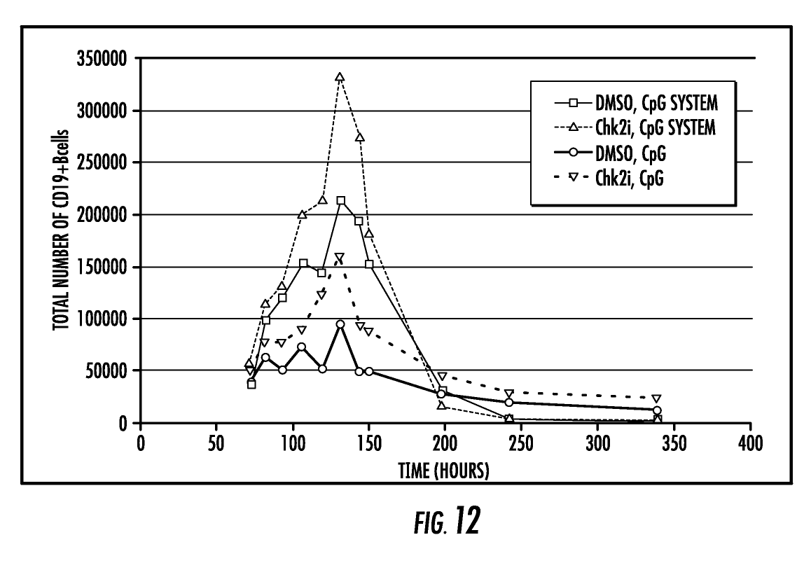

The proliferation of cells in response to a combination of mitogenic signals was also assayed in the presence of a DNA damage response inhibitor. FIG. 12 plots the number of B cells following stimulation with CpG or CpG system in the presence or absence of 5 μM Chk2i II. Cells were counted at different time points using the assay described in Example 10.

The CpG employed was CpG 2006 (Hartmann and Krieg, The Journal of Immunology, 2000, 164: 944-952.). The Chk2i employed was Chk2i II (Arienti et al., 2005 J. Med Chem 48:1873-1875). The CpG system mixture employed in this example is derived from that described by Kvell et al. (Kvell et al., Molecular Therapy Vol. 12, No. 5:892-899, November 2005). The mixture includes: CpG DNA system; CpG DNA (2.5 μg/ml), anti-Ig antibody (2 μg/ml), IL-2 (50 ng/ml), and IL-10 (10 ng/ml). The EBV infection was using a multiplicity of infection of 5 with the prototypical transforming strain B95-8.

### Example 12

### Identifying Additional Targets for Inhibition in a Host Innate Response to Activator-Mediated Proliferative Signals

This Example provides approaches for identifying additional targets for inhibition in a host innate response to activator-mediated proliferative signals For instance, the DDR, including ATM, recognizes double-stranded breaks in response to a wide range of stimuli including the genotoxic stress of ionizing radiation, the processing of collapsed replication forks, and exposed double-stranded DNA from viral genomes or telomeres. In each of these settings, the complex of proteins associated with ATM, the cell type and phase in the cell cycle, and the molecular nature of the nucleic acid signal all play a role in determining the substrates of ATM and ultimately the transcriptional program deciding the fate of the cell. Many direct substrates of ATM have been characterized, most notably by high-throughput proteomic analysis of irradiated 293T cells. Matsuoka, S., et al. (2007). Science 316, 1160-1166. Furthermore, the downstream ATM-dependent transcriptional program has been characterized in response to irradiation. Elkon, R., et al., (2005). Genome Biol 6, R43. However, no direct comparison has been performed between oncogenic stress-induced and genotoxic stress-induced ATM-dependent transcriptional targets in the same cell. This can play a role in understanding particular effectors of the ATM pathway in response to EBV-driven oncogenic stress.

Experiments have been performed analyzing the transcriptional changes in EBV-infected, early proliferating cells and monoclonal LCLs versus resting B cells from the same donor. Gene expression profiles that are coordinately up-regulated throughout transformation as well as those that distinguish the early proliferating population from B cells and LCLs were analyzed. This set of genes included those that were highly induced early after infection during initial proliferation and attenuated in expression during LCL outgrowth. This EBV-induced oncogenic stress signature was enriched for cell cycle regulatory genes (including CCNE2, CCNB1, MCM4, MCM7, CENPF, CDC20) and effectors of the DNA damage response (including FANCA, POLQ, PCNA, MSH5, EXO1, BRCA1, APEX1). These expression data provide additional targets for inhibition in a host innate response to activator-mediated proliferative signals.

Unsupervised hierarchical clustering of Human Exon Array 1.0ST was performed on data from 4 sets of uninfected B cells, EBV-infected early-proliferating cells, and their monoclonal LCL outgrowth. Some genes (e.g., CD226, NFKB2, ICAM1) were under-expressed initially then up-regulated while others (e.g., FOS, FCRL4) were over-expressed early and then attenuated. These data indicate that all three conditions self-segregate and can be uniquely identified. In addition, genes in common to only two sets clustered together as well. A set of genes that is differentially regulated in early-proliferating cells relative to LCLs can be identified.

RNA samples from six naïve B cell preparations and six Burkitt lymphoma cell lines were used to define an aberrant Myc B cell signature. Basso, K., et al. (2005) Nat Genet. 37, 382-390. This and other signatures defined in B cells are used to characterize the samples noted above. Also, the relative contribution of various oncogenic and tumor suppressive signatures (Bild, A. H., et al. (2006a). Nat Rev Cancer 6, 735-741; Bild, A. H., et al. (2006b). Nature 439, 353-357) to the LCL molecular phenotype was using RNA from four normal donor B cell preps and four monoclonal LCLs.

Additional experiments compare EBV-induced DNA damage to irradiation-induced damage in resting B cells and LCLs towards defining an ATM-dependent oncogenic stress signature. Expression is analyzed using AFFYMETRIX™ Human Exon Arrays (1.0ST), which provides a comprehensive signal of gene expression with probes across every exon of every human gene. Moreover, these arrays provide information about alternative exon usage that could be relevant for these samples. The groups of samples that are analyzed are set forth in Table 2.

TABLE 2

| | Condition A | Condition B | Condition C |
|---|---|---|---|
| Parameter | (basal) | (activated) | (inhibited) |
| 1. EBV- | Resting CD19 | EBV-infected, | Condition 1B + 5 |
| induced | positive B cells | early proliferating | μM ATMi |
| DNA damage | | cells (PD1-3) | |
| 2. Genotoxic | Resting CD19 | B cells → 1 Gy | Condition 2B + 5 |
| stress | positive B cells | irradiation (IR) | μM ATMi |
| 3. Proliferating | Monoclonal LCL | Monoclonal LCL | Condition 3B + 5 |
| | from 1B infection | → 1 Gy IR | μM ATMi |

The microarray data is analyzed using a range of tools. The samples in the groups above are compared to define IR-dependent (parameters 2 and 3, condition A vs B) and EBV-dependent stress gene expression signatures (parameter 1, condition A vs B). An ATM-dependent signature is defined in each of the activated groups (condition B vs C). The differentially expressed genes are analyzed using pathway analysis tools including PANTHER (Mi, H., et al., (2007). Nucleic Acids Res 35, D247-252) and GATHER (Chang, J. T., and Nevins, J. R., (2006) Bioinformatics 22, 2926-2933) towards understanding the underlying molecular basis for these differences and towards providing additional targets for inhibition in a host innate response to activator-mediated proliferative signals. The robustness of the ATM phenotype and specificity of the inhibitor give confidence to this set of experiments towards defining the ATM-dependent gene expression profile that distinguishes oncogenic from exogenous genotoxic stress.

Also incorporated is the analysis of oncogene and tumor suppressor gene expression signatures (Bild, A. H., et al. (2006a). Nat Rev Cancer 6, 735-741; Bild, A. H., et al. (2006b). Nature 439, 353-357). The data is also compared to previously published expression data analyzing IR-induced changes in EBV-transformed cells from normal or A-T individuals (Elkon, R., et al., (2005). Genome Biol 6, R43). IR-induced and ATM-dependent changes in these cells were largely found to be regulated by the p53 and NFκB signaling pathways. These pathways are known to be important for the balance of life and death in EBV transformed cells (Cahir-McFarland, E. D., et al. (2000). Proc Natl Acad Sci USA 97, 6055-6060; Forte, E., and Luftig, M. A. (2009). J Virol 83, 2491-2499). However, lithe is known about their regulation at the genomic level early after infection. This is addressed by analyzing the upstream promoter sequences of the genes regulated by EBV and ATM (parameter 1, condition B vs C) for consensus NFκB or p53 binding sites.

Another approach involves ruling out confounding expression due simply to proliferation. In this approach, LCLs are used as a proliferating control and other forms of proliferation such as CpG are considered to rule out such differences. Also considered are other forms of DNA damage to activate ATM. Moreover, experiments using the Chk1 and Chk2 inhibitors are employed to assess the relative contributions of these kinases in the ATM-dependent response to EBV-induced oncogenic stress.

EBV-specific signaling pathway downstream of oncogenic stress that is distinct from genotoxic DNA damage can be defined. Defining such pathways that specifically restrict immortalization can also lead to transcriptional targets for inhibition in a host innate response to activator-mediated proliferative signals.

A technological hurdle involves reverse genetic experiments in primary B cells. Two different approaches to this problem are employed. First, albeit at low efficiency (˜1%), primary resting B cells are transduced with lentivirus expressing shRNA constructs and GFP. Secondly, at somewhat higher efficiency (5-10%), activated primary B cells are transduced with similar constructs. The response to ATMi in both of these settings is preserved. Therefore, shRNA knock-down of several key targets in this pathway can assess their importance in negatively regulating transformation. This assay provides reverse genetic studies of the effectors that are identified in this Example using microarray expression analysis. Furthermore, the ability to transduce primary B cells provides the opportunity to rescue genes with shRNA-resistant alleles and mutations that confer altered specificity to downstream effectors and binding partners.

### Example 13

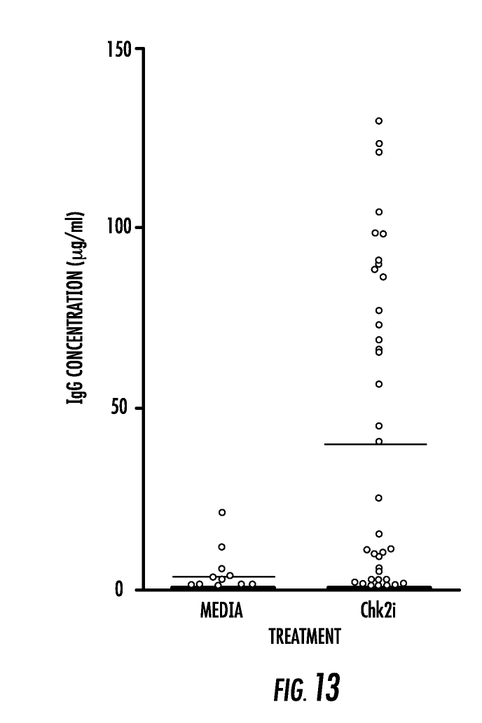

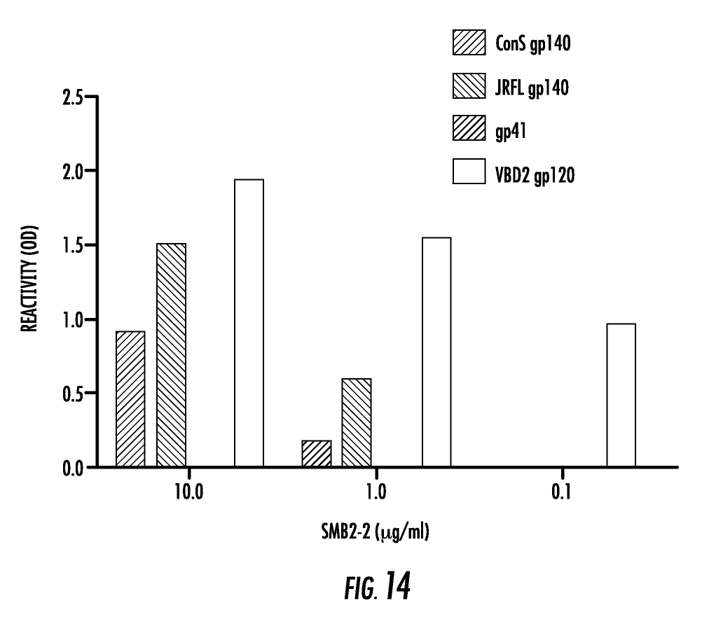

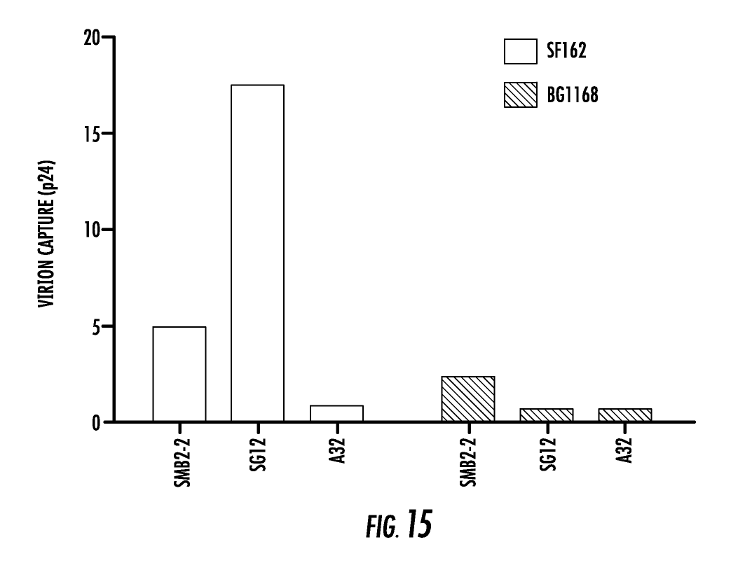

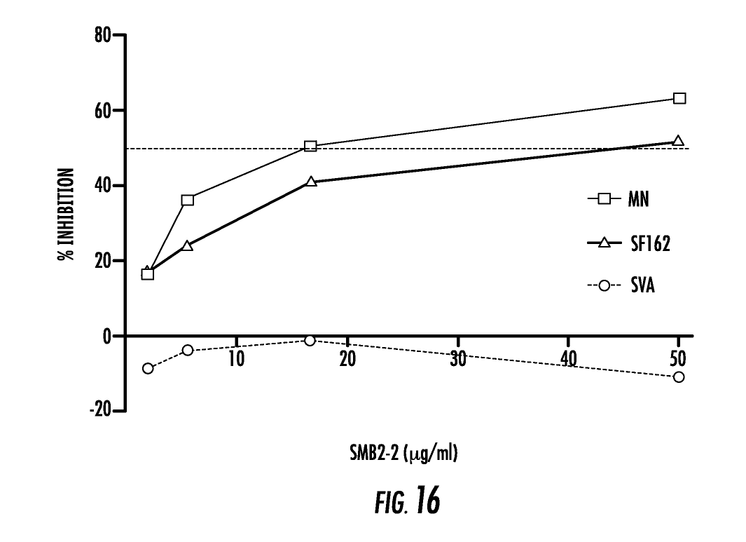

Identification of a HIV-Specific Neutralizing Antibody Following EBV Transformation of Memory B Cells from a Patient Chronically Infected with HIV in the Presence of an Inhibitor of the DNA Damage Response Using the methods of the presently disclosed subject matter, memory B cells from a chronic HIV-1-infected patient were EBV transformed. An increase in the frequency of IgG positive cell clones in the presence of Chk2i II was observed (FIG. 13). It was then determined whether these antibodies would specifically recognize HIV glycoproteins and whether they had biological (e.g. neutralizing) activity. Several potentially HIV-1 neutralizing antibodies were identified during initial screening of the cell culture supernatants. One of these was cloned into a hetero-hybridoma cell line and the IgG mAb was purified (SMB2-2). The mAb bound to HIV-1 gp120 and gp140 proteins (FIG. 14) and captured HIV-1 virions (FIG. 15) and neutralized infection of HIV-1 strains SF162 and MN in vitro (FIG. 16). In preliminary experiments, SMB2-2 neutralized infection of HIV-1 strains SF162 and MN in vitro with an IC₅₀˜10 μg/mL. This finding indicates that inhibition of a host innate response to activator-mediated proliferative signals does not preclude the identification of an antigen-specific biologically active antibody. Furthermore, this provides additional evidence indicating the utility in suppressing such innate responses in any method to identify such antibodies.

### REFERENCES

1. Alfieri et al. (1991) Virology 181:595-608.
2. Altschul et al. (1990) J Mol Biol 215:403-410.
3. Antibodies, A Laboratory Manual, Cold Spring Harbor Laboratory, Ed Harlow and David Lane (1988).
4. Arienti et al., 2005 J. Med Chem 48:1873-1875).
5. Ausubel (1995) Short Protocols in Molecular Biology, 3rd ed. Wiley, New York.
6. Babcock et al. (2000) Proc Natl Acad Sci USA 97:12250-12255.
7. Bakkenist et al. (2003) Nature 421:499-506.
8. Bartek and Lucas (2007) Curr Opin Cell Biol 19:238-245.
9. Bartkova et al. (2005) Nature 434:864-870.
10. Bartkova et al. (2006) Nature 444:633-637.
11. Barton (1998) Acta Crystallogr D Biol Crystallogr 54:1139-1146.
12. Bass (2001) Nature 411:428-429.
13. Batzer et al. (1991) Nucleic Acids Res 19:5081.
14. Bauminger & Wilchek (1980) Meth. Enzymol. 70:151-159.
15. Bernasconi et al. (2003) Blood 101:4500-4504.
16. Bodanszky (1993) Principles of Peptide Synthesis, 2nd rev. ed. Springer-Verlag, Berlin/New York.
17. Burma et al. (2001) J Biol Chem 276:42462-42467.
18. Champe et al. (1995) J. Biol. Chem. 270:1388-1394.
19. Cheng (1996) Hum. Gene Ther. 7:275-282.
20. Chothia and Lesk (1987) J. Mol. Biol. 196:901-917.
21. David et al. (1974) Biochemistry 13:1014.
22. Di Micco et al. (2006) Nature 444:638-642.
23. Diebold et al., “Innate Antiviral Responses by Means of TLR7-Mediated Recognition of Single-Stranded RNA,” Science (2004 Feb. 19) [pub ahead of print].
24. Ding et al. (2006) Trends Pharmacol Sci 27:338-344.
25. DiTullio et al. (2002) Nature Cell Biology 4:998-1002.
26. Drosten et al. (2003) N. Engl. J. Med. 348 (20):1967-76.
27. Elbashir et al. (2001a) Nature 411:494-498.
28. European Patent No. 0 439 095.
29. Fields et al. (1996). “Virology,” Lippincott-Raven, New York. Also“Fields'Virology”, 4th edition (2001) editors in chief Knipe & Howley, ISBN 0-7817-1832-5.
30. Glover & Hames (1995) DNA Cloning: A Practical Approach, 2nd ed. IRL Press at Oxford University Press, Oxford/New York.
31. Goldman et al. (1997) Cancer Res 57:1447-1451.
32. Gorgoulis et al. (2005) Nature 434:907-913.
33. Halazonetis et al. (2008) Science 319:1352-1355.
34. Halazonetis (2004) DNA Repair 3:1057-1062.
35. Hartmann and Krieg (2000) The Journal of Immunology 164: 944-952.
36. Hartmann et al. (2000) J. Immunol. 164 (3):1617-24.
37. Hawkins et al. (2007) Nat Protoc 2:2057-2067.
38. Heil et al., “Species-Specific Recognition of Single-Stranded RNA via Toll-like Receptor 7 and 8,” Science (2004 Feb. 19) [pub ahead of print].
39. Hemmi et al. (2002). Nat. Immunol. 3 (2):196-200.
40. Henderson et al. (1977) Virology 76:152-163.
41. Henikoff & Henikoff (1992) Proc Natl Acad Sci USA 89:10915-10919.
42. Henikoff et al. (2000) Electrophoresis 21:1700-1706.
43. Hickson et al. (2004) Cancer Research 64:9152-9159.
44. Hollick et al., 2003 Bioorg. Med. Chem. Lett. 13:3083-3086
45. Huang et al. (2000) Pac Symp Biocomput 5:227-238.
46. Hunter et al. (1962) Nature 144:945.
47. Jazayeri et al. (2006) Nat Cell Biol 8:37-45.
48. Joseph et al. (2000) J Virol 74:9964-9971.
49. Kabat et al., Sequences of Proteins of Immunological Interest, 5th Ed. Public Health Service, National Institutes of Health, Bethesda, Md. (1991).
50. Kaiser et al. (1999) J Virol 73:4481-4484.
51. Karlin & Altschul (1993) Proc Natl Acad Sci USA 90:5873-5877.
52. Kieff and Rickinson (2006). “Epstein-Barr virus and its replication,” In Fields Virology, D. M. Knipe, and P. M. Howley, eds. (Philadelphia, Lippincott, Williams, and Wilkins), pp. 2603-2654.
53. Kohler & Milstein (1975) Nature 256 (5517):495-497.
54. Kohler and Milstein (1995) Nature 256:495.
55. Kozbor & Roder (1981) J. Immunol. 127 (4):1275-80.
56. Krieg (2002) Annu. Rev. Immunol. 20:709-760.
57. Krieg et al. (1995) Nature 374 (6522):546-9.
58. Kvell et al. (November 2005) Molecular Therapy 12 (5):892-899.
59. Kyte & Doolittle (1982) J Mol Biol 157:105-132.
60. Lanzavecchia (1985) Nature 314(6011):537-539.
61. Lundgren et al. (1983) J Immunol 131 (4):2000-2003.
62. Mage et al., Monoclonal Antibody Production Techniques and Applications, pp. 79-97 (Marcel Dekker, Inc.: New York, 1987).
63. Mallette et al. (2007) Genes Dev 21:43-48.
64. Manome et al. (1994) Cancer Res 54:5408-5413.
65. McCafferty et al. (1990) Nature 348:552-554 (1990),
66. Medzhitov & Janeway (2002) Science 296:298-300.
67. Medzhitov & Janeway (2000) Immunol Rev 173: 89-97.
68. Nabel (1997) Vectors for Gene Therapy. In Current Protocols in Human Genetics, John Wiley & Sons, New York.
69. Needleman & Wunsch (1970) J Mol Biol 48:443-453.
70. Neri et al. (1997) Nat. Biotechnol. 15:1271-1275.
71. Nuciforo et al. (2007) Carcinogenesis 28:2082-2088.
72. Nygren (1982) J. Histochem. and Cytochem. 30:407.
73. Ohtsuka et al. (1985) J. Biol. Chem. 260:2605 2608.
74. Pain et al., (1981) J. Immunol. Meth., 40:219.
75. Park et al. (1997) Adv. Pharmacol. 40:399-435.
76. Pasqualini et al. (1997) Nat. Biotechnol. 15:542-546.
77. Paulsen and Cimprich (2007) DNA Repair (Amst) 6:953-966.
78. PCT Publication No. WO 00/01846
79. PCT Publication No. WO 00/44895
80. PCT Publication No. WO 00/44914
81. PCT Publication No. WO 01/29058
82. PCT Publication No. WO 01/36646
83. PCT Publication No. WO 89/06692
84. PCT Publication No. WO 95/27062
85. PCT Publication No. WO 99/07409
86. PCT Publication No. WO 99/32619
87. PCT Publication No. WO/2004/076677
88. Pearson & Lipman (1988) Proc Natl Acad Sci USA 85:2444-2448.
89. Pusapati et al. (2006) Proc Natl Acad Sci USA 103:1446-1451.
90. Rosen et al. (1983) J Immunol 130 (6):2899-902.
91. Rosen et al. (1977) Nature 267:52-54 (1977).
92. Rossolini et al. (1994) Mol Cell Probes 8:91-98.
93. Roughan and Thorley-Lawson (2009) J Virol 83:3968-3976.
94. Saltzman & Fung (1997) Adv Drug Deliv Rev 26:209-230).
95. Sambrook & Russell (2001) Molecular Cloning: a Laboratory Manual, 3rd ed. Cold Spring Harbor Laboratory Press, Cold Spring Harbor, N.Y.
96. Saqi et al. (1999) Bioinformatics 15:521-522.
97. Schneider & Eberle (1993) Peptides, 1992: Proceedings of the Twenty-Second European Peptide Symposium, Sep. 13-19, 1992, Interlaken, Switzerland, Escom, Leiden.
98. Schneider et al. (1999) J. Exp. Med. 189 (11):1747-56.
99. Schröder & Lübke (1965) The Peptides, Academic Press, New York.
100. Shannon-Lowe et al. (2005) The Journal of General Virology 86:3009-3019.
101. Shawler et al. (1988) J. Biol. Response Mod. 7:608-618).
102. Silhavy et al. (1984) Experiments with Gene Fusions. Cold Spring Harbor Laboratory, Cold Spring Harbor, N.Y.
103. Sinclair et al. (1994) Embo J 13:3321-3328.
104. Smith & Waterman (1981) Adv Appl Math 2:482-489.
105. Solier and Pommier (2009) Cell Cycle 8:1853-1859.
106. Solier et al. (2009) Mol Cell Biol 29:68-82.
107. Souza et al. (2005) Proc Natl Acad Sci USA 102:18093-18098.
108. Steinitz et al. (1980) Nature 287 (5781):443-445.
109. Steinitz et al. (1984) J. Immunol. 132 (2):877-82.
110. Steinitz et al. (1977) Nature 269 (5627):420-422.
111. Stiff et al. (2006) EMBO J. 25:5775-5782.
112. Sugden and Mark (1977) J Virol 23:503-508.
113. Suto et al. (1991) Anticancer Drug Des. 6:107
114. Takeda et al. (2003) Annu Rev Immunol 21, 335-376.
115. Thomson et al. (1986) J Immunol Methods 94 (1-2):7-12.
116. Thorley-Lawson and Gross (2004) N Engl J Med 350:1328-1337.
117. Tijssen (1993) Laboratory Techniques in Biochemistry and Molecular Biology—Hybridization with Nucleic Acid Probes. Elsevier, New York.
118. Tsang et al. (1991) J Virol 65:6765-6771.
119. U.S. Pat. No. 4,376,110.
120. U.S. Pat. No. 4,551,482.
121. U.S. Pat. No. 4,554,101.
122. U.S. Pat. No. 4,816,567.
123. U.S. Pat. No. 4,997,764.
124. U.S. Pat. No. 5,490,840.
125. U.S. Pat. No. 5,500,362.
126. U.S. Pat. No. 5,510,103.
127. U.S. Pat. No. 5,580,717.
128. U.S. Pat. No. 5,651,991.
129. U.S. Pat. No. 5,688,931.
130. U.S. Pat. No. 5,702,892.
131. U.S. Pat. No. 5,714,166.
132. U.S. Pat. No. 5,739,277.
133. U.S. Pat. No. 5,786,387.
134. U.S. Pat. No. 5,855,900.
135. U.S. Pat. No. 5,858,410.
136. U.S. Pat. No. 5,922,356.
137. U.S. Pat. No. 5,922,545.
138. U.S. Pat. No. 5,939,598.
139. U.S. Pat. No. 5,994,392.
140. U.S. Pat. No. 6,071,890.
141. U.S. Pat. No. 6,106,866.
142. U.S. Pat. No. 6,225,447
143. U.S. Pat. No. 6,593,081
144. Wang et al. (1990) J Virol 64:3407-3416.
145. Wang et al. (1991) J Virol 65:4101-4106.
146. Werther et al. (1996) J. Immunol. 157:4986-4995.
147. Yates et al. (1985) Nature 313:812-815.
148. Zimber-Strobl et al. (1991) J Virol 65:415-423.
149. Zimber-Strobl et al. (1993) The EMBO Journal 12:167-175.
150. Zola, Monoclonal Antibodies: A Manual of Techniques, CRC Press, Inc. (1987) pp. 147-158.

All references listed in the instant disclosure, including but not limited to all patents, patent applications, and scientific journals are incorporated herein by reference in their entireties to the extent that they supplement, explain, provide a background for or teach methodology, techniques and/or compositions employed herein.

It will be understood that various details of the presently disclosed subject matter can be changed without departing from the scope of the present disclosure. Furthermore, the foregoing description is for the purpose of illustration only, and not for the purpose of limitation

What is claimed is:

1. A method of inhibiting a host innate response to activator-mediated proliferative signals in a primary B cell, the method comprising administering to a cell an inhibitor of a host innate response to activator-mediated proliferative signals.

2. The method of claim 1, wherein the host innate response to activator-mediated proliferative signals comprises a DNA damage response (DDR) pathway.

3. The method of claim 2, wherein the inhibitor of a host innate response to activator-mediated proliferative signals comprises a small molecule, a nucleic acid molecule capable of mediating RNA interference, or combinations thereof.

4. The method of claim 3, wherein the small molecule, nucleic acid molecule capable of mediating RNA interference, or combinations thereof, inhibits a component of the DDR pathway.

5. The method of claim 4, wherein the component of the DDR pathway comprises Chk1, Chk2, ATM, ATR, DNA-PK, PARP family members, Tip60 or combinations thereof.

6. The method of claim 1, wherein the primary B cell is a human primary B cell.

7. A method for producing immortalized primary B cells, the method comprising transforming primary B cells using Epstein Barr virus (EBV) in the presence of an inhibitor of a host innate response to EBV-mediated proliferative signals.

8. The method of claim 7, wherein the host innate response to activator-mediated proliferative signals comprises a DNA damage response (DDR) pathway.

9. The method of claim 8, wherein the inhibitor of a host innate response to activator-mediated proliferative signals comprises a small molecule, a nucleic acid molecule capable of mediating RNA interference, or combinations thereof.

10. The method of claim 9, wherein the small molecule, nucleic acid molecule capable of mediating RNA interference, or combinations thereof, inhibits a component of the DDR pathway.

11. The method of claim 10, wherein the component of the DDR pathway comprises Chk1, Chk2, ATM, ATR, DNA-PK, PARP or combinations thereof.

12. The method of claim 7, wherein the primary B cell is a human primary B cell.

13. The method of claim 7, wherein the primary B cell is obtained from a subject infected with or possessing an antigen of interest.

14. A method for increasing efficiency of EBV transformation of primary B cells, the method comprising:
(i) providing a primary B cell; and
(ii) transforming the primary B cell using EBV in the presence of an inhibitor of a host innate response to EBV-mediated proliferative signals,
wherein the efficiency of the EBV transformation of the primary B cell is increased compared to EBV transformation without the use of an inhibitor of a host innate response to EBV-mediated proliferative signals.

15. The method of claim 14, wherein a host innate response to activator-mediated proliferative signals comprises the DNA damage response (DDR) pathway.

16. The method of claim 15, wherein the inhibitor of a host innate response to activator-mediated proliferative signals comprises a small molecule, a nucleic acid molecule capable of mediating RNA interference, or combinations thereof.

17. The method of claim 16, wherein the small molecule, a nucleic acid molecule capable of mediating RNA interference, or combinations thereof, inhibits a component of the DDR pathway.

18. The method of claim 17, wherein the component of the DDR pathway comprises Chk1, Chk2, ATM, ATR, DNA-PK, PARP or combinations thereof.

19. The method of claim 14, wherein the primary B cell is a human primary B cell.

20. The method of claim 14, wherein the primary B cell is obtained from a subject infected with or possessing an antigen of interest.

21. A method for increasing proliferation of primary B cells in culture, the method comprising providing to a primary B cell in culture an activator of B cell proliferation in the presence of an inhibitor of a host innate response to activator-mediated proliferative signals.

22. The method of claim 21, wherein the host innate response to activator-mediated proliferative signals comprises a DNA damage response (DDR) pathway.

23. The method of claim 22, wherein the inhibitor of a host innate response to activator-mediated proliferative signals comprises a small molecule, a nucleic acid molecule capable of mediating RNA interference, or combinations thereof.

24. The method of claim 23, wherein the small molecule, nucleic acid molecule capable of mediating RNA interference, or combinations thereof, inhibits a component of the DDR pathway.

25. The method of claim 24, wherein the component of the DDR pathway comprises Chk1, Chk2, ATM, ATR, DNA-PK, PARP or combinations thereof.

26. The method of claim 21, wherein the activator is a polyclonal activator.

27. The method of claim 26, wherein the polyclonal activator comprises an agonist of a Toll Like Receptor (TLR) which is expressed on B cells.

28. The method of claim 27, wherein the TLR is selected from the group consisting of TLR-7, TLR-9, TLR-10 and combinations thereof.

29. The method of claim 26, wherein the polyclonal activator is selected from the group consisting of: CpG oligodeoxynucleotide; R-848; CD40L; BAFF; cells that express CD40L or BAFF; and monoclonal antibodies, small molecules, or nucleic acids that mimic one or more effects of the foregoing activators.

30. The method of claim 21, wherein the activator is a B cell mitogen or combinations of B cell mitogens.

31. The method of claim 30, wherein the B cell mitogen or combination of B cell mitogens is selected from the group consisting of mitogenic cytokine, IL-2, IL-4, IL-5, IL-10, IL-15, CD40L, a CD40 agonist, and anti-Ig.

32. The method of claim 21, wherein the activator is a CpG oligodeoxynucleotide and the inhibitor is a Chk2 inhibitor.

33. The method of claim 21, wherein the activator is EBV.

34. The method of claim 21, wherein EBV transforms the primary B cell.

35. The method of claim 21, wherein the primary B cell is a human primary B cell.

36. A method for producing a clone of an immortalized primary B cell capable of producing a monoclonal antibody with a desired antigen specificity, the method comprising:
(i) transforming a population of cells comprising primary B cells with EBV in the presence of an inhibitor of a host innate response to EBV-mediated proliferative signals;
(ii) screening the supernatant from the population of cells for antigen specificity; and
(iii) isolating from the population of cells an immortalized B cell clone capable of producing a monoclonal antibody having the desired antigen specificity.

37. The method of claim 36, wherein the host innate response to EBV-mediated proliferative signals comprises a DNA damage response (DDR) pathway.

38. The method of claim 37, wherein the inhibitor of a host innate response to EBV-mediated proliferative signals comprises a small molecule, a nucleic acid molecule capable of mediating RNA interference, or combinations thereof.

39. The method of claim 38, wherein the small molecule, nucleic acid molecule capable of mediating RNA interference, or combinations thereof, inhibits a component of the DDR pathway.

40. The method of claim 39, wherein the component of the DDR pathway comprises Chk1, Chk2, ATM, ATR, DNA-PK, PARP or combinations thereof.

41. The method of claim 36, wherein the population of cells comprising primary B cells is derived from a human.

42. The method of claim 36, wherein the desired antigen specificity of the antibody is directed to a pathogen, allergen, tumor antigen, autoantigen, alloantigen, microbial pathogen, chemical or toxin.

43. The method of claim 42, wherein the desired antigen specificity of the antibody is against a pathogen selected from the group consisting of: HIV, P. falciparium, P. vivax, P. malariae, P. ovale, hepatitis A, hepatitis B, hepatitis C, Measles virus, Ebola virus, Poxviruses, Influenza virus, H1N1 virus, Avian influenza virus, herpes simplex virus type 1 or type 2; SARS coronavirus, mumps virus, rubella virus, rabies virus, papillomavirus, vaccinia virus, varicella-zoster virus, variola virus, polio virus, rhinoviruses, respiratory syncytial virus, a human endogenous retroviruses, Dengue virus, Corynebacterium diphtheriae, Clostridium tetani, Clostridium botulinum, Bordetella pertussis, Haemophilus influenzae, Neisseria meningitidis, serogroup A, B, C, W135 and/or Y; Streptococcus pneumoniae, Streptococcus agalactiae, Streptococcus pyogenes, Staplhylococcus aureus, Bacillus anthracis, Moraxella catarrhalis, Chlaymdia trachomatis, Chlamydia pneumoniae, Yersinia pestis, Francisella tularensis, Salmonella species, Vibrio cholerae, toxic E. coli, and other viral or microbial pathogen.

44. A clone of an immortalized primary B cell produced according to the method of claim 36.

45. A method for producing a monoclonal antibody, comprising the steps of:
(i) providing to a primary B cell in culture an activator of B cell proliferation in the presence of an inhibitor of a host innate response to activator-mediated proliferative signals;
(ii) screening the supernatant from the culture for antigen specificity;
(iii) isolating from the culture a B cell capable of producing a monoclonal antibody having the desired antigen specificity; and
(iv) expressing the monoclonal antibody, comprising
(a) culturing the B cell under conditions where the monoclonal antibody is expressed; or
(b) obtaining a nucleic acid encoding the antibody of interest from the isolated B cell;
(c) employing the nucleic acid to prepare an expression host that can express the antibody of interest; and
(d) culturing the expression host under conditions where the antibody of interest is expressed.

46. The method of claim 45, wherein the host innate response to activator-mediated proliferative signals comprises a DNA damage response (DDR) pathway.

47. The method of claim 46, wherein the inhibitor of a host innate response to actvator-mediated proliferative signals comprises a small molecule, a nucleic acid molecule capable of mediating RNA interference, or combinations thereof.

48. The method of claim 47, wherein the small molecule, nucleic acid molecule capable of mediating RNA interference, or combinations thereof, inhibits a component of the DDR pathway.

49. The method of claim 48, wherein the component of the DDR pathway comprises Chk1, Chk2, ATM, ATR, DNA-PK, PARP or combinations thereof.

50. The method of claim 45, wherein the population of cells comprising primary B cells is derived from a human.

51. The method of claim 45, wherein the activator is a polyclonal activator.

52. The method of claim 51, wherein the polyclonal activator comprises an agonist of a Toll Like Receptor (TLR) that is expressed on B cells.

53. The method of claim 52, wherein the TLR is selected from the group consisting of TLR-7, TLR-9, TLR-10 and combinations thereof.

54. The method of claim 51, wherein the polyclonal activator is selected from the group consisting of: CpG oligodeoxynucleotide; R-848; CD40L; BAFF; cells that express CD40L or BAFF; and monoclonal antibodies, small molecules, or nucleic acids that mimic the effects of these activators.

55. The method of claim 45, wherein the activator is a mitogenic cytokine.

56. The method of claim 55, wherein the B cell mitogen or combination of B cell mitogens is selected from the group consisting of mitogenic cytokine, IL-2, IL-4, IL-5, IL-10, IL-15, CD40L, a CD40 agonist, and anti-Ig.

57. The method of claim 45, wherein the activator is EBV.

58. The method of claim 57, wherein EBV transforms the primary B cell.

59. The method of claim 45, wherein the desired antigen specificity of the antibody is directed to a pathogen, allergen, tumor antigen, autoantigen, alloantigen, microbial pathogen, chemical or toxin.

60. The method of claim 59, wherein the desired antigen specificity of the antibody is against a pathogen selected from the group consisting of: HIV, P. falciparium, P. vivax, P. malariae, P. ovale, hepatitis A, hepatitis B, hepatitis C, Measles virus, Ebola virus, Poxviruses, Influenza virus, H1N1 virus, Avian influenza virus, herpes simplex virus type 1 or type 2; SARS coronavirus, mumps virus, rubella virus, rabies virus, papillomavirus, vaccinia virus, varicella-zoster virus, variola virus, polio virus, rhinoviruses, respiratory syncytial virus, a human endogenous retroviruses, Dengue virus, Corynebacterium diphtheriae, Clostridium tetani, Clostridium botulinum, Bordetella pertussis, Haemophilus influenzae, Neisseria meningitidis, serogroup A, B, C, W135 and/or Y; Streptococcus pneumoniae, Streptococcus agalactiae, Streptococcus pyogenes, Staplhylococcus aureus, Bacillus anthracis, Moraxella catarrhalis, Chlaymdia trachomatis, Chlamydia pneumoniae, Yersinia pestis, Francisella tularensis, Salmonella species, Vibrio cholerae, toxic E. coli, and other viral or microbial pathogen.

61. The method of claim 45, further comprising step (v) purifying the monoclonal antibody.

62. The method of claim 45, wherein the expression host is a prokaryotic or eukaryotic cell.

63. A monoclonal antibody produced according to the method of claim 45.

64. A pharmaceutical composition comprising a pharmaceutically acceptable carrier and a monoclonal antibody or functional fragment thereof produced according the method of claim 45.

65. A method of treating a subject, comprising administering to the subject a pharmaceutical composition of claim 64.

66. A method of diagnosing a subject, comprising employing the monoclonal antibody of claim 63.

67. A method for identifying a novel broadly neutralizing antibody having a desired antigen specificity, comprising the steps of:
(i) providing a subject infected with or possessing the antigen for which the antibody specificity is desired;
(ii) culturing primary B cells obtained from the subject;
(iii) providing to the cultured primary B cells an activator of B cell proliferation in the presence of an inhibitor of a host innate response to activator-mediated proliferative signals; and
(iv) screening the supernatant from the culture of proliferating primary B cells for a novel broadly neutralizing antibody having the desired antigen specificity.

68. The method of claim 67, wherein the host innate response to activator-mediated proliferative signals comprises the DNA damage response (DDR) pathway.

69. The method of claim 68, wherein the inhibitor of a host innate response to activator-mediated proliferative signals comprises a small molecule, a nucleic acid molecule capable of mediating RNA interference, or combinations thereof.

70. The method of claim 69, wherein the small molecule, nucleic acid molecule capable of mediating RNA interference, or combinations thereof, inhibits a component of the DDR pathway.

71. The method of claim 70, wherein the component of the DDR pathway comprises Chk1, Chk2, ATM, ATR, DNA-PK, PARP or combinations thereof.

72. The method of claim 67, wherein the population of cells comprising primary B cells is derived from a human.

73. The method of claim 67, wherein the activator is a polyclonal activator.

74. The method of claim 73, wherein the polyclonal activator comprises an agonist of a Toll Like Receptor (TLR) which is expressed on B cells.

75. The method of claim 74, wherein the TLR is selected from the group consisting of TLR-7, TLR-9, TLR-10 and combinations thereof.

76. The method of claim 73, wherein the polyclonal activator is selected from the group consisting of: CpG oligodeoxynucleotide; R-848 and other compounds that stimulate TLRs; CD40L; BAFF; cells that express CD40L or BAFF; and monoclonal antibodies, small molecules, or nucleic acids that mimic the effects of these activators.

77. The method of claim 67, wherein the activator is a mitogenic cytokine.

78. The method of claim 77, wherein the B cell mitogen or combination of B cell mitogens is selected from the group consisting of mitogenic cytokine, IL-2, IL-4, IL-5, IL-10, IL-15, CD40L, a CD40 agonist, and anti-Ig.

79. The method of claim 67, wherein the activator is EBV.

80. The method of claim 79, wherein EBV transforms the primary B cells.

81. The method of claim 67, wherein the desired antigen specificity of the antibody is directed to any human pathogen, allergen, tumor antigen, autoantigen, alloantigen, microbial pathogen, chemical or toxin.

82. The method of claim 81, wherein the desired antigen specificity of the antibody is selected from the group consisting of: HIV, P. falciparium, P. vivax, P. malariae, P. ovale, hepatitis A, hepatitis B, hepatitis C, Measles virus, Ebola virus, Poxviruses, Influenza virus, H1N1 virus, Avian influenza virus, herpes simplex virus type 1 or type 2; SARS coronavirus, mumps virus, rubella virus, rabies virus, papillomavirus, vaccinia virus, varicella-zoster virus, variola virus, polio virus, rhinoviruses, respiratory syncytial virus, a human endogenous retroviruses, Dengue virus, Corynebacterium diphtheriae, Clostridium tetani, Clostridium botulinum, Bordetella pertussis, Haemophilus influenzae, Neisseria meningitidis, serogroup A, B, C, W135 and/or Y; Streptococcus pneumoniae, Streptococcus agalactiae, Streptococcus pyogenes, Staplhylococcus aureus, Bacillus anthracis, Moraxella catarrhalis, Chlaymdia trachomatis, Chlamydia pneumoniae, Yersinia pestis, Francisella tularensis, Salmonella species, Vibrio cholerae, toxic E. coli, and other viral or microbial pathogen.

83. The method of claim 67, further comprising step (v) purifying the novel broadly neutralizing antibody having a desired antigen specificity.

84. A novel broadly neutralizing antibody having a desired antigen specificity produced according to the method of claim 67.

85. A pharmaceutical composition comprising a pharmaceutically acceptable carrier and a novel broadly neutralizing antibody or functional fragment thereof produced according the method of claim 67.

86. A method of treating a subject, comprising administering to the subject a pharmaceutical composition of claim 85.

87. A method of diagnosing a subject, comprising employing the novel broadly neutralizing antibody of claim 84.

88. The method of claim 80, further comprising the steps of:
(v) isolating from the culture a B cell clone capable of producing a novel broadly neutralizing antibody having a desired antigen specificity; and
(vi) culturing the B cell clone under conditions where the novel broadly neutralizing antibody having a desired antigen specificity is expressed.

89. The method of claim 80, further comprising the steps of:
(v) isolating from the culture a B cell clone capable of producing a novel broadly neutralizing antibody having a desired antigen specificity;
(vi) obtaining and/or sequencing a nucleic acid for the novel broadly neutralizing antibody from the selected B cell clone;
(vii) employing the nucleic acid to prepare an expression host that can express the antibody of interest; and
(viii) culturing the expression host under conditions where the novel broadly neutralizing antibody having a desired antigen specificity is expressed.

90. The method of claim 89, wherein the expression host is a prokaryotic or eukaryotic cell.

91. A method for generating a profile of the humoral response of a subject, comprising:
(i) culturing primary B cells obtained from a subject;
(ii) providing to the cultured primary B cells an activator of B cell proliferation in the presence of an inhibitor of a host innate response to activator-mediated proliferative signals;
(iii) screening the supernatant from the culture of proliferating primary B cells for one or more antibodies; and
(iv) characterizing the one or more antibodies, wherein a profile of the humoral response of the subject is generated.

92. The method of claim 91, wherein characterizing the one or more antibodies comprises collecting data regarding the specificity, affinity, stability, isotypte, or gene segment sequence preference of the one or more antibodies.

93. The method of one of claims 3, 9, 16, 23, 38, 47 and 69, wherein the nucleic acid molecule capable of mediating RNA interference is selected from the group consisting of small interfering RNA, short interfering RNA, microRNA, short hairpin RNA, and combinations thereof.


SEQUENCE LISTING (1 sequence)

SEQ ID NO: 1 - DNA, 24 bp, Artificial Sequence
  CpG 2006
tcgtcgttttgtcgttttgtcgtt